SEQUENCE LISTING (94 sequences)

SEQ ID NO: 1 - protein, 398 aa, Beta vulgaris
  REGION 1..398: note = misc_feature - BvADH-alpha Big Buck sugar beet variety
MISLSSFHPSSTTATATAATATTHPPQQCPAFSSPPSHLSLPLRHPRQHLVVRCGGGGSASESVFNRDSAATRVSNDHLDVSKRDVKLKIAIIGFGNFGQFLAKTMAKQGHRVLAYSRSDYSRAAKEIGVEYFTDADDLCEEHPEVILLCTSILSTEKVLRSLPLHRLRRSTLFADVLSVKEFPRSLFLQLLPKDFDILCTHPMFGPDSGKDGWGGLPFVFDKVRVGSDQSRTSRAEAFLDVFRNAGCRMVEMSCVDHDKHAAGSQFITHMMGRVLEKLALENTPINTKGYESLLNLVDNTARDSFELFYGLFLYNKNAMEQLDRMDWAFEMVKKQLSGYLHDLVRKQLMLEGNNDQAEVTFDKPLMLPSPTINPPQIVPSADMAEKKHDLVVVNGTR

SEQ ID NO: 2 - protein, 398 aa, Beta vulgaris
  REGION 1..398: note = misc_feature - BvADH-alpha W357B red beet variety
MISLSSFHPSSTTATATAAAATTHPPQQCPAFSSPPSHLSLPLRHPRQHLVVRCGGGGSASESVFNRDSAATRVSNDHLDVSKRDVKLKIAIIGFGNFGQFLAKTMAKQGHRVLAYSRSDYSRAAKEIGVEYFTDADDLCEEHPEVILLCTSILSTEKVLRSLPLHRLRRSTLFADVLSVKEFPRSLFLQLLPKDFDILCTHPMFGPDSGKDGWGGLPFVFDKVRVGSDQSRTSRAEAFLDVFRNAGCRMVEMSCVDHDKHAAGSQFITHMMGRVLEKLALENTPINTKGYESLLNLVDNTARDSFELFYGLFLYNKNAMEQLDRMDWAFEMVKKQLSGYLHDLVRKQLMLEGNNDQAEVTFDKPLMLPSPTINPPQIVPSADMAEKKHDLVVVNGTR

SEQ ID NO: 3 - protein, 398 aa, Beta vulgaris; identical to SEQ ID NO: 2
  REGION 1..398: note = misc_feature - BvADH-alpha Blankoma white beet variety

SEQ ID NO: 4 - protein, 398 aa, Beta vulgaris; identical to SEQ ID NO: 2
  REGION 1..398: note = misc_feature - BvADH-alpha Touch Stone yellow beet variety

SEQ ID NO: 5 - protein, 407 aa, Beta vulgaris
  REGION 1..407: note = misc_feature - BvADH-alpha Sea beet PI562585 variety
MISLSSFHPSSTTATATAATATATAATATATTHPPQQCPAFSSPPSHLSLPLRHPRQHLVVRCGGGGSASESVFNRDSAATRVSNDHLDVSKRDVKLKIAIIGFGNFGQFLAKTMAKQGHRVLAYSRSDYSRAAKEIGVEYFTDADDLCEEHPEVILLCTSILSTEKVLRSLPLHRLRRSTLFADVLSVKEFPRSLFLQLLPKDFDILCTHPMFGPDSGKDGWGGLPFVFDKVRVGSDQSRTSRAEAFLDVFRNAGCRMVEMSCVDHDKHAAGSQFITHMMGRVLEKLALENTPINTKGYESLLNLVDNTARDSFELFYGLFLYNKNAMEQLDRMDWAFEMVKKQLSGYLHDLVRKQLMLEGNNDQAEVTFDKPLMLPSPTINPPQIVPSADMAEKKHDLVVVNGTR

SEQ ID NO: 6 - protein, 346 aa, Spinacia oleracea
  REGION 1..346: note = misc_feature - SoADH-alpha
CAASDSVFNHDIGVPFVSTRASGEVPEVNSRDIKLKIAIIGFGNFGQFLAKTITKQGHRVLAYSRSDYSRAAKEIGVEYFSDADDLCEEHPEVILLCTSILSTEKVLRSLPLHRLRRSTLFVDVLSVKEFPRSLFLQVLPKDFDILCTHPMFGPDSGKSGWGGLPFVFDKVRVGSDPTRAARTEAFLDIYRNAGCRMVEMTCADHDKHAAGSQFITHMMGRVLEKLALENTPINTKGYESLLNLVDNTARDSFELFYGLFLYNKNAMEQLDRMDWAFEMVKKQLSGYLHDLVRKQLMLETTNEQVGFDQTFMLPSPADNPRQTPPSAAVSENSKPDFVVVNGNNSR

SEQ ID NO: 7 - protein, 396 aa, Spinacia oleracea
  REGION 1..396: note = misc_feature - Spergularia marina ADH-alpha
MMNSISFVNSSSTTTADIIYLNHQFSRHKCFSRLPRDATPRDRRKISLARAINGSPTCSHVEIDQTLVSSSQATTRACSNEQKKLKIAVVGFGNFGQFLAREMVKQGHQVLAYSRSDYSKVAKEIGVQFFRDPDDLCEEHPQVVLLCTSILSTEKVLRSLPVDRLRRSTLIVDVLSVKEFPRTLFLRHLPEDLDILCTHPMFGPDSGKSGWDGLPFVFDKVRVGSDPTRTHRVNTFLDIFKHAGCRMVEMTCMDHDKHAAGSQFITHMMGRVLEKVGLSNTPINTKGYESLLNLVDNTARDSFELFYGLFLYNKNAMEELDRLDWAFDTVKMQLSGYLHDFASKKLMLETGNELAGIVSGKIGDDNHNNKRLMLSPPTNSYKNVTFTDTKVSEKMM

SEQ ID NO: 8 - protein, 375 aa, Rivina humilis
  REGION 1..375: note = misc_feature - RhADH-alpha
CTAFTKTNNNNALGYGYGYGYGYGYDKNKVSSTEQGDEVSGSSSNSKKLKIGIIGFGNFGQFMAKTMVKHGHTVLAYSRSDYSRAAHTIGVRYFSDPDDLCEEHPEVILLCTSILSTERVLRSLPLHRLRRSTLVADVLSVKEFPRSLFLQLLPSDFDILCTHPMFGPDSGKAGWGGLPFVFDKVRVGSQPERLTRVEAFLDIFRDAGCRMVEMSCAEHDRHAAGSQFITHMMGRVLEKLALEDTPINTKGYESLLNLVDNTARDSFELFYGLFLYNKNAMEQLDRMHWAFETVKQQLSGYLHVLVRKQLMLETSSGNDNNNTNNINISSGDNINNKDTNNKLMLPSPGISSAKIVPPVQEKEKHDLVMLNGSKR

SEQ ID NO: 9 - protein, 341 aa, Portulaca oleracea
  REGION 1..341: note = misc_feature - PoADH-alpha
CSSSSSSSASIIINGSGSSTTNSSVFDASSSSDSDVKKRSEVKLKIGIIGFGKFGQFLAKRIVSQGHDVLAYSRSDYSRVASEIGVRFFSDADDLCEEHPQVILLCTSILSTERVLRSLPLHRLRRSTLFADVLSVKEFPRSLFLQLLPSDFDILCTHPMFGPDSGKSGWDSLPFVFDKVRVGSTPTRVTRSEAFLDIFRTAGCRMVEMSCAEHDKHAAGSQFITHMMGRVLEKLDLENTPINTRGYESLRNLVDNTARDSFELFYGLFLYNKNATEQLDRMDWAFEMVKKQLSGYLHHLVRKQLMLESSNTHENHVDNKLLLPENKQKQHDLVVVVNDRS

SEQ ID NO: 10 - protein, 385 aa, Spinacia oleracea
  REGION 1..385: note = misc_feature - Paronychia polygonifolia ADHa
MNSISIVSSTKSTYYKVYQFPSPKICFFHPSKLSIPSCHLKFQNFAVRCNSSNNPKNVSNSKDNKWKPSEINKGIKLKIAVVGFGNFGQFLAKEMVKQGHQVVAYSRTDYTKVAQDMGVRFFSDACEMFIEQPEVILMCTSILSTEKVLRSLPLHRLRPATIFVDVLSVKEFPRSLFLQHLPKDFGILCTHPMFGPNSAKAGWAGLPFVLDRVRVSIDPTQATRTEAFLDIFRNAGCRMVEMTCEDHDKHAAGSQFITHMMGRVLEKVGLRNTPINTKGYESLLNLVENTGRDSFELFYGLFLYNENAMVQLERLDWAFKKVKSQLSACMHDHVRESLMFESHGDQNKIMKKASYKSLLSAYTEKSNKIVKDTKIKKDLVISGQQ

SEQ ID NO: 11 - protein, 239 aa, Spinacia oleracea
  REGION 1..239: note = misc_feature - Herniaria latifolia ADHa
VGFGNFGQFLAKEMVKQGHQVLAYSRSDYSRVAQEIGVQYFSNPDDLCKEHPEVILLCTSILSTEKVLNTLPLDRLRPSTLFSDVLSVKEFPRTLFLQQLPEDFDIICTHPMFGPDSGKHGWAGLPYVYDKVRVGLDPTRIRRAEAFLNIFERAGCRMVEMTCAEHDKHAAGSQFITHMLGRVLEKVGLLNTPINTKGYESLLSLVDNTARDSFELFYGLFLYNKNAMEQLDRLDWAFD

SEQ ID NO: 12 - protein, 351 aa, Corrigiola litoralis
  REGION 1..351: note = misc_feature - Corrigiola litoralis ADHa
MNGSADHFHPNIKVNGEVLNPMVGSSDVAEDVKLKIAIVGFGNFGQFLAKEIVKQGHKVLAYSRSDYSKAAKEIGVQYFSDADDLCEEHPEVILLCTSILSTEKVMRALPIHRLRRSTLFVDVLSVKEFPRSLFLQVLPKDFDILCTHPMFGPDSGKAGWGGLPFVFDKVRVAPDSTRATRAEAFLDIFRRAGCRMVEMTCADHDKHAAGSQFITHMMGRVLEKIGLENTPINTKGYESLLNLVDNTARDSFELFYGLFLYNKNAMEQLDRMDWAFEMIKKRLSGYLHDLVRKQLMLETTGNDQAGLTNGAKNNHDKKLMLPPPAANPSMIVPSAATHEKKHDLVHVNGSR

SEQ ID NO: 13 - protein, 325 aa, Spinacia oleracea
  REGION 1..325: note = misc_feature - Telephium imperati ADHa
MVGPSESGKDVKLEIAVVGFGNFGQFLGREIVKQGHEVLAYSRSDYSKVAKEIGVRYFSDAHDLCEEHPEVILLCTSILSTERVLHSLPLNRLRRSTLFVDVLSVKEFPRNLFLQNLPNDFDILCTHPMFGPDSGKAGWDGLPFVFDKVRVGSDPARTTRADTFLDIFRNAGCRMVEMSCAEHDRHAAGSQFITHMMGRVLEKIGLENTPINTKGYESLLNLVDNTARDSFELFLYYKNAMEQLDRMDWAFEMIKKQLSGYLHELVRKQLMLETNNDQSGIINGKTNCDKRLMLPPPAANPSVIVPDPVPAVKKKHDLVHVNGSR

SEQ ID NO: 14 - protein, 360 aa, Beta vulgaris
  REGION 1..360: note = misc_feature - BvADH-beta Big Buck sugar beet variety
MLSLSSTTTAKPSPSPSPANFPAKLSSLSTITTTLSFSPRRRYFHGVKTLTIRSIDAAQFFDYESKLAAINTTSSSSSSSYSKLKIAIVGFGNYGQFLAKTLVSQGHTVLAYSRSDYSKIAANLGVSYFSDPDDLCEEHPEVIMLCTSILSTEVMLNSLPLQRLKRSTLFVDVLSVKEFPRNLFLQTLPSDFDILCTHPMFGPESGKNGWGSLPFVYDKVRIGKDEGRIKRCESFLDVFRREGCRVEEMTCAEHDKFAAGSQFITHFLGRVLEKLDLEDTPINTKGYESLLNLVDNTSKDSFELFYGLFLYNQNAMEQLERLDWAFELVKKQLFGHLHGLLRKQLFGFSEIDERIGKAKE

SEQ ID NO: 15 - protein, 60 aa, Beta vulgaris
  REGION 1..60: note = misc_feature - BvADH-beta W357B red beet variety
MLSLSSTTTAKPSPSPSPANFPAKLSSLSTITTTLSFSPRRRYFHGVKTLTIRSIDAAQF

SEQ ID NO: 16 - protein, 386 aa, Beta vulgaris
  REGION 1..386: note = misc_feature - BvADH-beta Touch Stone yellow beet variety
MLSLSSTTTAKPSPSPSPANFPAKLSSLSTITTTLSFSPRRRYFHGVKTLTIRSIDAAQFFDYESKLAAINTTSSSSSSSYSKLKIAIVGFGNYGQFLAKTLVSQGHTVLAYSRSDYSKIAANLGVSYFSDPDDLCEEHPEVIMLCTSILSTEVMLNSLPLQRLKRSTLFVDVLSVKEFPRNLFLQTLPSDFDILCTHPMFGPESGKNGWGSLPFVYDKVRIGKDEGRIKRCESFLDVFRREGCRVEEMTCAEHDKFAAGSQFITHFLGRVLEKLDLEDTPINTKGYESLLNLVDNTSKDSFELFYGLFLYNQNAMEQLERLDWAFELVKKQLFGHLHGLLRKQLFGFSEIDERIGKAKEIKFLSDAAEQNGSALSARENANSETN

SEQ ID NO: 17 - protein, 386 aa, Beta vulgaris; identical to SEQ ID NO: 16
  REGION 1..386: note = misc_feature - BvADH-beta Blankoma white beet variety

SEQ ID NO: 18 - protein, 386 aa, Beta vulgaris
  REGION 1..386: note = misc_feature - BvADH-beta Sea beet PI562585 variety
MLSLSSTTTAKPSPSPSPANFPAKLSSLSTITTTISFSPRRRYFHGVKTLTIRSIDAAQFFDYESKLAAINTTSSSTSSSYSKLKIAIVGFGNYGQFLAKTLVSQGHTVLAYSRSDYSKIAANLGVSYFSDPDDLCEEHPEVIMLCTSILSTEVMLNSLPLQRLKRSTLFVDVLSVKEFPRNLFLQTLPSDFDILCTHPMFGPESGKNGWGSLPFVYDKVRIGKDEGRIKRCESFLDVFRREGCRVEEMTCAEHDKFAAGSQFITHFLGRVLEKLDLEDTPINTKGYESLLNLVDNTSKDSFELFYGLFLYNQNAMEQLERLDWAFELVKKQLFGHLHGLLRKQLFGFSEIDERIGKAKEIKFLSDAAEQNGSALSARENANSETN

SEQ ID NO: 19 - protein, 321 aa, Spinacia oleracea
  REGION 1..321: note = misc_feature - SoADH-beta
AATNTSTATSSSQSSYSKLKVAIVGFGNYGQFLAKTMVSQGHTVLAYSRSDYSKIAPNLGVSFFSDPDDLCEEHPEVILLCTSILSTEFMLNSLPLQRLKRSTLFVDVLSVKEFPRNLFLQTLPPDFDILCTHPMFGPESGKNGWGGLPFVYDKVRIGKAERRIRRCENFLDVFRRAGCRVEEMTCAEHDKYAAGSQFITHFLGRVLEKLDLEDTPINTKGYESLLNLVDNTSKDSFELFYGLFLYNQNAMEQLERLDWAFELVKKQLFGHLHGLLRGQLFGCTEIDERLEKAKELKFLSDATTQNGSASAPRENANSEIN

SEQ ID NO: 20 - protein, 321 aa, Nepenthes alata
  REGION 1..321: note = misc_feature - NaADH-beta
AALPNDYETKLSHLPSSFAKLKVGIIGFGNYGQFLAKTLVRQGHTVLAHSRSNYSQNAAKLGVSFFYDPNDLCEEHPEVILLCTSILSTESVLRSLPLQRLKRSTLFVDVLSVKEFPRSLLLQILPPDLDILCTHPMFGPESGKNGWSGLPFVYDKVRIGEHEIRVNRCDNFIEVFRREGCRMVQMSCAEHDRHAAGSQFITHMMGRVLEKLKLEDTPINTKGYESLLNLVENTARDSFELFYGLFLYNKNVMEQLERMDLAFEMVKKQLFGHLHGLLRSQLFDGSEMEVRVEEERKLLSDGSQNGHVFSSFSDSKNVERN

SEQ ID NO: 21 - DNA, 1197 bp, Beta vulgaris
  misc_feature 1..1197: note = Beta vulgaris Big Buck arogenate dehydrogenase alpha, complete CDS
  source 1..1197: mol_type = other DNA
atgatttcactctcttcttttcatccttcctccaccaccgccaccgccaccgccgccaccgccaccacccacccaccacaacaatgtcccgctttttcctctcctccatcgcatctctcgcttcctttacgccaccctcgccaacaccttgtagttcggtgcggtggaggtggttcggcctccgaatcggtatttaaccgtgatagtgctgctactcgtgtttctaatgatcatcttgacgttagtaaaagagatgttaagcttaagattgctattattgggtttggtaactttggccagtttttggctaagacaatggctaagcaaggtcatagagtgttggcttactcacgctcggactactcccgcgctgctaaggagatcggcgtcgagtattttactgacgccgatgacctctgcgaggagcaccctgaggttattcttttgtgcacgtccatcctctcaacggagaaggtcctccgatcactccccctccaccggctccgtcgttcaaccctctttgcggatgttctctcggtcaaggaatttcctcgatcgctcttccttcaactacttcctaaggactttgatatcctatgcacccaccctatgtttggcccagactcgggcaaagacgggtggggtggactaccctttgtgtttgataaagttagagtcggatcagatcagagtcggacgtctcgtgctgaggcattcctagacgtgtttaggaatgccgggtgtaggatggtggaaatgagttgtgttgatcatgacaagcatgcagccgggtctcaatttattacacatatgatgggacgagttttggagaaattggccttggaaaatacaccaattaatacaaaagggtacgaaagtttgttaaatttggtggataatactgcaagggatagttttgagttgttttatgggttgtttttgtacaataaaaatgcaatggagcaattggatagaatggattgggctttcgagatggtaaaaaagcaactttcgggatatttgcatgatcttgttagaaaacaattgatgttggagggtaataatgatcaagctgaggttacttttgacaaaccattaatgcttccttctcctactattaatcctccacaaatagttccttctgctgatatggctgagaagaagcatgatttagtggtggttaatggtactagatag

SEQ ID NO: 22 - DNA, 1197 bp, Beta vulgaris
  misc_feature 1..1197: note = Beta vulgaris W357B arogenate dehydrogenase alpha, complete CDS
  source 1..1197: mol_type = other DNA
atgatttcactctcttcttttcatccttcctccaccaccgccaccgccaccgccgccgccgccaccacccacccacctcaacaatgtcccgctttttcctctcctccgtcgcatctctcgcttcctttacgccaccctcgccaacaccttgtagttcggtgcggtggaggtggttcggcctccgaatcggtatttaaccgtgatagtgctgctactcgtgtttctaatgatcatcttgacgttagtaaaagagatgttaagcttaagattgctattattgggtttggtaactttggccagtttttggctaagacaatggctaagcaaggtcatagagtgttggcttactcacgctcggactactcccgcgctgctaaggagatcggcgtcgagtattttactgacgccgatgacctctgcgaggagcaccctgaggttattctgttgtgcacatccatcctctcaacggagaaggtcctccgatcactccccctccaccggctccgtcgttcaaccctctttgcggatgttctctcggtcaaggaatttcctcgatcgctcttccttcaactacttcctaaggactttgatatcctatgcacccaccctatgtttggcccagactcgggcaaagacgggtggggtggactaccttttgtgttcgataaagttagagtcggatcagatcagagtcggacatctcgtgctgaggcattcctagacgtgtttaggaatgccgggtgtaggatggtggaaatgagttgtgttgatcatgacaagcatgcagccgggtctcaatttattacacatatgatgggacgagttttggagaaattggccttggaaaatacaccaattaatacaaaagggtacgaaagtttgttaaatttggtggataatactgcaagggatagttttgagttgttttacgggttgtttttgtacaataaaaatgcaatggagcaattggatagaatggattgggctttcgagatggtaaaaaagcaactttcgggatatttgcatgatcttgttagaaaacaattgatgttggagggtaataatgatcaagctgaggttacttttgacaaaccattgatgcttccttctcctactattaatcctccacaaatagttccctctgctgatatggctgagaagaagcatgatttagtggtggttaatggtactagatag

SEQ ID NO: 23 - DNA, 1197 bp, Beta vulgaris
  misc_feature 1..1197: note = Beta vulgaris Blankoma arogenate dehydrogenase alpha, complete CDS
  source 1..1197: mol_type = other DNA
atgatttcactctcttcttttcatccttcctccaccaccgccaccgccaccgccgccgccgccaccacccacccaccacaacaatgtcccgctttttcctctcctccgtcgcatctctcgcttcctttacgccaccctcgccaacaccttgtagttcggtgcggtggaggtggttcggcctccgaatcggtatttaaccgtgatagtgctgctactcgtgtttctaatgatcatcttgacgttagtaaaagagatgttaagcttaagattgctattattgggtttggtaactttggccagtttttggctaagacaatggctaagcaaggtcatagagtgttggcttactcacgctcggactactcccgcgctgctaaggagatcggcgtcgagtattttactgacgccgatgacctctgcgaggagcaccctgaggttattctgttgtgcacgtccatcctctcaacggagaaggtcctccgatcactccccctccaccggctccgtcgttcaaccctctttgcggatgttctctcggtcaaggaatttcctcgatcgctcttccttcaactacttcctaaggactttgatatcctatgcacccaccctatgtttggcccagactcgggcaaagacgggtggggtggactaccttttgtgttcgataaagttagagtcggatcagatcagagtcggacatctcgtgctgaggcattcctagacgtgtttaggaatgccgggtgtaggatggtggaaatgagttgtgttgatcatgacaagcatgcagccgggtctcaatttattacacatatgatgggacgagttttggagaaattggccttggaaaatacaccaattaatacaaaagggtacgaaagtttgttaaatttggtggataatactgcaagggatagttttgagttgttttacgggttgtttttgtacaataaaaatgcaatggagcaattggatagaatggattgggctttcgagatggtaaaaaagcaactttcgggatatttgcatgatcttgttagaaaacaattgatgttggagggtaataatgatcaagctgaggttacttttgacaaaccattgatgcttccttctcctactattaatcctccacaaatagttccctctgctgatatggctgagaagaagcatgatttagtggtggttaatggtactagatag

SEQ ID NO: 24 - DNA, 1197 bp, Beta vulgaris; identical to SEQ ID NO: 22
  misc_feature 1..1197: note = Beta vulgaris Touch Stone arogenate dehydrogenase alpha, complete CDS
  source 1..1197: mol_type = other DNA

SEQ ID NO: 25 - DNA, 1224 bp, Beta vulgaris
  misc_feature 1..1224: note = Beta vulgaris subsp.maritima PI562585 arogenate dehydrogenase alpha, complete CDS
  source 1..1224: mol_type = other DNA
atgatttcactctcttcttttcatccttcctccaccaccgccaccgccaccgccgccaccgccaccgccaccgccgccaccgccaccgccaccacccacccaccacaacaatgtcccgctttttcctctcctccatcgcatctctcgcttcctttacgccaccctcgccaacaccttgtagttcggtgcggtggaggtggttcggcctccgaatcggtatttaaccgtgatagtgctgctactcgtgtttctaatgatcatcttgacgttagtaaaagagatgttaagcttaagattgctattattgggtttggtaactttggccagtttttggctaagacaatggctaagcaaggtcatagagtgttggcttactcacgctcggactactcccgcgctgctaaggagatcggcgtcgagtattttactgacgccgatgacctctgcgaggagcaccctgaggttattcttttgtgcacgtccatcctctcaacggagaaggtcctccgatcactccccctccaccggctccgtcgttcaaccctctttgcggatgttctctcggtcaaggaatttcctcgatcgctcttccttcaactacttcctaaggactttgatatcctatgcacccaccctatgtttggcccagactcgggcaaagacgggtggggtggactaccctttgtgtttgataaagttagagtcggatcagatcagagtcggacgtctcgtgctgaggcattcctagacgtgtttaggaatgccgggtgtaggatggtggaaatgagttgtgttgatcatgacaagcatgcagccgggtctcaatttattacacatatgatgggacgagttttggagaaattggccttggaaaatacaccaattaatacaaaagggtacgaaagtttgttaaatttggtggataatactgcaagggatagttttgagttgttttatgggttgtttttgtacaataaaaatgcaatggagcaattggatagaatggattgggctttcgagatggtaaaaaagcaactttcgggatatttgcatgatcttgttagaaaacaattgatgttggagggtaataatgatcaagctgaggttacttttgacaaaccattaatgcttccttctcctactattaatcctccacaaatagttccttctgctgatatggctgagaagaagcatgatttagtggtggttaatggtactagatag

SEQ ID NO: 26 - DNA, 1041 bp, Spinacia oleracea
  misc_feature 1..1041: note = Spinacia oleracea arogenate dehydrogenase alpha, partial CDS
  source 1..1041: mol_type = other DNA
tgcgccgcctctgactccgtgttcaaccacgatattggtgtgccttttgtctcaacacgcgcttccggcgaggtgccggaggttaacagtagagatattaagcttaagatcgcgatcattgggttcgggaactttggtcagtttttggctaagactattactaagcaaggtcacagagttttggcttactcccggtcagattactcccgtgctgctaaggagatcggcgtcgagtatttctccgacgccgatgatctttgcgaagagcatcccgaggtgatactcctatgcacttcaatcctctcaacagagaaggtcctccgttcgctccccctccaccgccttcgccggtccaccctcttcgtggacgtcctctcggtgaaggagttcccgcgttcacttttcctccaagtccttcctaaagactttgacatcctttgcacccaccccatgttcggcccagactcaggcaaaagcggatggggtgggctcccctttgtcttcgacaaagtccgagtcgggtcggacccaacccgggcggctcggactgaggcgttcctagacatttataggaacgccgggtgtaggatggtggaaatgacatgcgcggaccacgacaagcacgcggctgggtcgcaattcataacccacatgatgggccgggttttggagaaattagccctcgaaaacacaccgattaacacgaaagggtacgagagtttgttgaacttggtggataatacggcccgggacagctttgagttgttttacggactgtttttgtacaacaagaacgcgatggaacaattggatagaatggattgggctttcgagatggtaaagaagcaactttcgggttatttgcatgatcttgttaggaaacaattgatgctagagactaccaatgaacaagttgggtttgatcagacgttcatgcttccttctcctgccgataatcctcgtcaaacaccaccctcggctgccgtttccgagaattcgaaacccgattttgtggtggtaaatggtaataattctagatag

SEQ ID NO: 27 - DNA, 1128 bp, Rivina humilis
  misc_feature 1..1128: note = Rivina humilis arogenate dehydrogenase alpha, partial CDS
  source 1..1128: mol_type = other DNA
tgcacggccttcactaaaactaataataataatgccttgggttatggttacggttatggttatggttatggctatgacaaaaacaaggtgtctagtactgaacagggtgatgaggtttcgggttcgagttcgaattcgaagaagctgaagattggtataattgggttcgggaactttggtcagtttatggcaaagacgatggtgaaacatggtcacactgtgcttgcttattctcgttccgattactcacgtgctgctcataccatcggtgttcgctacttctctgatcctgatgacttgtgcgaagagcaccctgaggtgattctactgtgcacctccatcttatccactgaaagggtgcttcggtcactaccgcttcatcgcctacgccgctcaacactcgttgcggatgtgctgtcggtcaaggaattcccacgttcactcttcctacaactcctcccttctgactttgacatcctttgcactcatcctatgttcggaccggactccggcaaggccgggtggggcggtcttcctttcgtctttgacaaagtccgggttggatcccaacccgaacgcctcacccgtgttgaggccttcctggacattttccgggatgccgggtgccggatggtggagatgagttgtgctgagcatgacaggcatgctgctgggtcacaattcataacacacatgatgggacgtgtgttagagaagcttgcacttgaggacacaccaattaacaccaaagggtatgagagtttgttgaacttggttgataacactgctagggacagttttgagctgttttatggactctttttatacaacaagaatgcaatggaacagcttgatagaatgcattgggcatttgagacagtgaagcaacagctctctggttatttgcatgtccttgttaggaagcagttgatgttggagacttcttccggtaatgacaataataatactaataatattaatattagcagtggtgataatattaataataaggacacaaataataaattaatgttaccttctcctgggattagttctgctaaaattgttccaccagtacaggagaaggagaaacatgacttggtgatgctcaatggatcaaagcggtag

SEQ ID NO: 28 - DNA, 1026 bp, Portulaca oleracea
  misc_feature 1..1026: note = Portulaca oleracea arogenate dehydrogenase alpha, partial CDS
  source 1..1026: mol_type = other DNA
tgctcatcatcatcatcatccagtgccagcatcatcatcaatggttccggtagctccacgacaaactcgagcgtcttcgatgctagttcttcctccgattcagacgtaaaaaaaaggtcagaagtgaagctgaaaatcgggatcattggatttgggaagtttggacagtttctagcgaagagaattgtgagtcagggtcatgatgtcttggcgtattctcggtcggattactcacgggtggcatcggagattggcgtacggttcttctctgacgccgatgacctctgcgaggagcaccctcaggtgatcctgttatgcacatcaatcctgtcaaccgagcgcgttctgcgctcgcttccactacacaggctccgtcgatccaccctgttcgcggatgtcctgtccgtaaaagagttcccgcggtcactcttcttacaattactcccctccgacttcgacattctatgcacacaccccatgttcggacccgactcaggcaagtccgggtgggacagtcttccctttgtcttcgacaaggtccgggtcggatccacccctactcgggtcacccggtccgaggccttcctagacatcttccggaccgccgggtgtaggatggtggaaatgagctgcgccgagcacgacaaacacgcagccgggtcccagttcataacccatatgatgggccgggttctcgagaagttagacttggaaaacacacccataaacaccagaggatatgagagtttgagaaacctggtggacaacacggcaagggacagctttgagctgttttatggattgtttttgtacaacaaaaacgcgacggagcagcttgacaggatggattgggcattcgagatggttaagaaacaactttctgggtatcttcatcatctagttaggaaacagttgatgttagagagtagtaatacacatgaaaatcatgttgacaacaaattgttgcttccagagaataagcagaagcaacatgacttggtcgtcgtcgtcaacgatagatcatag

SEQ ID NO: 29 - DNA, 1191 bp, Spinacia oleracea
  misc_feature 1..1191: note = Spergularia marina arogenate dehydrogenase alpha mRNA, complete CDS
  source 1..1191: mol_type = other DNA
atgatgaattctatctcctttgtcaactcttcctcaacaacaaccgcggatattatctacttaaaccaccaattttcgcgtcacaagtgtttttctcgtcttcctcgggacgcaactcctagggaccgtcggaagatttccttggctagagccatcaacggctcacctacgtgtagccatgttgaaatcgaccaaacgttggttagctctagccaagctactactagagcttgtagtaatgagcaaaagaagcttaaaatcgcggtcgtagggttcgggaattttggacagtttttggctagagaaatggttaagcaaggacatcaagtgttggcttactctcgctctgattactcaaaggttgctaaagagattggtgtccaattctttagggaccctgatgacctttgcgaggaacatcctcaggtggttcttttatgcacctctattctctcaacggagaaggtccttcgctccctcccggttgaccgccttcgccgttccaccctcattgttgacgtcctctcggttaaggagtttccgcgcacccttttcctccggcacttgcctgaggacttggacatcctttgcacccatccaatgtttggcccggactctggcaagtccgggtgggatgggctaccctttgtatttgataaagtccgagttggatcagacccaacccggacccacagagtcaacacattcttggatatatttaaacacgcagggtgtagaatggttgagatgacgtgtatggaccatgacaagcatgcagccggttcccagtttataacccacatgatgggtcgggtcttagagaaagtgggcctttcaaatacacccattaatacaaaagggtatgagagtttgttgaatttggtggataatacagcaagagatagctttgagttgttttatggactgtttttgtacaacaaaaatgcaatggaggagttggatagattggactgggcctttgatacggtaaaaatgcagctttctgggtatttgcatgattttgctagtaaaaagttgatgttggagactggtaatgaactagctgggattgttagtggtaaaattggcgacgacaatcataataacaagaggttaatgctctcccctcctacaaattcttacaagaatgttacttttactgatacgaaagtttcggagaaaatgatgtga

SEQ ID NO: 30 - DNA, 1158 bp, Spinacia oleracea
  misc_feature 1..1158: note = Paronychia polygonifolia dehydrogenase alpha mRNA, complete CDS
  source 1..1158: mol_type = other DNA
atgaattctatctctattgtaagctctactaagtctacttattacaaagtctaccaatttccatcacctaagatatgtttcttccacccttctaagctctctattccttcttgccaccttaagtttcaaaattttgccgtacgttgcaatagtagtaacaacccaaaaaatgtttcaaactctaaggataataaatggaagcctagtgaaattaacaagggaattaagcttaaaatcgcggtagtggggttcggcaactttgggcagttcttggctaaggaaatggttaagcaaggccatcaagtggtggcgtactctcgtactgattatactaaggttgctcaagatatgggtgttcgcttcttttctgatgcttgtgaaatgttcattgagcaacccgaggtgattctaatgtgcacctctatcctctctacggagaaggtgttgcgctccctccctctccaccgtctccggccagccaccatcttcgtggacgtcctctccgtgaaggagttcccccggtccctcttcctccaacacctccccaaggacttcggcatcctttgcactcacccaatgtttgggccaaactcagccaaggccgggtgggccgggctccccttcgttctagacagggttcgggtcagtattgacccgacccaagccacccggacagaggcattcctagacatattccgaaatgcagggtgtaggatggtggaaatgacttgtgaagaccatgacaagcatgcagccgggtcacagttcataacccacatgatgggtcgggttcttgagaaagtggggctccgaaatacacccattaatacaaaagggtacgaaagtttgttgaatttggtggagaatacaggaagagatagctttgagttgttttatgggttgttcttgtacaatgaaaatgcaatggtgcaattagagaggttggactgggcttttaagaaggttaagagtcaactttctgcatgtatgcatgatcatgttagggagagccttatgtttgagtctcatggagatcaaaataagattatgaaaaaggcgagttacaagtcactcctatcagcctatacagaaaaaagtaataagattgtcaaagatacaaagattaagaaggacttggtgattagtgggcaacaataa

SEQ ID NO: 31 - DNA, 720 bp, Spinacia oleracea
  misc_feature 1..720: note = Herniaria latifolia arogenate dehydrogenase alpha mRNA, partial CDS
  source 1..720: mol_type = other DNA
agtcgggtttgggaactttgggcagttcttagccaaagaaatggtgaagcaaggtcatcaagtgttggcttattctcgctctgattattcaagggttgctcaggagattggcgtacagtatttctcaaatcccgacgacctttgcaaagagcatcctgaggttatcctcctgtgcacatccatcctctccactgaaaaagtcctaaatacccttcccctcgaccgcctccgaccatcaactctcttctccgatgtgctctccgtcaaggaattccctcgtacacttttcctccagcaactacccgaggactttgacatcatctgtacccatccaatgttcggcccggactcgggcaaacacgggtgggcagggctcccctacgtctacgacaaagtacgtgtcgggttggatccgacccggatccgccgagcggaggcatttcttaacattttcgaaagggcagggtgtaggatggtggagatgacgtgtgcagagcatgacaagcatgcagctgggtcccagttcataacccacatgttgggccgagttttggagaaagtgggccttttaaatacgcccattaacacaaaagggtacgagagtttgttgagcttggtggataatacagcaagagacagctttgagttgttttatgggctttttttgtacaacaaaaatgcaatggagcagttggatcgattggattgggcctttgacat

SEQ ID NO: 32 - DNA, 1077 bp, Corrigiola litoralis
  misc_feature 1..1077: note = Corrigiola litoralis arogenate dehydrogenase alpha, partial CDS
  source 1..1077: mol_type = other DNA
tgcagcaagggtgtgcatggcatgaatggctcagctgatcattttcatcctaacattaaggttaatggtgaggttttgaaccctatggttggctctagtgatgtagccgaggatgttaagctaaaaatcgccatagttgggtttggaaacttcggacaattcttggctaaggaaattgttaagcagggtcataaggtgttggcttactctcggtctgattactctaaggctgctaaggagattggtgtgcagtatttttccgatgctgatgacctgtgtgaggagcatcctgaggtgatcctcctttgcacctctatcctctcaacggagaaggtgatgcgcgccctccctatccaccgccttcgccggtccaccctcttcgtcgatgttctctcagtgaaggagttcccccgctcactcttcctccaagttctccctaaggactttgacatcctctgcacccacccaatgttcggccctgactccggcaaagccgggtggggtggactcccttttgtctttgacaaagttcgggttgcgccagactccacccgggctactagggccgaggcatttctagacatcttcagaagagcagggtgccgaatggtagaaatgacttgtgcagaccacgacaagcatgcagcaggatcgcagttcatcacacacatgatgggtcgggtgctagagaaaatagggcttgaaaatactcccatcaacacaaaagggtacgagagtttgctcaatttggtggacaatacggcgagagacagctttgagttgttttatgggttgtttttgtataataagaacgcaatggagcagttagatagaatggactgggcttttgagatgataaagaagcgactttcaggatacttgcatgatcttgttaggaagcagttgatgctagaaactactggtaatgatcaagctggtctaactaacggtgcaaaaaataatcatgacaagaagctcatgcttcctcctcctgctgccaatccttctatgattgttccttctgctgctactcatgagaagaagcatgatttggtgcatgtcaatggaagcagatga

SEQ ID NO: 33 - DNA, 990 bp, Spinacia oleracea
  misc_feature 1..990: note = Telephium imperati arogenate dehydrogenase alpha, partial CDS
  source 1..990: mol_type = other DNA
gtattggggagtatggttggtcctagtgagagtgggaaggatgttaagcttgaaatcgcggtagtcgggttcgggaactttgggcagtttttgggtagggaaattgttaagcaggggcatgaggtgttggcttattctcggtctgattactccaaagttgctaaggagattggtgtacgttatttttccgacgctcatgacttgtgtgaggagcatcctgaggtgatcctcctatgcacatccatcctctcaacagagagggtcctccactccctccctctaaaccgcctccgccgctccaccctcttcgtcgacgtcctctccgtgaaggagttcccccgaaacctcttcctccaaaacctccccaacgacttcgacatcctctgcacccacccaatgttcggcccggactccggcaaagccggctgggacgggctccccttcgtgttcgacaaggtccgggtcgggtcagacccggcccggaccacccgggccgacacattcctagacatattcaggaatgcagggtgcaggatggtggaaatgtcctgtgcagagcatgacaggcacgcagccgggtcacaattcataacccacatgatgggtcgggttttggagaaaatcgggctcgaaaacacacccattaacacaaaagggtacgagagtttgttgaatttggtggataatacagcaagggatagctttgaattgtttttgtattataagaatgcaatggagcaattagataggatggattgggcttttgagatgattaagaagcagctttctgggtatttgcatgagcttgttaggaagcaattgatgctagagactaataatgatcaatccgggataattaatggtaaaactaattgtgataaacgactaatgcttcctcctccggccgctaatccgtctgtaattgttcctgatcctgttcctgctgtgaagaagaagcatgatttggtgcatgtcaatggaagtagatga

SEQ ID NO: 34 - DNA, 1161 bp, Beta vulgaris
  misc_feature 1..1161: note = Beta vulgaris W357B arogenate dehydrogenase betta mRNA, complete CDS
  source 1..1161: mol_type = other DNA
atgctttctctctcctccacaaccaccgcaaaaccctcgccgtcgccatctccggcgaattttccggcgaaactttcttctctctccaccatcaccaccactctctctttctctcctcgccggagatattttcatggcgtcaaaaccctaacaattcgcagcatcgacgccgcacaattcttcgattacgaatcaaaacttgccgccattaacacaacctcttcgtcttcatcttcatcttattcgaagctcaaaatcgcaatcgtagggttcggaaattacggacaatttctcgcgaaaaccctagtttctcaaggtcatactgttctcgcttattctcgctctgattactctaaaatcgctgcgaatctcggcgtttcttacttttctgatcctgatgatctttgcgaagaacatcctgaggtaattatgttgtgtacttcgattttatcaactgaagttatgttgaattcgttaccattgcagcgacttaaacgatcgacgctttttgttgatgttttatcggtgaaagaatttccgcgtaatttgtttcttcaaactttaccgtctgattttgatatattatgtactcatcctatgtttgggcctgaatctgggaaaaatggttggggaagtttgccttttgtttatgataaggttaggattgggaaagatgagggtagaattaagagatgtgagagttttttggatgtttttaggagagaaggttgtagggttgaggaaatgacttgtgctgagcatgataagtttgcagcagggtctcagtttataacacatttcttagggagggttttggagaagcttgatttggaggatacgccgattaatacgaaagggtatgagagtttgttgaatttggtggataatacgtcgaaggatagtttcgagttgttttatgggttgtttttgtataatcagaatgctatggagcagttagagaggttagattgggcgtttgagttggttaagaagcaattgtttggacacttgcatgggttgctaaggaaacagttgtttgggttttctgagatagatgaacgtattgggaaggcgaaggagatcaaatttctctctgatgctgcagaacagaatggctctgccttgtctgctagggagaatgcaaattcggagacaaattga

SEQ ID NO: 35 - DNA, 1161 bp, Beta vulgaris
  misc_feature 1..1161: note = Beta vulgaris Big Buck arogenate dehydrogenase betta mRNA, complete CDS
  source 1..1161: mol_type = other DNA
atgctttctctctcctccacaaccaccgcaaaaccctcgccgtcgccatctccggcgaattttccggcgaaactttcttctctctccaccatcaccaccactctctctttctctcctcgccggagatattttcatggcgtcaaaaccctaacaattcgcagcatcgacgccgcacaattcttcgattacgaatcaaaacttgccgccattaacacaacctcttcgtcttcatcttcatcttattcgaagctcaaaatcgcaatcgtagggttcggaaattacggacaatttctcgcgaaaaccctagtttctcaaggtcatactgttctcgcttattctcgctctgattactctaaaatcgctgcgaatctcggcgtttcttacttttctgatcctgatgatctttgcgaagaacatcctgaggtaattatgttgtgtacttcgattttatcaactgaagttatgttgaattcgttaccattgcagcgacttaaacgatcgacgctttttgttgatgttttatcggtgaaagaatttccgcgtaatttgtttcttcaaactttaccgtctgattttgatatattatgtactcatcctatgtttgggcctgaatctgggaaaaatggttggggaagtttgccttttgtttatgataaggttaggattgggaaagatgagggtagaattaagagatgtgagagttttttggatgtttttaggagagaaggttgtagggttgaggaaatgacttgtgctgagcatgataagtttgcagcagggtctcagtttattacacatttcttagggagggttttggagaagcttgatttggaggatacgccgattaatacgaaagggtatgagagtttgttgaatttggtggataatacgtcgaaggatagtttcgagttgttttatgggttgtttttgtataatcagaatgctatggagcagttagagaggttagattgggcgtttgagttggttaagaagcaattgtttggacacttgcatgggttgctaaggaaacagttgtttgggttttctgagatagatgaacgtattgggaaggcgaaggagatcaaatttctctctgatgctgcagaacagaatggctctgccttgtctgctagggagaatgcaaattcggagacaaattga

SEQ ID NO: 36 - DNA, 1161 bp, Beta vulgaris
  misc_feature 1..1161: note = Beta vulgaris Touch Stone arogenate dehydrogenase betta mRNA, complete CDS
  source 1..1161: mol_type = other DNA
atgctttctctctcctccacaaccaccgcaaaaccctcgccgtcgccatctccggcgaattttccggcgaaactttcttctctctccaccatcaccaccactctctctttctctcctcgccggagatattttcatggcgtcaaaaccctaacaattcgcagcatcgacgccgcacaattcttcgattacgaatcaaaacttgccgccattaacacaacctcttcgtcttcatcttcatcttattcgaagctcaaaatcgcaatcgtagggttcggaaattacggacaatttctcgcgaaaaccctagtttctcaaggtcatactgttctcgcttattctcgctctgattactctaaaatcgctgcgaatctcggcgtttcttacttttctgatcctgatgatctttgcgaagaacatccagaggtaattatgttgtgtacttcgattttatcaactgaagttatgttgaattcgttaccattgcagcgacttaaacgatcgacgctttttgttgatgttttatcggtgaaagaatttccgcgtaatttgtttcttcaaactttaccgtctgattttgatatattatgtactcatcctatgtttgggcctgaatctgggaaaaatggttggggaagtttgccttttgtttatgataaggttaggattgggaaagatgagggtagaattaagagatgtgagagttttttggatgtttttaggagagaaggttgtagggttgaggaaatgacttgtgctgagcatgataagtttgcagcagggtctcagtttataacacatttcttagggagggttttggagaagcttgatttggaggatacgccgattaatacgaaagggtatgagagtttgttgaatttggtggataatacgtcgaaggatagtttcgagttgttttatgggttgtttttgtataatcagaatgctatggagcagttagagaggttagattgggcgtttgagttggttaagaagcaattgtttggacacttgcatgggttgctaaggaaacagttgtttgggttttctgagatagatgaacgtattgggaaggcgaaggagatcaaatttctctctgatgctgcagaacagaatggctctgccttgtctgctagggagaatgcaaattcggagacaaattga

SEQ ID NO: 37 - DNA, 1161 bp, Beta vulgaris; identical to SEQ ID NO: 34
  misc_feature 1..1161: note = Beta vulgaris Blankoma arogenate dehydrogenase betta mRNA, complete CDS
  source 1..1161: mol_type = other DNA

SEQ ID NO: 38 - DNA, 1161 bp, Beta vulgaris
  misc_feature 1..1161: note = Beta vulgaris subsp.maritima PI562585 arogenate dehydrogenase betta, complete CDS
  source 1..1161: mol_type = other DNA
atgctttctctctcctccacaaccaccgcaaaaccctcgccgtcgccatctccggcgaattttccggcaaaactttcttctctctccaccatcaccaccactatctccttctctcctcgccggagatattttcatggcgtcaaaaccctaacaattcgcagcatcgacgctgcacaattcttcgattacgaatcaaaactcgccgccattaacacaacctcttcatctacatcgtcatcttattcgaaactcaaaatcgcaatcgtaggtttcggaaattacggacaatttctcgcgaaaaccctagtttctcaaggtcatactgttctcgcttattctcgctctgattactctaaaatcgctgcgaatctcggcgtttcttacttttctgatcctgatgatctttgcgaagaacatcctgaggtaattatgttgtgtacttcgattttatcaactgaagttatgttgaattcgttaccattgcagcgacttaaacgatcgacgctttttgttgatgttttatcggtgaaagaatttccgcgtaatttgtttcttcaaactttaccgtctgattttgatatattatgtactcatcctatgtttgggcctgaatctgggaaaaatggttggggaagtttgccttttgtttatgataaggttaggattgggaaagatgagggtagaattaagagatgtgagagttttttggatgtttttaggagagaaggttgtagggttgaggaaatgacttgtgctgagcatgataagtttgcagcagggtctcagtttattacacatttcttagggagggttttggagaagcttgatttggaggatacgccgattaatacgaaagggtatgagagtttgttgaatttggtggataatacgtcgaaggatagtttcgagttgttttatgggttgtttttgtataatcagaatgctatggagcagttagagaggttagattgggcatttgagttggttaagaagcaattgtttggacacttgcatgggttgctaaggaaacagttgtttgggttttctgagatagatgaacgtattgggaaggcgaaggagatcaaatttctctctgatgctgcagaacagaatggctctgccttgtctgctagggagaatgcaaattcggagacaaattga

SEQ ID NO: 39 - DNA, 966 bp, Spinacia oleracea
  misc_feature 1..966: note = Spinacia oleracea arogenate dehydrogenase betta, partial CDS
  source 1..966: mol_type = other DNA
gccgctaccaatacctccaccgccacctcttcctcacagtcgtcgtactcgaagctcaaggtggcaatcgttgggttcggaaactatggacaatttctcgcaaaaactatggtttctcaaggtcatactgttcttgcatattctcggtctgattactcgaaaatagctccaaatctgggcgtttcgttcttttccgatcctgatgatttatgtgaagaacatccggaggtaattttgctgtgcacttcgattttatcaactgaatttatgttgaattcactaccattgcaacgtcttaagaggtcgacgctttttgttgatgttttatcggttaaggagtttccccgtaacttgtttcttcagactttgccgcctgattttgatattttatgcactcatcctatgtttggtcctgaatctgggaaaaatggatggggaggtttgccgtttgtttatgataaggttaggattgggaaagcagagcgtagaattaggaggtgtgagaattttttggatgtttttaggagagcagggtgtagggttgaggagatgacttgtgcagagcatgataaatacgcggcgggttcacagtttattacgcatttcctggggagggttttggagaagcttgatttggaggatacaccgattaacacgaaagggtacgagagtttgttgaatttggtggataatacgtcgaaggatagtttcgagttgttttatgggttgtttttgtacaaccagaatgctatggagcagttggagaggttagattgggcattcgagttggttaagaagcagttgtttgggcatttgcatggtttgttaaggggtcagttgtttgggtgtactgagattgatgaacgtcttgagaaggcaaaggagttgaagtttctttctgatgccacgacacaaaatggctctgcctccgctcctagagaaaatgcaaattcagagatcaattga

SEQ ID NO: 40 - DNA, 966 bp, Nepenthes ventricosa
  misc_feature 1..966: note = Nepenthes ventricosa x Nepenthes alata arogenate dehydrogenase betta, partial CDS
  source 1..966: mol_type = other DNA
gccgcgctgccaaacgactacgaaacgaagctttcccatctccctagttctttcgcgaaactcaaggtcgggatcattgggttcggcaattacgggcagttccttgccaaaaccctagtccggcaaggccacaccgttctcgctcattctcgctccaattactcccaaaacgccgcgaagctcggcgtctctttcttctatgatcccaatgacctatgcgaggaacacccggaagttatcctcctctgcacctcgattctgtcgacggaatctgtcctccggagcctgccattgcagcggctcaagcggtctactctcttcgtcgacgttttgtcggtgaaggagtttcctcgatcgcttttgctccaaattctgccccctgacttagacattctctgcactcaccccatgttcgggccggaatccggcaagaacggctggagcgggctgccgttcgtttacgataaggttagaatcggcgaacatgagattagggttaacaggtgtgataattttatcgaagtgttcaggagggaagggtgtaggatggtacagatgagctgtgcggagcacgatcggcatgcggctggctctcagtttataactcatatgatggggagagttttggagaagttgaaattagaggatacgcccattaatacgaaaggctatgagagtttgttgaatttggtggagaacactgcgagggatagtttcgagttgttttatgggctgtttctgtataataagaacgttatggagcagctggagaggatggatttagcgttcgagatggttaaaaagcagttgtttggccatttacatgggttgttgaggagccagttgtttgatggttccgaaatggaagttagagtggaggaggagagaaaattgttgtccgatgggtctcagaatgggcacgttttttcttctttttcagatagtaaaaatgttgagagaaattga

SEQ ID NO: 41 - protein, 53 aa, Beta vulgaris
  REGION 1..53: note = misc_feature - BvADHa N-terminal plastid transit peptide
MISLSSFHPSSTTATATAAAATTHPPQQCPAFSSPPSHLSLPLRHPRQHLVVR

SEQ ID NO: 42 - protein, 74 aa, Beta vulgaris
  REGION 1..74: note = misc_feature - BvADHb N-terminal plastid transit peptide
MLSLSSTTTAKPSPSPSPANFPAKLSSLSTITTTLSFSPRRRYFHGVKTLTIRSIDAAQFFDYESKLAAINTTS

SEQ ID NO: 43 - protein, 398 aa, Beta vulgaris; identical to SEQ ID NO: 2
  REGION 1..398: note = misc_feature - BvADH-alpha Boltardy red beet variety

SEQ ID NO: 44 - DNA, 1197 bp, Beta vulgaris
  misc_feature 1..1197: note = BvADH-alpha Boltardy red beet variety
  source 1..1197: mol_type = other DNA
atgatttcactctcttcttttcatccttcctccaccaccgccaccgccaccgccgccgccgccaccacccacccacctcaacaatgtcccgctttttcctctcctccgtcgcatctctcgcttcctttacgccaccctcgccaacaccttgtagttcggtgcggtggaggtggttcggcctccgaatcggtatttaaccgtgatagtgctgctactcgtgtttctaatgatcatcttgacgttagtaaaagagatgttaagcttaagattgctattattgggtttggtaactttggccagtttttggctaagacaatggctaagcaaggtcatagagtgttggcttactcacgctcggactactcccgcgctgctaaggagatcggcgtcgagtattttactgacgccgatgacctctgcgaggagcaccctgaggttattctgttgtgcacatccatcctctcaacggagaaggtcctccgatcactccccctccaccggctccgtcgttcaaccctctttgcggatgttctctcggtcaaggaatttcctcgatcgctcttccttcaactacttcctaaggactttgatatcctatgcacccaccctatgtttggcccagactcgggcaaagacgggtggggtggactaccttttgtgttcgataaagttagagtcggatcagatcagagtcggacatctcgtgctgaggcattcctagacgtgtttaggaatgccgggtgtaggatggtggaaatgagttgtgttgatcatgacaagcatgcagccggatctcaatttattacacatatgatgggacgagttttggagaaattggccttggaaaatacaccaattaatacaaaagggtacgaaagtttgttaaatttggtggataatactgcaagggatagttttgagttgttttacgggttgtttttgtacaataaaaatgcaatggagcaattggatagaatggattgggctttcgagatggtaaaaaagcaactttcgggatatttgcatgatcttgttagaaaacaattgatgttggagggtaataatgatcaagctgaggttacttttgacaaaccattgatgcttccttctcctactattaatcctccacaaatagttccctctgctgatatggctgagaagaagcatgatttagtggtggttaatggtactagatag

SEQ ID NO: 45 - protein, 323 aa, Mirabilis jalapa
  REGION 1..323: note = misc_feature - Mirabilis jalapa ADH-alpha
IAIVGFGNFGQFLGKEIVKQGHTVLAYSRSDYLRAAHNIGVKFFSDADDLCEEHPQVILLCTSILSTERVLRSLPLHRLRRSTLMVDVLSVKEFPRSLFLQLLPPDFDILCTHPMFGPDSGKAGWGGLPFVFEKVRVGSNPTRSCRVESFLGIFQEAGCRMVEMSCAEHDRHAAGSQFITHMMGRVLEKLALEDTPINTKGYESLLNLVDNTARDSFELFYGLFLYNKNAMEQLDRMHWAFETVKQQLSGYLHDLVRKQLMLESSSNDNNDFVGNYYDNNENDKSSDEKKLMLPAPGVAAAAQILPSSERQQNHDLLYINGRR

SEQ ID NO: 46 - DNA, 972 bp, Mirabilis jalapa
  misc_feature 1..972: note = Mirabilis jalapa ADH-alpha
  source 1..972: mol_type = other DNA
atagcgatagttgggtttggtaactttggtcagtttttgggtaaagaaatagtaaagcaaggtcatactgttttggcttattcacgctctgattacttacgtgctgctcacaacatcggcgtcaaattcttttctgacgccgatgacctttgtgaggaacatcctcaggtgatactgctatgcacatccatcctatcaacagagcgagtccttcgctcactccctctccaccgcctgcgccgctcaacactcatggtagacgtactgtcggtcaaggagtttccccgttcattattccttcaacttctaccaccggactttgacatcctgtgcacacaccccatgtttggacctgactcaggcaaggccgggtggggagggctcccattcgtgtttgaaaaagtgcgagttggatccaacccaacccgctcttgccgggttgagtcctttcttggaatattccaagaagcggggtgtcggatggtggaaatgagttgtgcagaacatgacaggcatgctgcagggtcacagttcataactcacatgatgggtcgtgttttggagaaattagcattagaagacactccaattaacacaaaaggatatgaaagtttactgaatttggttgataacacggcaagagatagctttgagttgttttatggactgtttttgtacaacaagaatgcaatggaacaacttgataggatgcattgggcattcgaaactgttaagcaacagttatctggttacttacacgatctggttcgcaaacaattgatgttagaatcttcaagtaatgataacaatgactttgtcggtaattattatgataataatgaaaatgataagagtagtgatgaaaagaaattgatgcttcctgctcctggagttgcagctgctgctcagattctaccttcttctgaaaggcaacaaaatcatgacttgctctatatcaatggtcgtcgatag

SEQ ID NO: 47 - protein, 386 aa, Beta vulgaris
  REGION 1..386: note = misc_feature - BvADH-beta Boltardy red beet variety
MLSLSSTTTAKPSPSPSPANFPAKLSSLSTITTTLSFSPRRRYFHGVKTLTIRSIDAAQFFDYESKLAAINTTSSSTSSSYSKLKIAIVGFGNYGQFLAKTLVSQGHTVLAYSRSDYSKIAANLGVSYFSDPDDLCEEHPEVIMLCTSILSTEVMLNSLPLQRLKRSTLFVDVLSVKEFPRNLFLQTLPSDFDILCTHPMFGPESGKNGWGSLPFVYDKVRIGKDEGRIKRCESFLDVFRREGCRVEEMTCAEHDKFAAGSQFITHFLGRVLEKLDLEDTPINTKGYESLLNLVDNTSKDSFELFYGLFLYNQNAMEQLERLDWAFELVKKQLFGHLHGLLRKQLFGFSEIDERIGKAKEIKFLSDAAEQNGSALSARENANSETN

SEQ ID NO: 48 - DNA, 1161 bp, Beta vulgaris
  misc_feature 1..1161: note = BvADH-beta Boltardy red beet variety
  source 1..1161: mol_type = other DNA
atgctttctctctcctccacaaccaccgcaaaaccctcgccgtcgccatctccggcgaattttccggcaaaactttcttctctctccaccatcaccaccactctctccttctctcctcgccggagatattttcatggcgtcaaaaccctaacaattcgcagcatcgacgctgcacaattcttcgattacgaatcaaaactcgccgccattaacacaacatcttcatctacatcttcatcttattcgaaactcaaaatcgcaatcgtaggtttcggaaattacggacaatttctggcgaaaaccctagtttctcaaggtcatactgttctcgcttattctcgctctgattactctaaaatcgctgcgaatctcggtgtttcttacttttctgatcctgatgatctttgcgaagaacatcccgaggtaattatgttgtgtacttcgattttatcaactgaagttatgttgaattcgttaccattgcagcgacttaaacgatcgacgctttttgttgatgttttatcggtgaaagaatttccgcgtaatttgtttcttcagactttaccgtctgattttgatatattatgtactcatcctatgtttgggcctgaatctgggaaaaatggttggggaagtttgccgtttgtttatgataaagttaggattgggaaagatgagggtagaattaagagatgtgagagttttttggatgtttttaggagagaaggttgtagggttgaggaaatgacttgtgctgagcatgataagtttgcagcaggatctcagtttataacacatttcttagggagggttttggagaagcttgatttggaggatacgccgattaatacgaaagggtatgagagtttgttgaatttggtggataatacgtcgaaggatagtttcgagttgttttatgggttgtttttgtataatcagaatgctatggagcagttagagaggttagattgggcgtttgagttggttaagaagcaattgtttggacacttgcatgggttgctaaggaaacagttgtttgggttttctgagatagatgaacgtattgggaaggcgaaggagatcaaatttctctctgatgctgcagaacagaatggctctgccttgtctgctagggagaatgcaaattcggagacaaattga

SEQ ID NO: 49 - DNA, 33 bp, synthetic construct
  misc_feature 1..33: note = Synthetic primer
  source 1..33: mol_type = other DNA
ggttccgcgtggatccctaacaattcgcagcat

SEQ ID NO: 50 - DNA, 34 bp, synthetic construct
  misc_feature 1..34: note = Synthetic primer
  source 1..34: mol_type = other DNA
aattcggagacaaattgagaattcatcgtgactg

SEQ ID NO: 51 - DNA, 36 bp, synthetic construct
  misc_feature 1..36: note = Synthetic primer
  source 1..36: mol_type = other DNA
ctggttccgcgtggatcctgcggtggaggtggttcg

SEQ ID NO: 52 - DNA, 35 bp, synthetic construct
  misc_feature 1..35: note = Synthetic primer
  source 1..35: mol_type = other DNA
gttaatggtactagataggaattcatcgtgactga

SEQ ID NO: 53 - DNA, 36 bp, synthetic construct
  misc_feature 1..36: note = Synthetic primer
  source 1..36: mol_type = other DNA
ctggttccgcgtggatccgcaatcgacgccgcccaa

SEQ ID NO: 54 - DNA, 35 bp, synthetic construct
  misc_feature 1..35: note = Synthetic primer
  source 1..35: mol_type = other DNA
tcatcatcatcatcttaagaattcatcgtgactga

SEQ ID NO: 55 - DNA, 36 bp, synthetic construct
  misc_feature 1..36: note = Synthetic primer
  source 1..36: mol_type = other DNA
ctggttccgcgtggatccgccgctaccaatacctcc

SEQ ID NO: 56 - DNA, 35 bp, synthetic construct
  misc_feature 1..35: note = Synthetic primer
  source 1..35: mol_type = other DNA
aattcagagatcaattgagaattcatcgtgactga

SEQ ID NO: 57 - DNA, 36 bp, synthetic construct
  misc_feature 1..36: note = Synthetic primer
  source 1..36: mol_type = other DNA
ctggttccgcgtggatcctgcgccgcctctgactcc

SEQ ID NO: 58 - DNA, 36 bp, synthetic construct
  misc_feature 1..36: note = Synthetic primer
  source 1..36: mol_type = other DNA
tggtaataattctagataggaattcatcgtgactga

SEQ ID NO: 59 - DNA, 37 bp, synthetic construct
  misc_feature 1..37: note = Synthetic primer
  source 1..37: mol_type = other DNA
ctggttccgcgtggatccgccgcgctgccaaacgact

SEQ ID NO: 60 - DNA, 36 bp, synthetic construct
  misc_feature 1..36: note = Synthetic primer
  source 1..36: mol_type = other DNA
aaatgttgagagaaattgagaattcatcgtgactga

SEQ ID NO: 61 - DNA, 37 bp, synthetic construct
  misc_feature 1..37: note = Synthetic primer
  source 1..37: mol_type = other DNA
ctggttccgcgtggatcctgctcatcatcatcatcat

SEQ ID NO: 62 - DNA, 36 bp, synthetic construct
  misc_feature 1..36: note = Synthetic primer
  source 1..36: mol_type = other DNA
cgtcaacgatagatcataggaattcatcgtgactga

SEQ ID NO: 63 - DNA, 37 bp, synthetic construct
  misc_feature 1..37: note = Synthetic primer
  source 1..37: mol_type = other DNA
ctggttccgcgtggatccatagcgatagttgggtttg

SEQ ID NO: 64 - DNA, 36 bp, synthetic construct
  misc_feature 1..36: note = Synthetic primer
  source 1..36: mol_type = other DNA
tatcaatggtcgtcgataggaattcatcgtgactga

SEQ ID NO: 65 - DNA, 38 bp, synthetic construct
  misc_feature 1..38: note = Synthetic primer
  source 1..38: mol_type = other DNA
ctggttccgcgtggatcctgcacggccttcactaaaac

SEQ ID NO: 66 - DNA, 37 bp, synthetic construct
  misc_feature 1..37: note = Synthetic primer
  source 1..37: mol_type = other DNA
tcaatggatcaaagcggtaggaattcatcgtgactga

SEQ ID NO: 67 - DNA, 23 bp, synthetic construct
  misc_feature 1..23: note = Synthetic primer
  source 1..23: mol_type = other DNA
tcaagctgaggttacttttgaca

SEQ ID NO: 68 - DNA, 23 bp, synthetic construct
  misc_feature 1..23: note = Synthetic primer
  source 1..23: mol_type = other DNA
aagaagcatgatttagtggtggt

SEQ ID NO: 69 - DNA, 20 bp, synthetic construct
  misc_feature 1..20: note = Synthetic primer
  source 1..20: mol_type = other DNA
tgcagcgacttaaacgatcg

SEQ ID NO: 70 - DNA, 20 bp, synthetic construct
  misc_feature 1..20: note = Synthetic primer
  source 1..20: mol_type = other DNA
ttggggaagtttgccgtttg

SEQ ID NO: 71 - DNA, 19 bp, synthetic construct
  misc_feature 1..19: note = Synthetic primer
  source 1..19: mol_type = other DNA
agttccctctgctgatatg

SEQ ID NO: 72 - DNA, 21 bp, synthetic construct
  misc_feature 1..21: note = Synthetic primer
  source 1..21: mol_type = other DNA
gtggttaatggtactagatag

SEQ ID NO: 73 - DNA, 20 bp, synthetic construct
  misc_feature 1..20: note = Synthetic primer
  source 1..20: mol_type = other DNA
gcgaaggagatcaaatttct

SEQ ID NO: 74 - DNA, 21 bp, synthetic construct
  misc_feature 1..21: note = Synthetic primer
  source 1..21: mol_type = other DNA
tcaatttgtctccgaatttgc

SEQ ID NO: 75 - DNA, 26 bp, synthetic construct
  misc_feature 1..26: note = Synthetic primer
  source 1..26: mol_type = other DNA
atgatttcactctcttcttttcatcc

SEQ ID NO: 76 - DNA, 30 bp, synthetic construct
  misc_feature 1..30: note = Synthetic primer
  source 1..30: mol_type = other DNA
gatttagtggtggttaatggtactagatag

SEQ ID NO: 77 - DNA, 20 bp, synthetic construct
  misc_feature 1..20: note = Synthetic primer
  source 1..20: mol_type = other DNA
atgctttctctctcctccac

SEQ ID NO: 78 - DNA, 20 bp, synthetic construct
  misc_feature 1..20: note = Synthetic primer
  source 1..20: mol_type = other DNA
caaattcggagacaaattga

SEQ ID NO: 79 - DNA, 20 bp, synthetic construct
  misc_feature 1..20: note = Synthetic primer
  source 1..20: mol_type = other DNA
tctatccttgcatctctcag

SEQ ID NO: 80 - DNA, 20 bp, synthetic construct
  misc_feature 1..20: note = Synthetic primer
  source 1..20: mol_type = other DNA
tctccaagggcgagtatgat

SEQ ID NO: 81 - DNA, 20 bp, synthetic construct
  misc_feature 1..20: note = Synthetic primer
  source 1..20: mol_type = other DNA
cattggttcaggaagtgcaa

SEQ ID NO: 82 - DNA, 20 bp, synthetic construct
  misc_feature 1..20: note = Synthetic primer
  source 1..20: mol_type = other DNA
cctttgattcatggcttcgt

SEQ ID NO: 83 - DNA, 19 bp, synthetic construct
  misc_feature 1..19: note = Synthetic primer
  source 1..19: mol_type = other DNA
tatcaaacgagggcacttc

SEQ ID NO: 84 - DNA, 19 bp, synthetic construct
  misc_feature 1..19: note = Synthetic primer
  source 1..19: mol_type = other DNA
gatggtctttgatagcagc

SEQ ID NO: 85 - DNA, 23 bp, synthetic construct
  misc_feature 1..23: note = Synthetic primer
  source 1..23: mol_type = other DNA
cttttcagtggaattagcccacc

SEQ ID NO: 86 - DNA, 24 bp, synthetic construct
  misc_feature 1..24: note = Synthetic primer
  source 1..24: mol_type = other DNA
tggaacattatggaagatattggg

SEQ ID NO: 87 - DNA, 20 bp, synthetic construct
  misc_feature 1..20: note = Synthetic primer
  source 1..20: mol_type = other DNA
ggctggaagagtgatcggag

SEQ ID NO: 88 - DNA, 22 bp, synthetic construct
  misc_feature 1..22: note = Synthetic primer
  source 1..22: mol_type = other DNA
acgctactgttgagcatcttca

SEQ ID NO: 89 - DNA, 56 bp, synthetic construct
  misc_feature 1..56: note = Synthetic primer
  source 1..56: mol_type = other DNA
gctgtcaacgatacgctacgtaacggcatgacagtgtttttttttttttttttttt

SEQ ID NO: 90 - DNA, 21 bp, synthetic construct
  misc_feature 1..21: note = Synthetic primer
  source 1..21: mol_type = other DNA
agctttacctcccaagtcatc

SEQ ID NO: 91 - DNA, 20 bp, synthetic construct
  misc_feature 1..20: note = Synthetic primer
  source 1..20: mol_type = other DNA
ccaagattgacaggcgttct

SEQ ID NO: 92 - protein, 358 aa, synthetic construct
  REGION 1..358: note = Synthetic: AtADH2
MLLHFSPAKPLISPPNLRRNSPTFLISPPRSLRIRAIDAAQIFDYETQLKSEYRKSSALKIAVLGFGNFGQFLSKTLIRHGHDLITHSRSDYSDAANSIGARFFDNPHDLCEQHPDVVLLCTSILSTESVLRSFPFQRLRRSTLFVDVLSVKEFPKALFIKYLPKEFDILCTHPMFGPESGKHSWSGLPFVYDKVRIGDAASRQERCEKFLRIFENEGCKMVEMSCEKHDYYAAGSQFVTHTMGRVLEKYGVESSPINTKGYETLLDLVENTSSDSFELFYGLFMYNPNALEQLERLDMAFESVKKELFGRLHQQYRKQMFGGEVQSPKKTEQKLLNDGGVVPMNDISSSSSSSSSSS

SEQ ID NO: 93 - protein, 311 aa, synthetic construct
  REGION 1..311: note = Synthetic: AaPDH
MAILSSMFNPSPPQGFCKKNIIKILKSLSMQNVLIVGVGFMGGSFAKSLRRSGFKGKIYGYDINPESISKAVDLGIIDEGTTSIAKVEDFSPDFVMLSSPVRTFREIAKKLSYILSEDATVTDQGSVKGKLVYDLENILGKRFVGGHPIAGTEKSGVEYSLDNLYEGKKVILTPTKKTDKKRLKLVKRVWEDVGGVVEYMSPELHDYVFGVVSHLPHAVAFALVDTLIHMSTPEVDLFKYPGGGFKDFTRIAKSDPIMWRDIFLENKENVMKAIEGFEKSLNHLKELIVREAEEELVEYLKEVKIKRMEID

SEQ ID NO: 94 - protein, 279 aa, synthetic construct
  REGION 1..279: note = Synthetic: SyADH
MKIGVVGLGLIGASLAGDLRRRGHYLIGVSRQQSTCEKAVERQLVDEAGQDLSLLQTAKIIFLCTPIQLILPTLEKLIPHLSPTAIVTDVASVKTAIAEPASQLWSGFIGGHPMAGTAAQGIDGAEENLFVNAPYVLTPTEYTDPEQLACLRSVLEPLGVKIYLCTPADHDQAVAWISHLPVMVSAALIQACAGEKDGDILKLAQNLASSGFRDTSRVGGGNPELGTMMATYNQRALLKSLQDYRQHLDQLITLISNQQWPELHRLLQQTNGDRDKYVE

(19) United States
(12) Patent Application Publication
(10) Pub. No.: US 2026/0132413 A1
(43) Pub. Date: May 14, 2026

(54) AROGENATE DEHYDROGENASE POLYNUCLEOTIDES, POLYPEPTIDES AND METHODS OF USING THE SAME

(71) Applicant: Wisconsin Alumni Research Foundation, Madison, WI (US)

(72) Inventors: Hiroshi A. Maeda, Madison, WI (US); Samuel Lopez-Nieves, Madison, WI (US); Marcos Viana de Oliveira, Madison, WI (US)

(21) Appl. No.: 19/286,041

(22) Filed: Jul. 30, 2025

Related U.S. Application Data
(63) Continuation of application No. 15/932,337, filed on Feb. 16, 2018, now Pat. No. 12,378,567.
(60) Provisional application No. 62/459,798, filed on Feb. 16, 2017.

(51) Int. Cl. C12N 15/82; A01H 5/00; C07H 21/04; C12N 15/00; C12P 13/22

(52) U.S. Cl. CPC C12N 15/8251; A01H 5/00; C07H 21/04; C12N 15/00; C12P 13/22; C12Y 103/01043

(57) ABSTRACT

The invention generally relates to arogenate dehydrogenase polynucleotides and methods of using the same. More specifically, the invention relates in part to compositions including arogenate dehydrogenase polynucleotides from beet varieties and other Caryophyllales species and methods of using the same.

### CROSS-REFERENCE TO RELATED PATENT APPLICATIONS

The present application is a continuation of U.S. application Ser. No. 15/932,337, led Feb. 16, 2018, which application claims the benefit of priority to U.S. Provisional Patent Application No. 62/459,798, led on Feb. 16, 2017, the contents of which are incorporated herein by reference in their entireties.

### STATEMENT REGARDING FEDERALLY SPONSORED RESEARCH

This invention was made with United States government support under grant number 2015-67013-22955 awarded by the US Department of Agriculture, National Institute of Food and Agriculture. The government has certain rights in this invention.

### SEQUENCE LISTING

A Sequence Listing accompanies this application and is submitted as an XML file of the sequence listing named “96029604722_SL.xml” which is 155,202 bytes in size and was created on Jan. 26, 2026. The sequence listing is electronically submitted via Patent Center and is incorporated herein by reference in its entirety.

### INTRODUCTION

Plants synthesize numerous specialized metabolites (also known as secondary metabolites), which play crucial roles in plant adaptation. In contrast to well-documented diversification of plant enzymes directly involved in specialized metabolism, relatively little is known about the evolution of primary metabolic enzymes that provide precursors to the production of various specialized metabolites.

L-Tyrosine (Tyr) is an aromatic amino acid required for protein biosynthesis in all organisms; however, it is synthesized de novo only in bacteria, fungi and plants, but not in animals. Consequently, animals have to consume Tyr, or L-phenylalanine (Phe) that can be hydroxylated to Tyr. Besides protein biosynthesis, plants also use Tyr to produce a diverse array of specialized metabolites that are important for defense (e.g. dhurrin), antioxidants (e.g. tocopherols), and pollinator attraction (e.g., betalains). Notably, humans have a long history of utilizing Tyr-derived specialized metabolites, such as the psychedelic alkaloid mescaline derived from the cactus Lophophora williamsii and the analgesic morphine derived from Papaver somniferum (oppium poppy).

Tyr is synthesized from prephenate, which is converted from the final product of the shikimate pathway, chorismate. In most bacteria and fungi, prephenate is oxidatively decarboxylated by prephenate dehydrogenase (TyrAₚ/PDH, hereafter referred only as PDH; EC 1.3.1.12) to produce 4-hydroxyphenylpyruvate (HPP), which is subsequently transaminated to Tyr (See, e.g., FIG. 1). On the other hand, most plants first transaminate prephenate into arogenate and subsequently decarboxylate into Tyr by arogenate dehydrogenase (TyrAₐ/ADH, hereafter referred only as ADH; EC 1.3.1.78), both steps occurring in the plastids. The Tyr pathway is usually highly regulated at PDH and ADH. These homologous enzymes are strongly feedback inhibited by Tyr and control carbon flow between the two competing Tyr and Phe pathways. A recent report showed that, in addition to plastidic ADH enzymes, some plants possess a PDH enzyme(s) that is not inhibited by Tyr and is localized to the cytosol. Clearly, there is evolutionary variation in the Tyr pathway(s) in different plant lineages that warrants investigation. In addition, the contribution of Tyr biosynthesis and its regulation to the generation of Tyr-derived plant natural products is currently unknown.

Betalains are a class of pigments that, within the flowering plants, occur exclusively in the order Caryophyllales where they replace the otherwise ubiquitous anthocyanins. Within Caryophyllales, the majority of families are betalain pigmented. In two families, Molluginaceae and Caryophyllaceae, however, evolutionary reversions from betalain to anthocyanin pigmentation have occurred, highlighting the fact that these two classes of water-soluble pigments have never been found in the same organism. Betalains and anthocyanins are synthesized from Tyr and Phe, respectively, but have similar chemical properties and physiological functions in pollinator attraction and stress tolerance. Betalains are also used as a natural food dye (E162) and have anticancer and antidiabetic properties. Furthermore, intermediates in the betalain pathway are important pharmaceuticals [e.g. L-dihydroxyphenylalanine (L-DOPA) for the treatment of Parkinson's disease) or are substrates for other pharmaceutical agents (e.g. the production of dopamine and isoquinoline alkaloids such as morphine). Consequently, understanding the coordinated regulation of Tyr and betalain biosynthesis has the potential to enhance the production of Tyr, and the yield of Tyr-derived plant natural products important for human health and nutrition.

### SUMMARY

In one aspect, ADH polynucleotides encoding ADH polypeptides are provided. The polynucleotides may encode a polypeptide having at least 50%, 55%, 60%, 65%, 70%, 75%, 80%, 85%, 90%, 95%, or 99% sequence identity to any one of the polypeptides of SEQ ID NOS: 1-20, 43, 45, or 47. SEQ ID NOS: 1-20, 43, 45, or 47 are polypeptide sequences of ADHα and ADHβ polypeptides identified in W357B red beet variety, Big Buck sugar beet variety, Touch Stone yellow beet variety, Blankoma white beet variety, Sea beet PI562585 variety, and other Caryophyllales species.

In another aspect, constructs are provided. The constructs may include a heterologous promoter operably linked to any one of the polynucleotides described herein.

In a further aspect, vectors including any of the constructs or polynucleotides described herein are provided.

In another aspect, cells including any of the polynucleotides, constructs, or vectors described herein are provided.

In a further aspect, plants including any of the polynucleotides, constructs, vectors, or cells described herein are also provided.

In a still further aspect, methods for increasing production of at least one product of the tyrosine or HPP pathways in a cell are provided. The methods may include introducing any of the polynucleotides, constructs, or vectors described herein into the cell. Optionally, the methods may further include purifying the product of the tyrosine or HPP pathways from the cells.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIGS. 1A-1D shows Beta vulgaris have two ADH enzymes localized in the plastids. FIG. 1A shows tyrosine and betalain biosynthetic pathways in plants. L-Tyrosine (Tyr) can be synthesized from prephenate via arogenate dehydrogenase (ADH/TyrAₐ) or prephenate dehydrogenase (PDH/TyrAₚ). Tyr is exported from the plastid to cytosol and then converted to L-dihydroxyphenylalanine (L-DOPA) by CYP76AD1α, CYP76AD5, and CYP76AD6 (CYP76AD1α/5/6). L-DOPA is then eventually converted to betalains, red betacyanins and yellow betaxanthins. Biosynthesis of Tyr competes for arogenate or prephenate substrate with that of L-phenylalanine (Phe), the precursor of anthocyanins. Dashed lines denote feedback regulation by Tyr. DODA, L-DOPA dioxygenase. FIG. 1B is a graph showing arogenate substrate was incubated with the purified recombinant enzymes of BvADHα or BvADHβ together with NADP⁺ cofactor and the production of Tyr was analyzed. The High Pressure Liquid Chromatography (HPLC) traces were offset for presentation. Arabidopsis thaliana ADH2 (AtADH2) was used as a control for the ADH assay. In FIG. 1C green fluorescence protein (GFP) was fused at the C-terminal of BvADHα and BvADHβ and transiently expressed in Arabidopsis protoplasts. Free GFP and GFP-fused Arabidopsis ADH2 (AtADH2) were used as controls for cytosolic and plastidic localization, respectively. Representative images show GFP fluorescence and chlorophyll autofluorescence. Scale bars, 10 μm. FIG. 1D is a set of graphs showing expression levels of BvADHα and BvADHβ were compared with those of betalain pathway genes in the cotyledon and hypocotyl of 7 day-old sugar beet and red beet (W357B). Asterisks indicate significant differences between the two genotypes (p<0.05, Student's t-test). Bars represent percent expression relative to the sample with the highest expression. Data are means of three biological replicates ±s.e.m. N.D., not detectable.

FIGS. 2A-2B show physical location, homology, and phylogeny of BvADHα and BvADHβ. FIG. 2A shows the location and physical distance of BvADHα and BvADHβ on chromosome 8 of the B. vulgaris genome. FIG. 2B shows amino acid identity of ADH and PDH proteins from different plants and bacteria. AaPDH, Aquifexaeolicus; AtADH1 and AtADH2, Arabidopsis thaliana; GmPDH1, Glycine max; EcPDH, Escherichia coli; and SyADH, Synechocystis sp. PCC6803.

FIGS. 3A-3C shows ADH but not PDH activity detected from B. vulgaris tissues (FIGS. 3A, 3B) or recombinant enzyme (FIG. 3C). Arogenate (FIG. 3A) or prephenate (FIGS. 3B, 3C) substrates were incubated with NADP⁺ cofactor and desalted protein crude extract (FIGS. 3A, 3B) of beet leaf (L), root/stem (R/S) tissues or recombinant enzyme of BvADHα or BvADHβ together with NADP⁺ cofactor (FIG. 3C). The production of Tyr (FIG. 3A) or HPP (which was converted to 4-hydroxyphyenyllactic acid, HPLA) FIGS. 3B, 3C were analyzed by HPLC. The HPLC traces were offset for presentation. Arabidopsis thaliana ADH2 (AtADH2) [17,18] and Medicago truncatula PDH (MtPDH) [22] were used as a control for the ADH and PDH assay, respectively.

FIG. 4 shows BvADHs prefer NADP⁺ over NAD⁺ as cofactor. ADH activity was analyzed using NADP⁺ or NADP⁺ cofactor, which is expressed as the mean of three independent experiments ±s.e.m. in nmols-1 mg-1 of protein.

FIGS. 5A1-5D show no amino acid changes were found in the mature protein coding region of BvADHα among different B. vulgaris varieties. The BvADHα and BvADHβ genes were sequenced from five different varieties of domesticated (red 1 [W357B], red 2 [Boltardy], sugar, yellow, and white) and a wild beet (sea beet ascension number PI562585). In nucleotide sequence comparisons of BvADHα (FIG. 5A, SEQ ID NOs: 21-25, 44) and BvADHβ (FIG. 5B SEQ ID NOs: 34-38, 48), several single nucleotide polymorphisms (SNPs) were found among varieties. Amino acid sequence alignments of BvADHα (FIG. 5C, SEQ ID NOs: 1-5, 43) and BvADHβ (FIG. 5D, SEQ ID NOs: 14-18, 47), however, showed that these SNPs were mostly synonymous (no changes in amino acid), with two exceptions found in the N-terminal predicted chloroplast transit peptide, which was eliminated for recombinant enzyme expression. The predicted chloroplast transit peptide cleavage sites are denoted by triangles.

FIG. 6 shows beet and spinach ADHα but not ADHβ have reduced sensitivity to Tyr. ADH activity was measured at different Tyr concentrations using NADP⁺ cofactor and purified recombinant ADH enzymes of beet (BvADHα, BvADHβ), spinach (SoADHα, SoADHβ), and Arabidopsis (AtADH2). Data are expressed as the percentage of respective control activity without Tyr (0 μM) and means of three independent experiments ±s.e.m. N.D., not detectable; N.T., not tested.

FIG. 7 shows recombinant His-tagged BvADHα also exhibits reduced sensitivity to Tyr relative to AtADH2. BvADHα and AtADH2 recombinant enzymes were also generated as 6× His-tag proteins to determine if GST-tag affects Tyr sensitivity of BvADHα. The His-BvADHα recombinant enzyme still exhibited relaxed sensitive to Tyr inhibition. Data are expressed as the percentage of respective control activity without Tyr (0 μM) and the means of three independent experiments s.e.m. N.D., not detectable; N.T., not tested.

FIG. 8 shows BvADHs are not inhibited by phenylalanine, tryptophan, and betanin. ADH activity of BvADHα, BvADHβ and AtADH2 was measured in the presence and absence of 1 mM final concentration of L-phenylalanine (L-Phe), L-tryptophan (L-Trp), and betanins as an effector.

Data are expressed as the percentage of respective control activity without effector and the mean of three independent experiments s.m.e. No significant reduction was observed by any effector treatment relative to respective no effector control (P<0.05, student t test).

FIGS. 9A-9B show transgene expression and tyrosine levels of individual leaf samples of infiltrated Nicotiana benthamiana. Agrobacterium tumefaciens carrying the construct of 35S::GFP, 35S::BvADHα, or 35S::BvADHfl was infiltrated to Nicotiana benthamiana leaves (sample names ending with G, a, and b, respectively). 1a-1 and 1a-2 are technical replicates of the same leaf infiltrated with 35S::BvADHα, so do 1b-1 and 1b-2 for 35S::BvADHβ. FIG. 9A shows expression of respective transgenes shown by RTPCR. (+) denotes a positive control using the original plasmid as a template, while (−) indicates a negative control cDNA from a leaf area without infiltration. (−RT) is an additional negative control without reverse transcriptase to detect genomic DNA contamination. FIG. 9B shows tyrosine contents of individual samples. Two technical replicates showed very similar results. Means±s.e.m. of Tyr and other amino acids analysis are shown in FIGS. 10A-10B and Table 2.

FIGS. 10A-10B shows heterologous expression of BvADHα but not BvADHβ increases tyrosine levels in Nicotiana benthamiana. Agrobacterium tumefaciens carrying the construct of 35S::GFP, 35S::BvADHα, or 35S::BvADHf was infiltrated to N. benthamiana leaves, which were analyzed for amino acid contents using GC-MS. The levels of tyrosine (FIG. 10A) and phenylalanine (FIG. 10B) are shown. Asterisks indicate significant differences from the 35S::GFP control (p<0.05, Student's t-test). Data are means S.E.M. (n=5).

FIGS. 11A-11C show phylogenetic distribution of ADHα in Caryophyllales. FIG. 11A shows maximum-likelihood phylogeny of ADH genes in Caryophyllales. The blue and pink branches represent anthocyanin and betalain-producing families, respectively, while families with unclear/unidentified pigmentation are shown in gray. Scale bar indicates inferred number of amino acid substitution per site. ADH enzymes characterized in this study are indicated at the end of each branch. FIG. 11B shows presence and absence of BvADHα and BvADHβ orthologs detected from genome or transcriptome data was mapped to the family-level phylogenetic tree of the Caryophyllales order. Filled circles denote that corresponding orthologs were detected in all species within the family, whereas partially filled circles indicate that the filled portion of the species within each family had corresponding orthologs. Open circles denote no corresponding orthologs were detected. Dark lines on the left, labeled “ADHα/β” and “CYP76AD1α/β DODAα/β,” indicate estimating timings of duplication events of ADH and betalain pathway genes (CYP76AD1 and DODA). Dash lines (−) represent families with no available transcriptomic or genomic data. FIG. 11C shows Tyr contents analyzed in various Caryophyllales species. Arabidopsis thaliana was used as outgroup. Young leaf tissues were used for all samples except a Cactaceae species, in which flowers were used to avoid succulent tissues. Asterisks denote significant difference from Arabidopsis (p<0.05) based on fixed effect model (see method). Also, a statistical analysis based on the mixed effect model showed significant differences between two groups, plants with and without ADHα (p<0.0001). Bars represent means±s.e.m. (n=four biological replicates).

FIG. 12 shows ADHα from various species of core Caryophyllales also exhibit relaxed sensitivity. ADH activity was measured under different Tyr concentrations using purified recombinant ADH enzymes of Nepenthes ventricosa x alata (NaADHβ), Rivina humilis (RhADHα), Mirabilis jalapa (MjADHα), and Portulaca oleracea (PoADHα) ADH. Data are expressed as the percentage of respective control activity without Tyr (0 μM) and the mean of three independent experiments ±s.e.m. N.D., not detectable; N.T., not tested.

FIG. 13 shows Tyr sensitivity of ADH activity from plant tissues. The plastid extracts of spinach (Spinaciaoleracea), and the crude extracts of Dianthus barbatus and Arabidopsis thaliana were incubated with 1 m Marogenate substrate and 1 mM NADP+ cofactor for indicated times. Plastids were isolated for spinach ADH assays to eliminate strong polyphenoloxidase activity present in the crude extracts. Data are means±s.e.m. (n=4). Activity increased linearly during the first two hours, which were used to calculate ADH activity presented in Table 4.

FIGS. 14A-14B shows ADHα sequences used for texting relax selection. FIGS. 14A and 14B show ADHα orthologs of Caryophyllaceae (branches below the dashed lines, designated as test branches in RELAX analysis, Table 5), as compared to those betalain-producing Caryophyllales species (branches above the dashed lines, designated as reference branches in RELAX analysis, Table 5). The test branches showed no obvious acceleration of substitution in their coding sequences (CDS, FIG. 14A), whereas there was apparent acceleration in their peptide sequences (FIG. 14B). Tips marked with ‘@’ are from assembled transcriptomes. The rest of the sequences are from PCR and Sanger sequencing from DNA (H. latifolia, S. marina, and P. polygonfolia) or RNA.

FIG. 15 shows the Histidine 217 residue responsible for Tyr sensitivity of Aquifex aeolicus PDH (AaPDH) is still present in BvADHα. Previous studies showed that the H217 residue of AaPDH (denoted by triangles) is absent in Tyr-insensitive ADH of Synechocystis sp. PCC6803 (SyADH) and confers Tyr sensitivity of AaPDH (Sun et al., 2009, Legrand, P. et al. 2008). The amino acid alignment of AaPDH, SyADH together with BvADHα, BvADHβ, and Arabidopsis ADH (AtADH2) (SEQ ID NOs: 1, 14, and 92-94) showed that corresponding His residues are present in all plant ADHs. This result suggests that yet to be identified novel residues and mechanism are involved in the relaxed Tyr sensitivity of BvADHα.

FIG. 16 shows expression of BvADHα in Arabidopsis leads to hyper-accumulation of tyrosine. Overexpression of tyrosine-insensitive BvADHα, but not BvADHβ or AtADH2, in Arabidopsis drastically enhanced accumulation of tyrosine and homogentisate, the downstream product of tyrosine and precursor of tocopherols and plastoquinone. Four-week old Arabidopsis leaf tissue was submitted to chemical analysis by GC-MS. Two representative homozygous lines for each construct were selected. Control plants (Ctrl) are lines transformed with the empty vector. The content of tyrosine (Tyr), homogentisate, phenylalanine (Phe), and alanine (Ala) are shown as nmol/g of fresh weight. Samples were normalized by the internal recovery standard, norvaline. Values are mean of 3 biological replicates ±SD (standard deviation). The above experiments were repeated at least 3 times with similar results.

FIG. 17 shows in planta expression of de-regulated BvADHα leads to enhanced accumulation of Tyr in Arabidopsis.

FIG. 18 shows heterologous expression of de-regulated BvADHα leads to hyper-accumulation of Tyr in Glycine max (soybean).

### DETAILED DESCRIPTION

The present inventors investigated the Tyr biosynthetic pathway and its regulation in table beet (Beta vulgaris L.), which produces high levels of betalains. Using comparative genomics, biochemical, and cellular analyses, they found that B. vulgaris possesses two paralogous genes encoding two ADH enzymes, which they named ADHα and ADHβ. Interestingly, ADHα but not ADHβ exhibited relaxed sensitivity to Tyr inhibition. Although the present inventors recently reported that legume PDH enzymes are also Tyr insensitive, BvADHα and legume PDHs have two major differences. First, legume PDHs are localized in the cytosol, whereas BvADHα (and BvADHβ) was targeted to the plastids. Second, legume PDHs completely lost Tyr sensitivity but BvADHα was still inhibited by Tyr at higher concentrations.

Other insensitive ADH/PDH enzymes have been previously found in microorganisms and the structural analyses of Tyr sensitive and insensitive enzymes identified histidine 217 as a possible residue responsible for its Tyr sensitivity. However, the corresponding histidine residue was still present in BvADHα, suggesting that different mechanisms, and as yet unidentified residues, are involved in the relaxed Tyr sensitivity of BvADHα. The identified BvADHα and other Caryophyllales ADHα enzymes may be introduced into various types of cells to deregulate Tyr biosynthesis and redirect carbon flow from Phe to Tyr, to improve the production of Tyr-derived products (e.g., vitamin E, isoquinoline alkaloids including morphine).

ADH polynucleotides encoding ADH polypeptides are provided. The polynucleotides may encode a polypeptide having at least 50%, 55%, 60%, 65%, 70%, 75%, 80%, 85%, 90%, 95%, or 99% sequence identity to any one of the polypeptides of SEQ ID NOS: 1-20, 43, 45, or 47. SEQ ID NOS: 1-20, 43, 45, or 47 are polypeptide sequences of ADHα and ADHβ polypeptides identified in W357B red beet variety, Big Buck sugar beet variety, Touch Stone yellow beet variety, Blankoma white beet variety, Sea beet PI562585 variety, and other Caryophyllales species.

As used herein, the terms “polynucleotide,” “polynucleotide sequence,” “nucleic acid” and “nucleic acid sequence” refer to a nucleotide, oligonucleotide, polynucleotide (which terms may be used interchangeably), or any fragment thereof. These phrases also refer to DNA or RNA of natural or synthetic origin (which may be single-stranded or double-stranded and may represent the sense or the antisense strand). The polynucleotides may be cDNA or genomic DNA.

In some embodiments, the polynucleotides of the present invention may include any one of the polynucleotide sequences of SEQ ID NOS: 21-40, 44, 46, or 48 or a polynucleotide having at least 50%, 55%, 60%, 65%, 70%, 75%, 80%, 85%, 90%, 95%, or 99% sequence identity to any one of the polynucleotide sequences of SEQ ID NOs: 21-40, 44, 46, or 48. SEQ ID NOS: 21-40, 44, 46, or 48 are polynucleotide sequences of ADHα and ADHβ polynucleotides that encode the ADHα and ADHβ polypeptides of SEQ ID NOS: 1-20, 43, 45, or 47 and identified in W357B red beet variety, Big Buck sugar beet variety, Touch Stone yellow beet variety, Blankoma white beet variety, Sea beet PI562585 variety, and other plant species. The polynucleotide sequences of SEQ ID NO: 21-40, 44, 46, or 48 are cDNA sequences.

Polynucleotides homologous to the polynucleotides described herein are also provided. Those of skill in the art understand the degeneracy of the genetic code and that a variety of polynucleotides can encode the same polypeptide. In some embodiments, the polynucleotides (i.e., polynucleotides encoding the ADH polypeptides) may be codon-optimized for expression in a particular cell including, without limitation, a plant cell, bacterial cell, or fungal cell. While particular polynucleotide sequences which are found in plants are disclosed herein any polynucleotide sequences may be used which encode a desired form of the polypeptides described herein. Thus, non-naturally occurring sequences may be used. These may be desirable, for example, to enhance expression in heterologous expression systems of polypeptides or proteins. Computer programs for generating degenerate coding sequences are available and can be used for this purpose. Pencil, paper, the genetic code, and a human hand can also be used to generate degenerate coding sequences.

Regarding ADH polypeptides, the phrases “% sequence identity,” “percent identity,” or “% identity” refer to the percentage of residue matches between at least two amino acid sequences aligned using a standardized algorithm. Methods of amino acid sequence alignment are well-known. Some alignment methods take into account conservative amino acid substitutions. Such conservative substitutions, explained in more detail below, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastp,” that is used to align a known amino acid sequence with other amino acids sequences from a variety of databases.

Polypeptide sequence identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

Suitably, the polypeptides encoded by the polynucleotides provided herein are not sensitive to tyrosine inhibition. The polypeptide is considered to not be sensitive, i.e. to lack sensitivity to tyrosine feedback inhibition, if at least 50% of the activity in the absence of tyrosine is maintained in the presence of 1-100 μM (or any range therein) tyrosine. The polypeptide is considered to lack tyrosine feedback sensitivity if at least 40% of the activity in the absence of tyrosine is maintained in the presence of 1 mM tyrosine.

The ADH polypeptides disclosed herein may include “variant” polypeptides, “mutants,” and “derivatives thereof.” As used herein the term “wild-type” is a term of the art understood by skilled persons and means the typical form of a polypeptide as it occurs in nature as distinguished from variant or mutant forms. As used herein, a “variant, “mutant,” or “derivative” refers to a polypeptide molecule having an amino acid sequence that differs from a reference protein or polypeptide molecule. A variant or mutant may have one or more insertions, deletions, or substitutions of an amino acid residue relative to a reference molecule. For example, a ADH polypeptide mutant or variant may have one or more insertions, deletions, or substitution of at least one amino acid residue relative to the ADH “wild-type” polypeptides disclosed herein. The polypeptide sequences of the “wild-type” ADH polypeptides from beets and other plant species are presented in SEQ ID NOS: 1-20, 43, 45, or 47. These sequences may be used as reference sequences.

The ADH polypeptides provided herein may be full-length polypeptides or may be fragments of the full-length polypeptide. As used herein, a “fragment” is a portion of an amino acid sequence which is identical in sequence to but shorter in length than a reference sequence. A fragment may comprise up to the entire length of the reference sequence, minus at least one amino acid residue. For example, a fragment may comprise from 5 to 1000 contiguous amino acid residues of a reference polypeptide, respectively. In some embodiments, a fragment may comprise at least 5, 10, 15, 20, 25, 30, 40, 50, 60, 70, 80, 90, 100, 150, 250, or 500 contiguous amino acid residues of a reference polypeptide. Fragments may be preferentially selected from certain regions of a molecule. The term “at least a fragment” encompasses the full length polypeptide. A fragment of an ADH polypeptide may comprise or consist essentially of a contiguous portion of an amino acid sequence of the full-length ADH polypeptide (See SEQ ID NOS: 1-20, 43, 45, or 47). A fragment may include an N-terminal truncation, a C-terminal truncation, or both truncations relative to the full-length ADH polypeptide.

A “deletion” in an ADH polypeptide refers to a change in the amino acid sequence resulting in the absence of one or more amino acid residues. A deletion may remove at least 1, 2, 3, 4, 5, 10, 20, 50, 100, 200, or more amino acids residues. A deletion may include an internal deletion and/or a terminal deletion (e.g., an N-terminal truncation, a C-terminal truncation or both of a reference polypeptide).

“Insertions” and “additions” in an ADH polypeptide refer to changes in an amino acid sequence resulting in the addition of one or more amino acid residues. An insertion or addition may refer to 1, 2, 3, 4, 5, 10, 20, 30, 40, 50, 60, 70, 80, 90, 100, 150, 200, or more amino acid residues. A variant of an ADH polypeptide may have N-terminal insertions, C-terminal insertions, internal insertions, or any combination of N-terminal insertions, C-terminal insertions, and internal insertions.

The amino acid sequences of the ADH polypeptide variants, mutants, derivatives, or fragments as contemplated herein may include conservative amino acid substitutions relative to a reference amino acid sequence. For example, a variant, mutant, derivative, or fragment polypeptide may include conservative amino acid substitutions relative to a reference molecule. “Conservative amino acid substitutions” are those substitutions that are a substitution of an amino acid for a different amino acid where the substitution is predicted to interfere least with the properties of the reference polypeptide. In other words, conservative amino acid substitutions substantially conserve the structure and the function of the reference polypeptide. Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

The disclosed variant and fragment ADH polypeptides described herein may have one or more functional or biological activities exhibited by a reference polypeptide (e.g., one or more functional or biological activities exhibited by wild-type ADH polypeptides (i.e, SEQ ID NOS: 1-20, 43, 45, or 47). Suitably, the disclosed variant or fragment ADH polypeptides retain at least 20%, 40%, 60%, 80%, or 100% of the arogenate dehydrogenase activity of the reference polypeptide (i.e., SEQ ID NOS: 1-20, 43, 45, or 47). As used herein, a “functional fragment” of an ADH polypeptide is a fragment of, for example, one of the polypeptides of SEQ ID NOS: 1-20 that retains at least 20%, 40%, 60%, 80%, or 100% of the arogenate dehydrogenase activity of the full-length ADH polypeptide. Exemplary functional fragments of the ADH polypeptides disclosed herein may include, for example, fragment ADH polypeptides of the polypeptides of SEQ ID NOS: 1-20 that lack the N-terminal plastid transit peptide within these sequences. The N-terminal plastid transit peptide (identified by SEQ ID NO: 41 for BvADHα and SEQ ID NO: 42 for BvADHR) functions to localize the ADH polypeptides of SEQ ID NOS: 1-20, 43, 45, or 47 to the plastid in plant cells. This function is not necessarily required for the ADH polypeptides arogenate dehydrogenase activity and thus may be removed from SEQ ID NOS: 1-20, 43, 45, or 47.

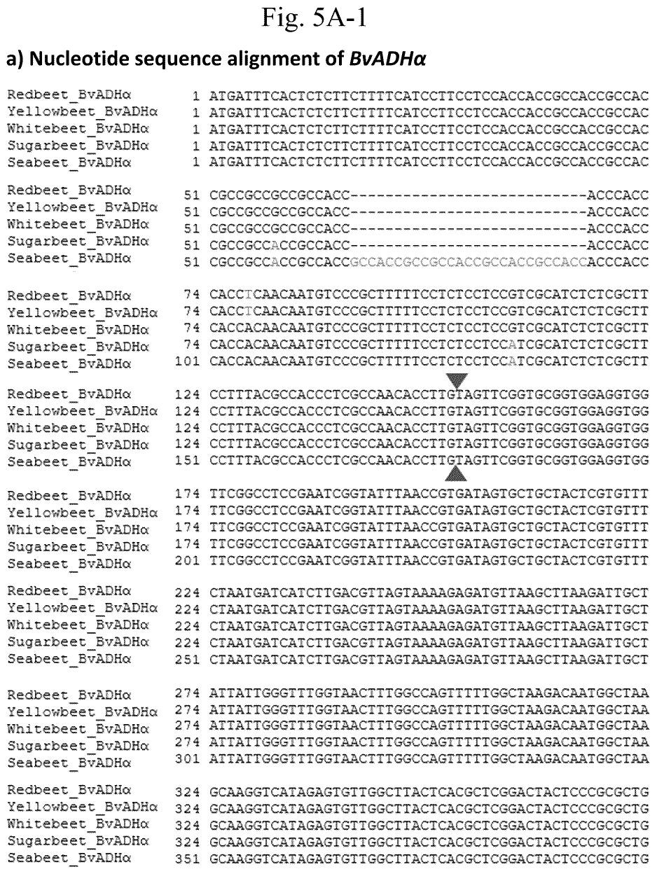

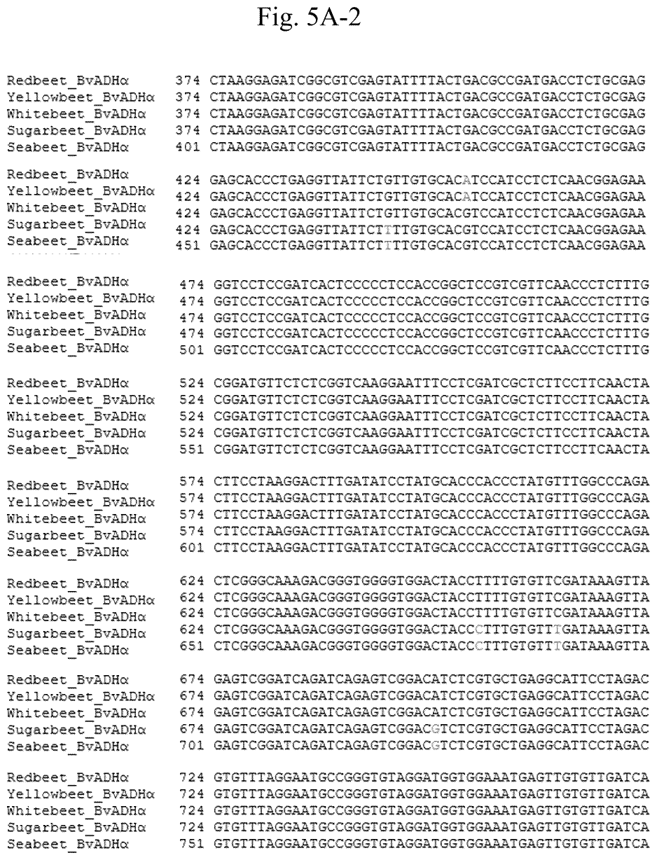

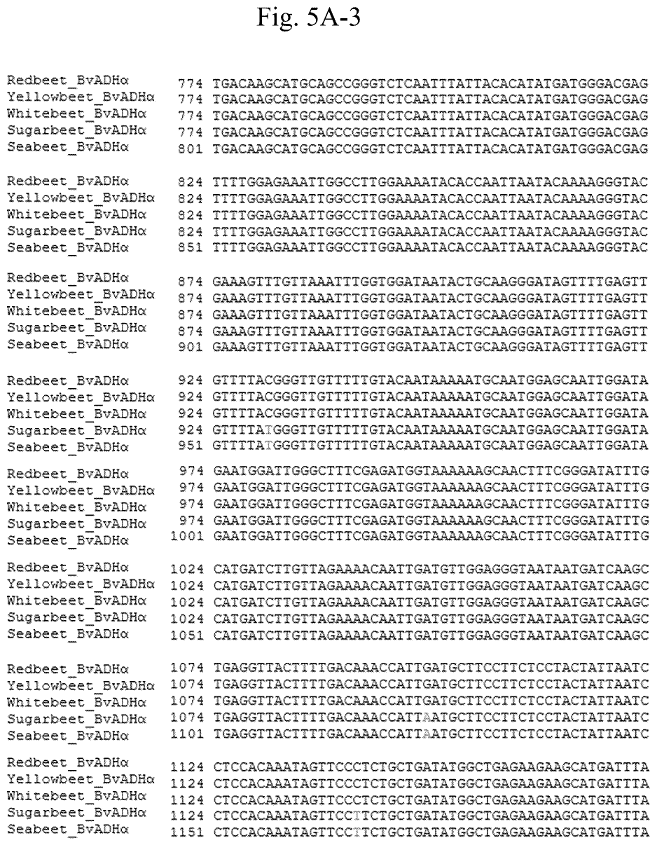

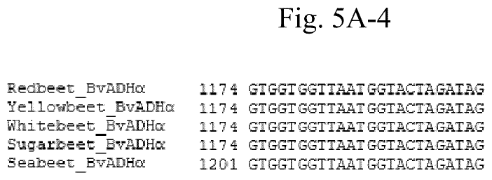

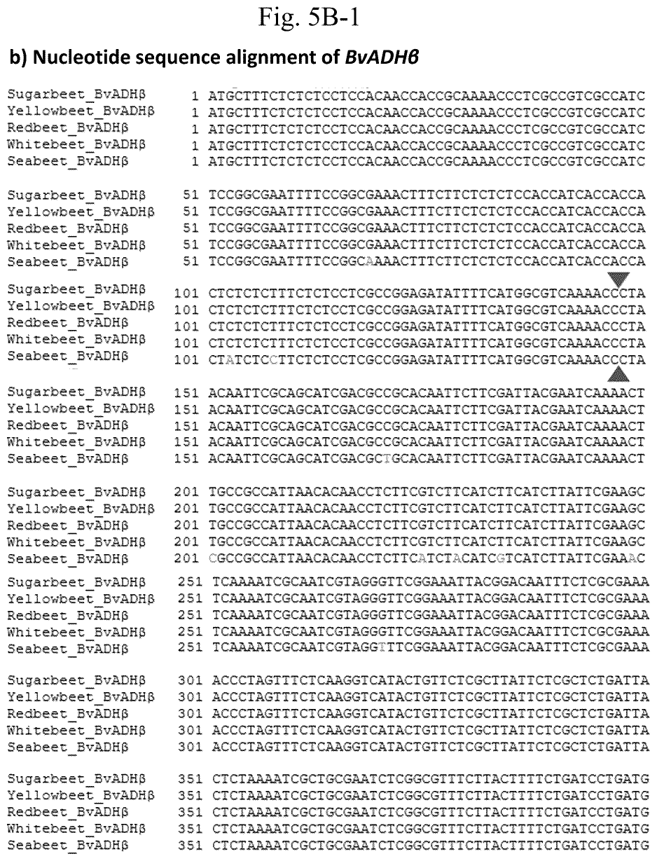

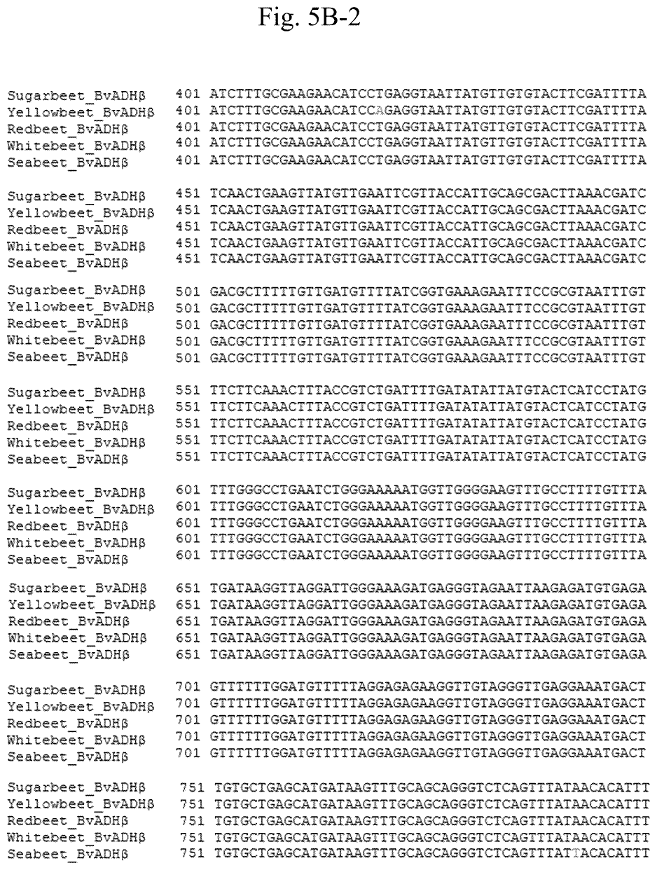

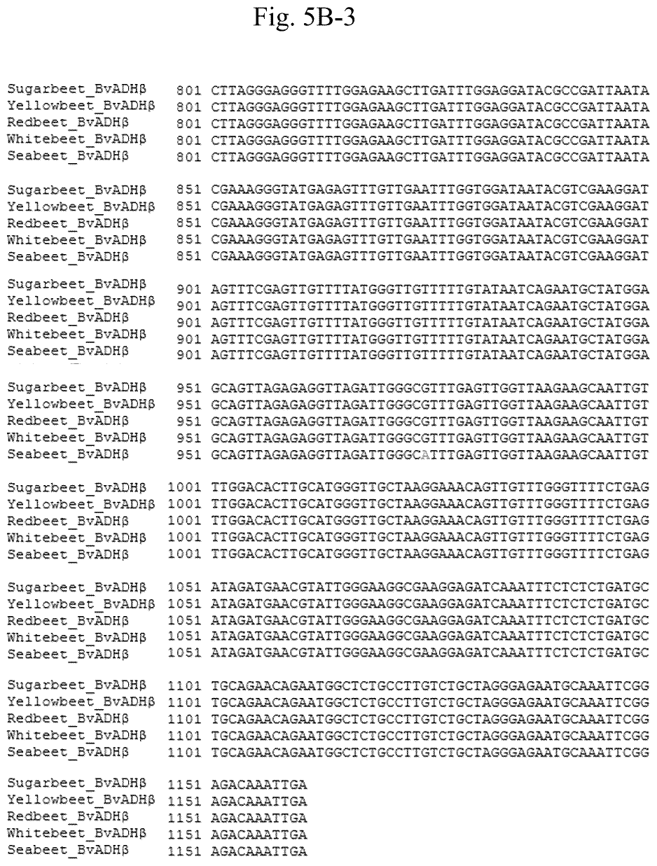

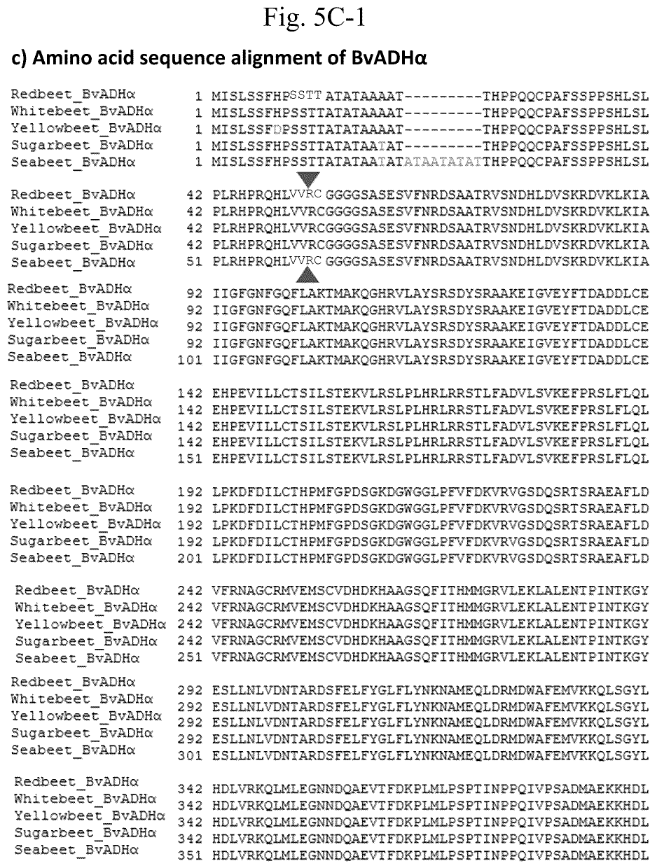

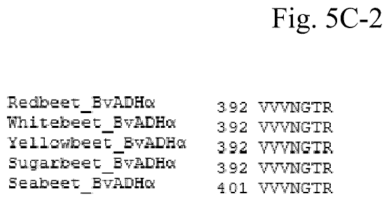

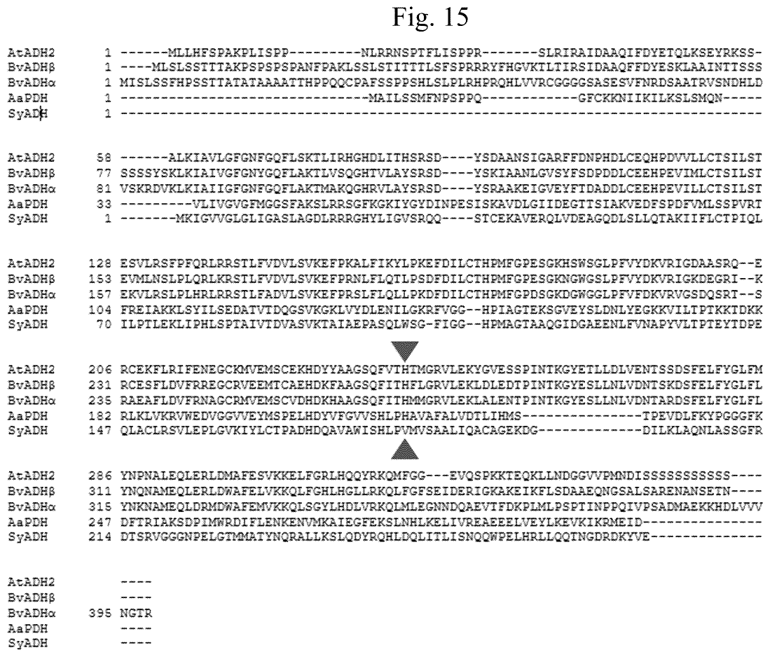

FIGS. 5A1-5D and FIG. 15 show sequence alignments including some of the ADH polypeptides disclosed as SEQ ID NOs: 1-20. Based on these alignments it becomes immediately apparent to a person of ordinary skill in the art that various amino acid residues may be altered (i.e, substituted, deleted, etc.) without substantially affecting the arogenate dehydrogenase activity of the polypeptide. For example, a person of ordinary skill in the art would appreciate that substitutions in a reference ADH polypeptide could be based on alternative amino acid residues that occur at the corresponding position in other ADH polypeptides from other species. SEQ ID NOS: 1-20, 43, 45, or 47 may also include ADH polypeptides that are not shown in FIGS. 5 and 15. A person of ordinary skill in the art, however, could easily align these polypeptide sequences with the polypeptide sequences shown in FIGS. 5 and 15 to determine what additional variants could be made to the ADH polypeptides.

In another aspect of the present invention, constructs are provided. As used herein, the term “construct” refers to recombinant polynucleotides including, without limitation, DNA and RNA, which may be single-stranded or double-stranded and may represent the sense or the antisense strand. Recombinant polynucleotides are polynucleotides formed by laboratory methods that include polynucleotide sequences derived from at least two different natural sources or they may be synthetic. Constructs thus may include new modifications to endogenous genes introduced by, for example, genome editing technologies. Constructs may also include recombinant polynucleotides created using, for example, recombinant DNA methodologies.

The constructs provided herein may be prepared by methods available to those of skill in the art. Notably each of the constructs claimed are recombinant molecules and as such do not occur in nature. Generally, the nomenclature used herein and the laboratory procedures utilized in the present invention include molecular, biochemical, and recombinant DNA techniques that are well known and commonly employed in the art. Standard techniques available to those skilled in the art may be used for cloning, DNA and RNA isolation, amplification and purification. Such techniques are thoroughly explained in the literature.

The constructs provided herein may include a heterologous promoter operably linked to any one of the polynucleotides described herein. As used herein, the terms “heterologous promoter,” “promoter,” “promoter region,” or “promoter sequence” refer generally to transcriptional regulatory regions of a gene, which may be found at the 5′ or 3′ side of the ADH polynucleotides described herein, or within the coding region of the ADH polynucleotides, or within introns in the ADH polynucleotides. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

In some embodiments, the disclosed ADH polynucleotides are operably connected to the heterologous promoter. As used herein, a polynucleotide is “operably connected” or “operably linked” when it is placed into a functional relationship with a second polynucleotide sequence. For instance, a promoter is operably linked to an ADH polynucleotide if the promoter is connected to the ADH polynucleotide such that it may affect transcription of the ADH polynucleotides. In various embodiments, the ADH polynucleotides may be operably linked to at least 1, at least 2, at least 3, at least 4, at least 5, or at least 10 promoters.

Heterologous promoters useful in the practice of the present invention include, but are not limited to, constitutive, inducible, temporally-regulated, developmentally regulated, chemically regulated, tissue-preferred and tissue-specific promoters. The heterologous promoter may be a plant, animal, bacterial, fungal, or synthetic promoter. Suitable promoters for expression in plants include, without limitation, the 35S promoter of the cauliflower mosaic virus, ubiquitine, tCUP cryptic constitutive promoter, the Rsyn7 promoter, pathogen-inducible promoters, the maize In2-2 promoter, the tobacco PR-1a promoter, glucocorticoid-inducible promoters, estrogen-inducible promoters, tetracycline-inducible promoters, tetracycline-repressible promoters, and promoters for monocots like actin. Other promoters include the T3, T7 and SP6 promoter sequences, which are often used for in vitro transcription of RNA. In mammalian cells, typical promoters include, without limitation, promoters for Rous sarcoma virus (RSV), human immunodeficiency virus (HIV-1), cytomegalovirus (CMV), SV40 virus, and the like as well as the translational elongation factor EF-1α promoter or ubiquitin promoter. Those of skill in the art are familiar with a wide variety of additional promoters for use in various cell types. In some embodiments, the heterologous promoter includes a plant promoter, either endogenous to the plant host or heterologous.

Vectors including any of the constructs or polynucleotides described herein are provided. The term “vector” is intended to refer to a polynucleotide capable of transporting another polynucleotide to which it has been linked. In some embodiments, the vector may be a “plasmid,” which refers to a circular double-stranded DNA loop into which additional DNA segments may be ligated. Certain vectors are capable of autonomous replication in a host cell into which they are introduced (e.g., bacterial vectors having a bacterial origin of replication and episomal mammalian vectors). Other vectors can be integrated into the genome of a host cell upon introduction into the host cell, and thereby are replicated along with the host genome, such as some viral vectors or transposons. Plant mini-chromosomes are also included as vectors. Vectors may carry genetic elements, such as those that confer resistance to certain drugs or chemicals.

Cells including any of the polynucleotides, constructs, or vectors described herein are provided. Suitable “cells” that may be used in accordance with the present invention include eukaryotic or prokaryotic cells. Suitable eukaryotic cells include, without limitation, plant cells, fungal cells, and animal cells. Suitable prokaryotic cells include, without limitation, gram-negative and gram-positive bacterial species. In some embodiments, the cell is a plant cell such as, without limitation, a soybean plant cell, a mung bean plant cell, an opium poppy plant cell, a quinoa plant cell, an alfalfa plant cell, a rice plant cell, a wheat plant cell, a corn plant cell, a sorghum plant cell, a barley plant cell, a millet plant cell, an oat plant cell, a rye plant cell, a rapeseed plant cell, a beet plant cell, and a miscanthus plant cell. In some embodiments, the cell is a bacterial or fungal cell.

Plants including any of the polynucleotides, constructs, vectors, or cells described herein are also provided. Suitable plants may include, without limitation, a beet plant, a soybean plant, a mung bean plant, an opium poppy plant, a quinoa plant, an alfalfa plant, a rice plant, a wheat plant, a corn plant, a sorghum plant, a barley plant, a millet plant, an oat plant, a rye plant, and a rapeseed plant as well as perennial grasses such as a miscanthus plant. For example, ADH polynucleotides encoding any one of the ADH polypeptides of SEQ ID NOS: 1-20, 43, 45, or 47 may be used to generate transgenic plants.

Portions or parts of these plants are also useful and provided. Portions and parts of plants includes, without limitation, plant cells, plant tissue, plant progeny, plant asexual propagates, plant seeds. The plant may be grown from a seed comprising transgenic cells or may be grown by any other means available to those of skill in the art. Chimeric plants comprising transgenic cells are also provided and encompassed.

As used herein, a “plant” includes any portion of the plant including, without limitation, a whole plant, a portion of a plant such as a part of a root, leaf, stem, seed, pod, flower, cell, tissue plant germplasm, asexual propagate, or any progeny thereof. Germplasm refers to genetic material from an individual or group of individuals or a clone derived from a line, cultivar, variety or culture. Plant refers to whole plants or portions thereof including, without limitation, plant cells, plant protoplasts, plant tissue culture cells or calli. For example, a beet plant refers to whole beet plant or portions thereof including, without limitation, beet plant cells, beet plant protoplasts, beet plant tissue culture cells or calli. A plant cell refers to cells harvested or derived from any portion of the plant or plant tissue culture cells or calli.

Methods for increasing production of at least one product of the tyrosine or HPP pathways in a cell are provided. The methods may include introducing any of the polynucleotides, constructs, or vectors described herein into the cell. Suitable products of the tyrosine or HPP pathways include, without limitation, vitamin E, plastoquinone, a cyanogenic glycoside, a benzylisoquinoline alkaloid, rosmarinic acid, betalains, suberin, mescaline, morphine, salidroside, a phenylpropanoid compound, dhurrin, a tocochromanol, ubiquinone, lignin, a catecholamine such as epinephrine (adrenaline) or dopamine (i.e., L-dihydroxyphenylalanine (L-DOPA)), melanin, an isoquinoline alkaloid, hydroxycinnamic acid amide (HCAA), an amaryllidaceae alkaloid, hordenine, hydroxycinnamate, hydroxylstyrene, or tyrosine. Phenylpropanoid compounds (i.e., lignin, tannins, flavonoids, stilbene) may be produced from tyrosine, for example, by combining the polypeptides disclosed herein with a tyrosine-ammonia lyase (TAL) or by using cells that naturally have a TAL such as grass cells.

As used herein, “introducing” describes a process by which exogenous polynucleotides (e.g., DNA or RNA) are introduced into a recipient cell. Methods of introducing polynucleotides into a cell are known in the art and may include, without limitation, microinjection, transformation, and transfection methods. Transformation or transfection may occur under natural or artificial conditions according to various methods well known in the art, and may rely on any known method for the insertion of foreign nucleic acid sequences into a host cell. The method for transformation or transfection is selected based on the type of host cell being transformed and may include, but is not limited to, the floral dip method, Agrobacterium-mediated transformation, bacteriophage or viral infection, electroporation, heat shock, lipofection, and particle bombardment. Microinjection of polynucleotides may also be used to introduce polynucleotides into cells.

In some embodiments, the present methods may further include purifying the product of the tyrosine or HPP pathways from the cells. As used herein, the term “purifying” is used to refer to the process of ensuring that the product of the tyrosine or HPP pathways is substantially or essentially free from cellular components and other impurities. Purification of products of the tyrosine or HPP pathways is typically performed using analytical chemistry techniques such as high performance liquid chromatography (HPLC) and other chromatographic techniques. Methods of purifying such products are well known to those skilled in the art. A “purified” product of the tyrosine or HPP pathways means that the product is at least 85% pure, more preferably at least 95% pure, and most preferably at least 99% pure.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference in their entirety, unless explicitly indicated otherwise. The present disclosure shall control in the event there are any disparities between any definitions and/or description found in the cited references.

Unless otherwise specified or indicated by context, the terms “a”, “an”, and “the” mean “one or more.” For example, “a protein” or “an RNA” should be interpreted to mean “one or more proteins” or “one or more RNAs,” respectively.

The following examples are meant only to be illustrative and are not meant as limitations on the scope of the invention or of the appended claims.

### EXAMPLES

### Example 1—Relaxation of Tyrosine Pathway Regulation Underlies the Evolution of Betalain Pigmentation in Caryophyllales

This Example is based on data reported in Lopez-Nieves et al., “Relaxation of Tyrosine Pathway Regulation Underlies the Evolution of Betalain Pigmentation in Caryophyllales,” New Phytologist, 217(2):896-908 (2018), the contents of which (including all supplemental data, figures, and associated materials) is incorporated herein by reference.

### Summary

Diverse natural products are synthesized in plants by specialized metabolic enzymes, which are often lineage-specific and derived from gene duplication followed by functional divergence. However, little is known about the contribution of primary metabolism to the evolution of specialized metabolic pathways.
Betalain pigments, uniquely found in the plant order Caryophyllales, are synthesized from the aromatic amino acid L-tyrosine (Tyr) and replaced the otherwise ubiquitous phenylalanine-derived anthocyanins. This study combined biochemical, molecular and phylogenetic analyses and uncovered coordinated evolution of Tyr and betalain biosynthetic pathways in Caryophyllales.
We found that Beta vulgaris, which produces high levels of betalains, synthesizes Tyr via plastidic arogenate dehydrogenases (TyrAₐ/ADH) encoded by two ADH genes (BvADHα and BvADHβ). Unlike BvADHβ and other plant ADHs that are strongly inhibited by Tyr, BvADHα exhibited relaxed sensitivity to Tyr. Also, Tyr-insensitive BvADHα orthologs arose during the evolution of betalain pigmentation in the core Caryophyllales and later experienced relaxed selection and gene loss in lineages that reverted from betalain to anthocyanin pigmentation, such as Caryophyllaceae.
These results suggest that relaxation of Tyr pathway regulation increased Tyr production and contributed to the evolution of betalain pigmentation, highlighting the significance of upstream primary metabolic regulation for the diversification of specialized plant metabolism.

### Introduction

Plants synthesize numerous specialized metabolites (also known as secondary metabolites), which play crucial roles in plant adaptation. In contrast to well-documented diversification of plant enzymes directly involved in specialized metabolism (Chen et al., 2011; Mizutani & Ohta, 2010; Moghe & Last, 2015; Pichersky & Lewinsohn, 2011; Weng, 2014), relatively little is known about the evolution of primary metabolic enzymes that provide precursors to the production of various specialized metabolites.

L-Tyrosine (Tyr) is an essential aromatic amino acid required for protein biosynthesis in all organisms; however, it is synthesized de novo only in bacteria, fungi, and plants, but not in animals. Consequently, animals have to consume Tyr or L-phenylalanine (Phe) that can be hydroxylated to Tyr (Pencharz et al., 2007). Besides protein biosynthesis, plants also use Tyr to produce a diverse array of specialized metabolites that are important for defense (e.g. dhurrin, Gleadow & Møller, 2014), stress tolerance (e.g. tocopherols, Mene-Saffrane et al., 2010), and pollinator attraction (e.g., betalains, Tanaka et al., 2008). Notably, humans have a long history of utilizing Tyr-derived specialized metabolites, such as the psychedelic alkaloid mescaline derived from the cactus Lophophora williamsii (Ibarra-Laclette et al., 2015) and the analgesic morphine derived from Papaver somniferum (opium poppy, Beaudoin & Facchini, 2014; Millgate et al., 2004).

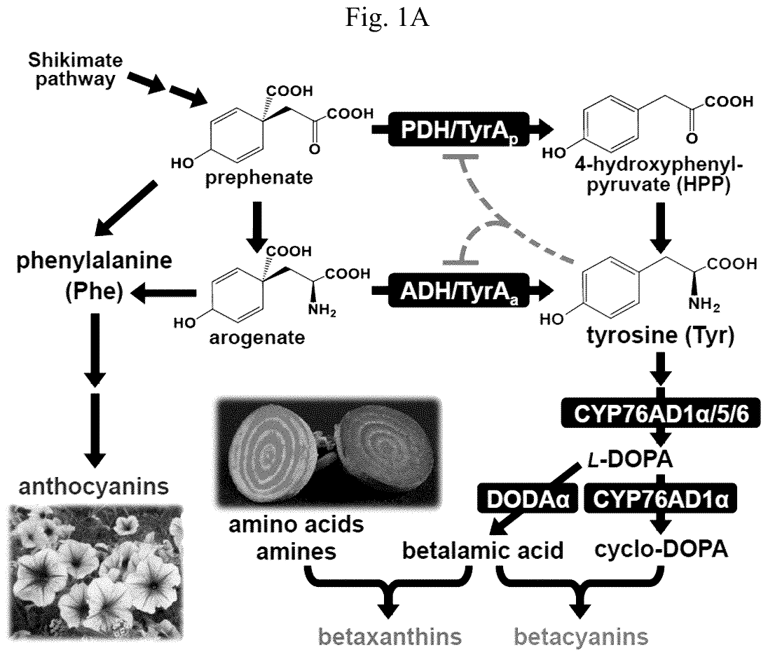

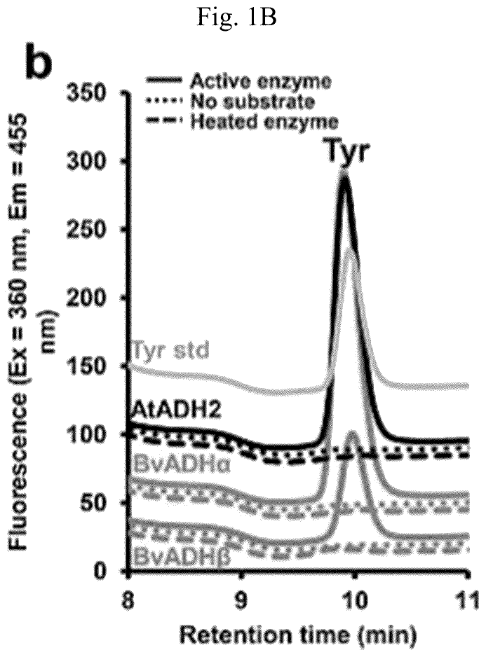

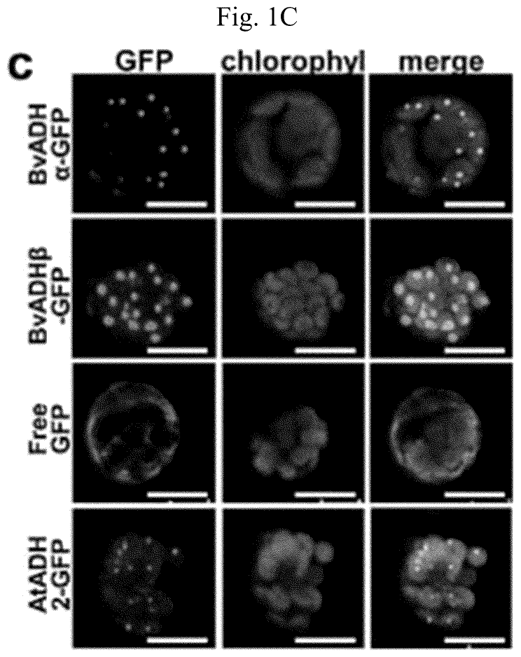

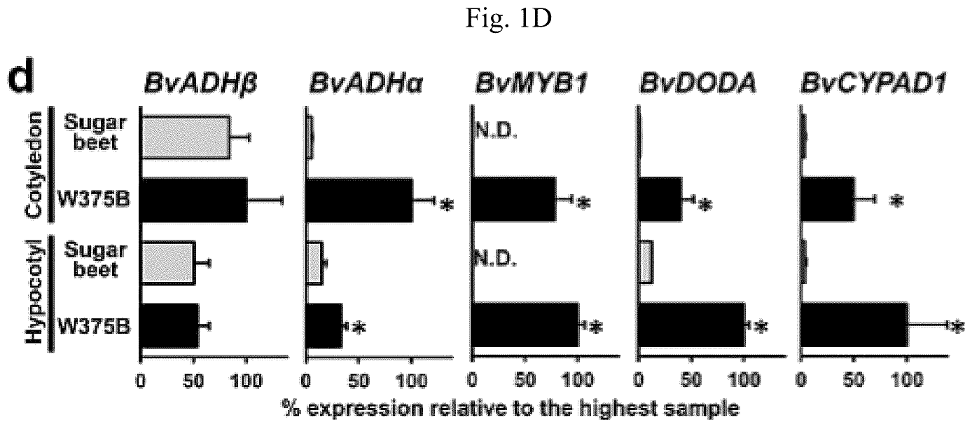

Tyr is synthesized from prephenate, which is converted from the final product of the shikimate pathway, chorismate (Maeda & Dudareva, 2012; Siehl, 1999; Tzin, V. & Galili, 2010). In most bacteria and fungi, prephenate is oxidatively decarboxylated by prephenate dehydrogenase (TyrAₚ/PDH, hereafter referred only as PDH; EC 1.3.1.12) to 4-hydroxyphenylpyruvate (HPP), which is transaminated to Tyr (Bentley, 1990, FIG. 1A). On the other hand, most plants first transaminate prephenate into arogenate and subsequently decarboxylate into Tyr by arogenate dehydrogenase (TyrAₐ/ADH, hereafter referred only as ADH; EC 1.3.1.78, Rippert & Matringe, 2002a,b), both steps occurring in the plastids (Dal Cin et al., 2011; Rippert et al., 2009; FIG. 1A). The Tyr pathway is usually highly regulated at PDH and ADH. These homologous enzymes are strongly feedback inhibited by Tyr and control carbon flow between the two competing Tyr and Phe pathways (Gaines et al., 1982; Bentley, 1990; Rippert & Matringe, 2002a,b; FIG. 1B). A recent report showed that, in addition to plastidic ADH enzymes, some plants possess a PDH enzyme(s) that is not inhibited by Tyr and is localized to the cytosol (Rubin & Jensen, 1979; Schenck et al., 2015; 2017; Siehl, 1999). Clearly, there is evolutionary variation in the Tyr pathway(s) in different plant lineages that warrants investigation.

Betalains are a class of Tyr-derived pigments that, within the flowering plants, occur exclusively in the order Caryophyllales where they replace the otherwise ubiquitous anthocyanins (Mabry, 1964; Tanaka et al., 2008). Within Caryophyllales, the majority of families are betalain pigmented. In two families, Molluginaceae and Caryophyllaceae, however, evolutionary reversions from betalain to anthocyanin pigmentation have occurred (Brockington et al., 2015), highlighting the fact that these two classes of water-soluble pigments have never been found in the same organism (Bate-Smith, 1962; Brockington et al., 2011; Clement & Mabry, 1996; Mabry, 1964). Betalains and anthocyanins are synthesized from Tyr and Phe, respectively, but have similar physiological functions in pollinator attraction and stress tolerance (Tanaka et al., 2008). Betalains are also used as a natural food dye (E162) and have anticancer and antidiabetic properties (Khan, 2015; Lee et al., 2014; Neelwarne & Halagur, 2012). Furthermore, intermediates in the betalain pathway are important pharmaceuticals [e.g. L-dihydroxyphenylalanine (L-DOPA) for the treatment of Parkinson's disease] or are substrates for other pharmaceutical agents (e.g. the production of dopamine and isoquinoline alkaloids such as morphine). Consequently, understanding the coordinated regulation of Tyr and betalain biosynthesis has the potential to enhance the production of Tyr, and the yield of Tyr-derived plant natural products important for human health and nutrition.

Betalain biosynthesis starts with hydroxylation of Tyr to L-DOPA by at least three closely related cytochrome P450 enzymes (CYP76AD1, CYP76AD5, and CYP76AD6, FIG. 1A) (Polturak et al., 2016; Sunnadeniya et al., 2016). L-DOPA is further converted into betalamic acid or cyclo-DOPA by L-DOPA dioxygenases (DODA, Christinet et al., 2004; Gandia-Herrero & Garcia-Carmona, 2012) or CYP76AD1 (Hatlestad et al., 2012), respectively (FIG. 1A). Betalamic acid then spontaneously reacts with cyclo-DOPA or amines to produce various forms of betacyanins or betaxanthins, respectively, which are usually further glycosylated. Recent studies found that the two key enzymes within the betalain pathway, DODA, and CYP76AD1, duplicated just prior to the emergence of betalain pigmentation (Brockington et al., 2015). Subsequently, one of the duplicated copies (DODAa and CYP76AD1α) in both genes became specialized for betalain biosynthesis and were lost or downregulated in the anthocyanin-producing families such as Molluginaceae and Caryophyllaceae (Brockington et al., 2015). Despite recent and rapid progress in understanding the betalain pathway enzymes and their evolution, little is known about the regulation of primary Tyr metabolism in relation to the evolution of this novel Tyr-dependent betalain pathway.

Here we first investigated the Tyr biosynthetic pathway and its regulation in table beet (Beta vulgaris L.), which produces high levels of betalains (Goldman, 1996). Using comparative genomics, biochemical, and cellular analyses, we found plastidic ADH enzymes from B. vulgaris that exhibit relaxed sensitivity to Tyr inhibition in vitro and in vivo. Phylogenetic analysis combined with recombinant enzyme characterization further demonstrated that de-regulated ADH enzymes emerged during the evolution of betalain pigmentations in the core Caryophyllales, and were lost or downregulated following disappearance of betalains. Furthermore, transient expression of the de-regulated ADH in Nicotiana benthamiana led to high accumulation of Tyr in planta. The results revealed the important contribution of primary Tyr pathway regulation to the unique evolution of a plant specialized metabolic pathway, betalain biosynthesis.

### Materials and Methods

Plant Source and Growth Conditions B. vulgaris varieties, red beet (W357B), yellow beet (Touch Stone), and white beet (Blankoma), were provided by Dr. Irwin Goldman from the University of Wisconsin-Madison, Department of Horticulture (Goldman, 1996), whereas sugar beet (Big Buck) and sea beet (PI 562585) were commercial sugar beets obtained from the Heirloom Seeds (West Finley, PA, USA) and the National Plant Germplasm System (NPGS), respectively. Spinach (Spinacia oleraceae), Pigeonberry (Rivina humilis), four o'clock (Mirabilis jalapa), and common purslane (Portulaca oleracea) were grown from seed with a growing mix soil (Fafard®, Agawam, MA, USA) in a growth chamber under 12 hr light (100 μE), 22° C. and 60% humidity. After one month of growth, their leaves were harvested for RNA extraction.

Identification and Cloning of ADH Homologs from Caryophyllales

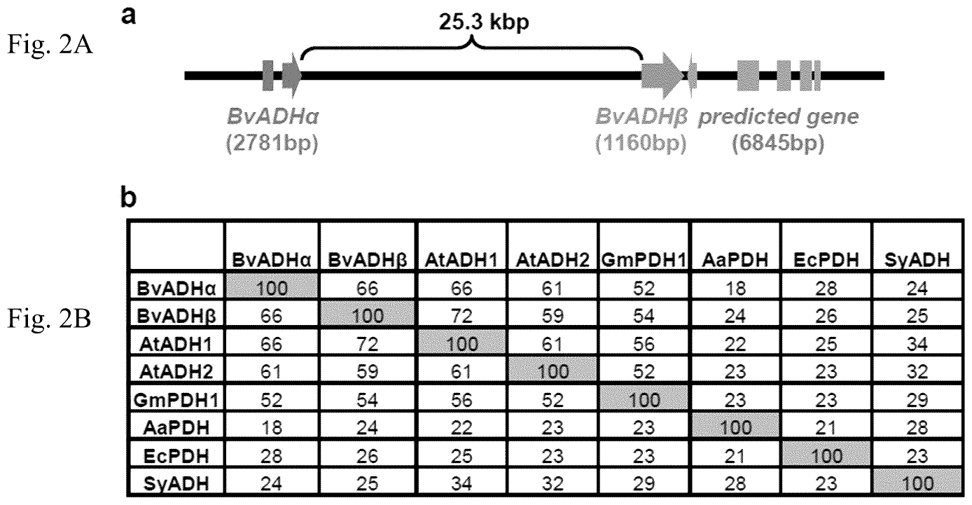

BLASTP searches were performed using the protein sequences of ADH and PDH enzymes from A. thaliana (AtADH1/At5g34930, NP_173023; AtADH2/At1g15710, NP_198343), Glycine max (GmPDH, KM507071), Synechocystis sp. PCC6803 (SyADH, WP_010872597), Escherichia coli (EcPDH, WP_052912694), Aquifex aeolicus (AaPDH, WP_010881139) as queries against the sugar beet genome (Beta vulgaris molgen.mpg.de/) (FIG. 2B). Potential ADH candidates were identified based on a broad phylogenetic analysis that included various plant ADH and PDH sequences.

Genomic DNA was extracted using Tris-sodium chloride-EDTA/sodium dodecyl sulfate buffer and precipitated with isopropanol and 200 mM ammonium acetate. For RNA isolation, the method described by Wang et al (2011) was used with some modifications. The tissues were ground in a mortar with liquid nitrogen and powder polyvinylpyrrolidone (PVP). After addition of 700 μL fresh pre-warmed lysis buffer (2% CTAB, 2 M NaCl, 100 mM Tris-HCl pH 8, 25 mM EDTA and 5% P-mercaptoethanol), the samples were shaken vigorously for 2 min and incubated in a water bath at 65° C. for 5 min. The RNA was converted into complementary DNA (cDNA) using the High-Capacity cDNA Reverse Transcription Kit (Applied Biotechnology, USA) and SuperScript IV Reverse Transcriptase with oligo dT₂₀ primer or random primers (Invitrogen, USA).

Cloning primers were designed with the Invitrogen primer design (lifetechnologies.com) and the PCR In-Fusion® primers designing program (clontech.com, Clontech, Mount View, CA). All ADH candidate genes, except for PoADHα (see below), were PCR amplified from cDNA using gene-specific primers (Table 1) and Phusion DNA polymerase (Thermo, Waltham, MA) with the following conditions: initial denaturation at 95° C. for 5 min, 35 cycles of amplification at 95° C. for 30 s, 58° C. for 30 s, 72° C. for 30 s, with a final extension at 72° C. for 10 min. The PCR fragments were purified using QIAquick gel extraction kit (Qiagen, Valencia, CA) and were inserted into the pGEX-2T vector (GE Healthcare) at EcoRI and BamHI sites using the In-Fusion cloning method (Clontech). PoADHα was gene synthesized (Biomatik, Cambridge, Ontario, Canada) and directly cloned into the same pGEX-2T vector. For generation of His-tagged proteins, the cloned PCR fragments were inserted into the pET28a vector (Novagen, Madison WI, USA) at NdeI and EcoRI site.

TABLE 1
Primers used as indicated in the description and methods

| Species (gene) | Purpose | Primer name | Primer sequence 5′ to 3′ |
|---|---|---|---|
| Beta vulgaris | RT- | pHM0290SLN | GGTTCCGCGTGGATCCCTAACAATTC |
| (BvADHβ) | PCR | BvADHβF | GCAGCAT (SEQ ID NO: 49) |
| Beta vulgaris | RT- | pHM0291SLN | AATTCGGAGACAAATTGAGAATTCA |
| (BvADHβ) | PCR | RBvADHβR | TCGTGACTG (SEQ ID NO: 50) |
| Beta vulgaris | RT- | pHM0372SLN | CTGGTTCCGCGTGGATCCTGCGGTGG |
| (BvADHα) | PCR | BvADHαF | AGGTGGTTCG (SEQ ID NO: 51) |
| Beta vulgaris | RT- | pHM0373SLN | GTTAATGGTACTAGATAGGAATTCAT |
| (BvADHα) | PCR | BvADHαR | CGTGACTGA (SEQ ID NO: 52) |
| Arabidopsis thaliana | Cloning | pHM0384SLN | CTGGTTCCGCGTGGATCCGCAATCG |
| (AtADH2) | | AtADHαF | ACGCCGCCCAA (SEQ ID NO: 53) |
| Arabidopsis thaliana | Cloning | pHM0385SLN | TCATCATCATCATCTTAAGAATTCAT |
| (AtADH2) | | AtADHαR | CGTGACTGA (SEQ ID NO: 54) |
| Spinacia oleracea | Cloning | pHM0582SoA | CTGGTTCCGCGTGGATCCGCCGCTAC |
| (SoADHβ) | | DHβF | CAATACCTCC (SEQ ID NO: 55) |
| Spinacia oleracea | Cloning | pHM0583SoA | AATTCAGAGATCAATTGAGAATTCA |
| (SoADHβ) | | DHβR | TCGTGACTGA (SEQ ID NO: 56) |
| Spinacia oleracea | Cloning | pHM0584SoA | CTGGTTCCGCGTGGATCCTGCGCCGC |
| (SoADHα) | | DHαF | CTCTGACTCC (SEQ ID NO: 57) |
| Spinacia oleracea | Cloning | pHM0585SoA | TGGTAATAATTCTAGATAGGAATTCA |
| (SoADHα) | | DHαR | TCGTGACTGA (SEQ ID NO: 58) |
| Nepenthes alata | Cloning | pHM0603SLN | CTGGTTCCGCGTGGATCCGCCGCGCT |
| (NaADHβ) | | NaADHF | GCCAAACGACT (SEQ ID NO: 59) |
| Nepenthes alata | Cloning | pHM0604SLN | AAATGTTGAGAGAAATTGAGAATTC |
| (NaADHβ) | | NaADHR | ATCGTGACTGA (SEQ ID NO: 60) |
| Portulaca | RT- | pHM0609SLN | CTGGTTCCGCGTGGATCCTGCTCATC |
| oleracea(PoADHα) | PCR | PoADHαAF | ATCATCATCAT (SEQ ID NO: 61) |
| Portulaca | RT- | pHM0610SLN | CGTCAACGATAGATCATAGGAATTC |
| oleracea(PoADHα) | PCR | PoADHαAR | ATCGTGACTGA (SEQ ID NO: 62) |
| Mirabilis | Cloning | pHM0624SLN | CTGGTTCCGCGTGGATCCATAGCGAT |
| jalapa(MjADHα) | | MjADHαAF | AGTTGGGTTTG (SEQ ID NO: 63) |
| Mirabilis | Cloning | pHM0625SLN | TATCAATGGTCGTCGATAGGAATTCA |
| jalapa(MjADHα) | | MjADHαAR | TCGTGACTGA (SEQ ID NO: 64) |
| Rivina | Cloning | pHM0647SLN | CTGGTTCCGCGTGGATCCTGCACGGC |
| humilis(RhADHα) | | RhADHαF | CTTCACTAAAAC (SEQ ID NO: 65) |
| Rivina | Cloning | pHM0648SLN | TCAATGGATCAAAGCGGTAGGAATT |
| humilis(RhADHα) | | RhADHαR | CATCGTGACTGA (SEQ ID NO: 66) |
| Beta vulgaris | RT- | BvADHα_q_F | TCAAGCTGAGGTTACTTTTGACA |
| (BvADHα) | PCR | | (SEQ ID NO: 67) |
| Beta vulgaris | RT- | BvADHα_q_R | AAGAAGCATGATTTAGTGGTGGT(SE |
| (BvADHα) | PCR | | Q ID NO: 68) |
| Beta vulgaris | RT- | BvADHβ_q_F | TGCAGCGACTTAAACGATCG (SEQ |
| (BvADHβ) | PCR | | ID NO: 69) |
| Beta vulgaris | RT- | BvADHβ_q_R | TTGGGGAAGTTTGCCGTTTG(SEQ ID |
| (BvADHβ) | PCR | | NO: 70) |
| Beta vulgaris | RT- | pHM0793SLN | AGTTCCCTCTGCTGATATG (SEQ ID |
| (BvADHα) | PCR | BvADHαF | NO: 71) |
| Beta vulgaris | RT- | pHM0794SLN | GTGGTTAATGGTACTAGATAG (SEQ |
| (BvADHα) | PCR | BvADHαR | ID NO: 72) |
| Beta vulgaris | qPCR | pHM0791SLN | GCGAAGGAGATCAAATTTCT (SEQ ID |
| (BvADHβ) | | BvADHβF | NO: 73) |
| Beta vulgaris | qPCR | pHM0792SLN | TCAATTTGTCTCCGAATTTGC (SEQ |
| (BvADHβ) | | BvADHβR | ID NO: 74) |
| Beta vulgaris | qPCR | BvADHα F | ATGATTTCACTCTCTTCTTTTCATCC |
| (BvADHα) | | | (SEQ ID NO: 75) |
| Beta vulgaris | qPCR | BvADHα R | GATTTAGTGGTGGTTAATGGTACTAG |
| (BvADHα) | | | ATAG (SEQ ID NO: 76) |
| Beta vulgaris | qPCR | BvADHβ F | ATGCTTTCTCTCTCCTCCAC (SEQ ID |
| (BvADHβ) | | | NO: 77) |
| Beta vulgaris | qPCR | BvADHβ R | CAAATTCGGAGACAAATTGA (SEQ |
| (BvADHβ) | | | ID NO: 78) |
| Beta vulgaris | qPCR | pHM0001HM | TCTATCCTTGCATCTCTCAG (SEQ ID |
| (BvActin) | | BvACT | NO: 79) |
| Beta vulgaris | qPCR | pHM0002HM | TCTCCAAGGGCGAGTATGAT (SEQ ID |
| (BvActin) | | BvACT | NO: 80) |
| Beta vulgaris | qPCR | pHM0003HM | CATTGGTTCAGGAAGTGCAA (SEQ ID |
| (BvDODA) | | BvDODA | NO: 81) |
| Beta vulgaris | qPCR | pHM0004HM | CCTTTGATTCATGGCTTCGT (SEQ ID |
| (BvDODA) | | BvDODA | NO: 82) |
| Beta vulgaris | qPCR | pHM0576Bv | TATCAAACGAGGGCACTTC (SEQ ID |
| (BvMYB1) | | MYB1F | NO: 83) |
| Beta vulgaris | qPCR | pHM0577Bv | GATGGTCTTTGATAGCAGC (SEQ ID |
| (BvMYB1) | | MYB1R | NO: 84) |
| Beta vulgaris | qPCR | pHM0005HM | CTTTTCAGTGGAATTAGCCCACC |
| (BvCYP76AD1) | | BvCYP76AD1 | (SEQ ID NO: 85) |
| Beta vulgaris | qPCR | pHM0006HM | TGGAACATTATGGAAGATATTGGG |
| (BvCYP76AD1) | | BvCYP76AD1 | (SEQ ID NO: 86) |
| GFP | qPCR | tGFP_q_F | GGCTGGAAGAGTGATCGGAG (SEQ |
| | | | ID NO: 87) |
| GFP | qPCR | tGFP_q_R | ACGCTACTGTTGAGCATCTTCA (SEQ |
| | | | ID NO: 88) |
| Gene Racer oligoT | RT- | GeneRacer | GCTGTCAACGATACGCTACGTAACG |
| | PCR | OligoT | GCATGACAGTG(T)20 (SEQ ID NO: 89) |
| Eukaryotic | qPCR | EF1α_q_F | AGCTTTACCTCCCAAGTCATC (SEQ |
| translational | | | ID NO: 90) |
| elongation factor 1α | | | |
| Eukaryotic | qPCR | EF1α_q_R | CCAAGATTGACAGGCGTTCT (SEQ ID |
| translational | | | NO: 91) |
| elongation factor 1α | | | |

### Recombinant Enzyme Expression and Purification

The His-tagged recombinant protein expression was carried out as we described previously (Dornfeld et al., 2014). For GST-tagged recombinant protein expression, the cloned pGEX-2T vectors were introduced into Rosetta-2 E. coli competent cells (Novagen, Madison WI, USA) and cultured overnight at 37° C., 200 r.p.m. in 10 mL LB medium containing Ampicillin (100 μg/mL). The ten milliliters of the overnight culture were transferred to 1 μL LB medium with Ampicillin (100 μg/mL and further incubated at 37° C. and 200 r.p.m. until the OD₆₀₀ reached 0.3. The temperature was then changed to 18° C. and, after 1 hr, isopropyl β-D-1-thiogalactopyranoside (IPTG, 400 mM final concentration) was added to induce recombinant protein expression. After overnight incubation at 18° C. under constant shaking at 200 r.p.m., cultures were harvested by centrifugation at 2,000 g for 10 min at 4° C., and the pellet was washed with 0.9% NaCl solution. The samples were harvested and resuspended in 25 mL of lysis buffer [phosphate-buffered saline (PBS) pH 7.4, 1 mM phenylmethylsulfonyl fluoride (PMSF), 1 mM dithiothreitol (DTT) and plant proteases inhibitor cocktail (Amresco, Solon, OH, USA)]. The resuspended cells were sonicated for periods of 20 s for 5 min. The cell lysate was centrifuged at 10,000 g for 30 min at 4° C., and the supernatant was applied to Fast Protein Liquid Chromatography (FPLC, AKTApure25 FPLC system, GE Healthcare) equipped with GSTtrap™ FF (GE Healthcare, USA). Prior and after injection, the column was washed with five times bed volume wash buffer A (PBS, pH 7.6) followed by five times bed volume of wash buffer B (10 mM glutathione, 1.54 g of reduced glutathione dissolved in 500 mL of 50 mM Tris-HCl, pH 8). The recombinant enzymes containing GST-tag were eluted with ten-bed volumes of the elution buffer B and collected into Eppendorf tubes containing 500 L. Recombinant enzymes eluted in the fraction five and six, which were combined and desalted using a gel filtration column (Sephadex G50-80 resin, Sigma-Aldrich, St Louis, MO, USA) in the reaction buffer [200 mM HEPES (pH 7.6), 50 mM KCl, 10% ethylene glycol]. Enzyme concentrations were measured using Bradford assay (Bio-Rad, Des Plaines, IL, USA) and the enzyme purity was estimated by running on SDS-PAGE gel and analyzing with ImageJ (imagej.nih.gov).

### ADH and PDH Activity Assays In Vitro

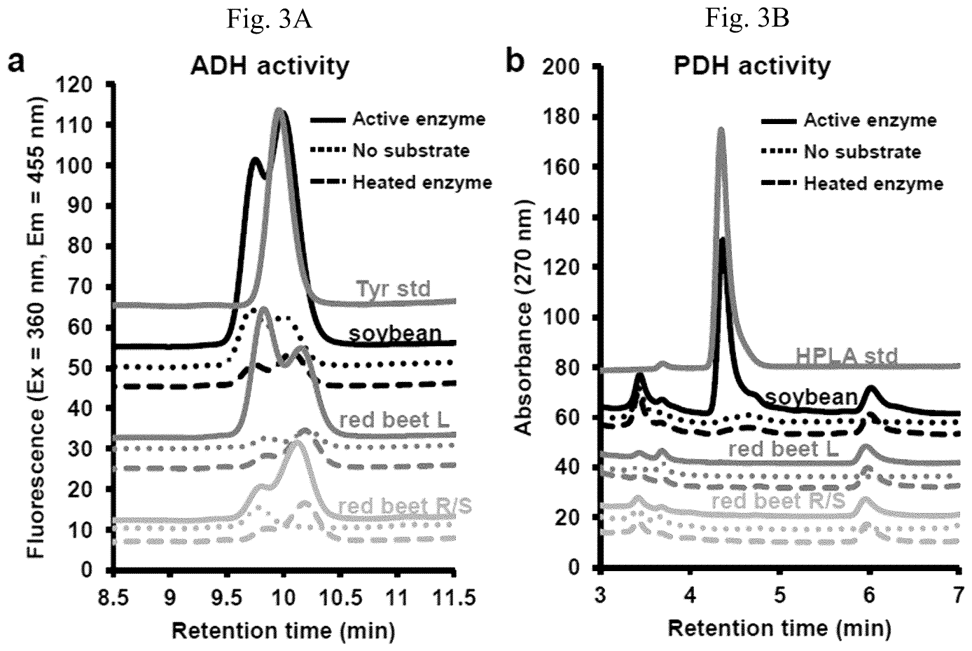

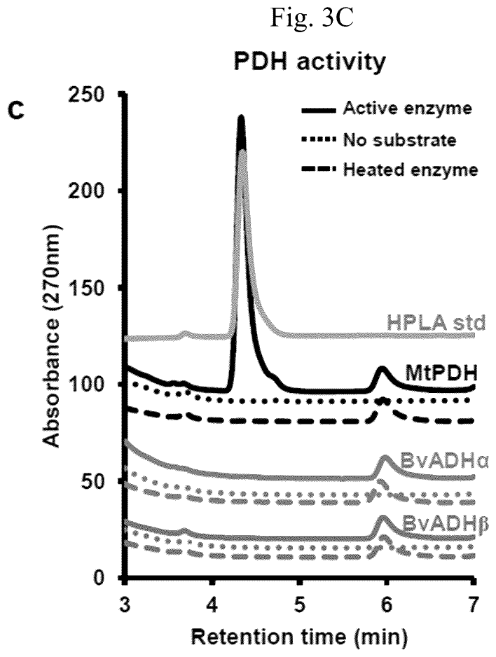

ADH and PDH activity from beet tissues (FIGS. 3A, 3B) were analyzed by using the leaves and stem/root crude protein extract of red beet (W357B). The beets were grown in a greenhouse for 12 weeks with a temperature of 22-25° C. and 16 hr of ambient and supplemented lights. Protein extraction was performed by grinding 1 g of tissues in liquid nitrogen and resuspending the powder in the extraction buffer [200 mM HEPES (pH 7.6), 50 mM KCl, 10% ethylene glycol, 1 mM PMSF, 1 mM DTT and plant proteases inhibitor cocktail (Ameresco)]. The extracts were desalted using the gel filtration column (Sephadex G50-80 resin, Sigma-Aldrich St. Louis, MO, USA) into the reaction buffer. The ADH or PDH assays were performed by mixing the desalted protein extract with 1 mM NADP⁺ and 1 mM L-arogenate or prephenate in a total volume of 10 μL or 25 L, respectively. L-Arogenate was prepared by enzymatic conversion from prephenate (Sigma-Aldrich, St. Louis, MO, USA), as previously described (Schenck et al., 2015). The reactions were started by adding the enzyme (crude extract or recombinant enzyme) and incubated at 37° C. for 45 min. The reaction was stopped with two times volume of methanol. The same ADH and PDH assay protocols were used for initial characterization of purified recombinant BvADH enzymes

For detection of Tyr product from the ADH assays, 10 μL of the reaction mixture was first derivatized with the equal volume of the 40.26 mM OPA solution [5.4 mg OPA (Sigma-Aldrich, St. Louis, MO, USA) mixed in 100 μL methanol, 5 μL 2-mercaptoethanol and 900 L 0.4M boric acid) for 3 min, injected to high pressure liquid chromatography (HPLC, Agilent 1260) equipped with the Eclipse XDH-C18 column (5 m, 3.0×150 mm, Agilent, USA), and separated by a 30 min linear gradient from 20-45% methanol in 0.1% ammonium acetate at a flow rate of 0.8 ml/min. The substrate and product of ADH assays (Tyr and arogenate, respectively) were detected by a fluorescence detector (Agilent, USA) with excitation at 360 nm and emission of 455 nm. For PDH assays, the reactions were stopped by addition of NaBH₄, which converts the reaction product HPP into hydroxyphenyllactic acid (HPLA), followed by neutralization with 100 μl of 6 N HCl as described by Schenck et al. 2015. The HPLC was equipped with ZORBAX SB-C18 column (Agilent, USA) using a 6 min isocratic elution at 25% methanol in 0.1% phosphoric acid, followed by a 20 min linear gradient of 25-60% methanol at a flow rate of 1.0 mL/min. The HPLA were monitored by absorption at 270 nm.

To test the electron donor and substrate preferences of purified recombinant enzymes, the ADH and PDH reactions were performed as described above, except for 12 min with 400 μM L-arogenate and 1 mM cofactor (NAD⁺ or NADP⁺). The reaction was stopped by placing the tubes on ice and immediately measured for the production of the reduced cofactor, NAD(P)H, at 340 nm by spectrophotometer (NanoDrop 2000, Thermo Scientific, USA). The quantification was based on the standard curve of authentic NADPH.

To examine Tyr sensitivity of the purified recombinant enzymes, ADH assay was performed as described previously (Schenck et al., 2015) but in the presence or absence of different concentrations of L-Tyr. Tyr was first dissolved in 0.025 N NaOH at 100 mM (as the water solubility of Tyr is very low, <2 mM), which was diluted to 4 mM to 10 μM final concentration in 0.0025 N NaOH. The reactions contained 500 mM HEPES (pH 7.6) to maintain the final pH at 7.6. The production of reduced cofactor (NADPH) was monitored at 340 nm using a spectrophotometer every two minutes for 10 min. In addition, other effectors (L-Phe, L-Trp, and betanin) were used to test possible inhibition of the enzyme ADH activity at a final concentration of 1 mM. All of the reactions were performed under non-saturated condition, where activity increased linearly depending on reaction times and enzyme concentrations.

Transient Expression of BvADHα and BvADHβ in Nicotiana benthamiana

ADHα and ADHβ sequences used for N. benthamiana agroinfiltration were amplified from Beta vulgaris var. vulgaris variety “Boltardy” (Chiltern Seeds, UK) swollen hypocotyl and leaf tissue cDNA libraries respectively, which were prepared using BioScript Reverse Transcriptase (Bioline Reagents, London, UK). Transcripts were amplified by PCR using gene specific primers (Table S1) and Phusion High-Fidelity DNA polymerase (Thermo Fisher Scientific, Waltham, MA, USA). Vectors for transient transformation were constructed with Golden Gate cloning using the MoClo Tool Kit (Weber et al., 2011; Addgene, Cambridge, MA, USA), with the BpiI and BsaI restriction sites eliminated after cloning. The turboGFP sequence used in this assay was a variant codon-optimized for plants contained in the MoClo Plant Parts Kit (Engler et al., 2014; Addgene, Cambridge, MA, USA). BvADHα, BvADHβ, and turboGFP sequences were ultimately cloned into the pICH86988 binary vector under control of the Cauliflower Mosaic Virus 35S promoter and the Agrobacterium tumefaciens octopine synthase (OCS) terminator.

Transient gene expression assays in N. benthamiana were performed according to the previously described agroinfiltration method with some modifications (Sparkes et al., 2006). All constructs were transformed into the Agrobacterium tumefaciens GV3101 strain, and grown in LB media supplemented with kanamycin (50 mg/L), gentamycin (25 mg/L) and rifampicin (50 mg/L) until reaching an OD₆₀₀ of 1.5. Cultures were then brought to a final OD₆₀₀ of 0.5 in infiltration media (10 mM MgCl2, 0.1 mM acetosyringone, 10 mM MES at pH 5.6) for three hours prior to infiltration. Infiltration spots corresponding to 35S::BvADHα, 35S::BvADHβ, and 35S::turboGFP were performed in the same leaves of 6-week old N. benthamiana plants alternating the position of the spots between plants in a clockwise manner to account for intra-leaf variation (Barshandy et al., 2015). Infiltrated tissue was sampled three days post-infiltration from five biological replicates for tyrosine quantification and qRT-PCR analysis.

For quantification of tyrosine and other amino acids, ˜40 mg fresh weight tissues were harvested, lyophilized, sent from the University of Cambridge (UK) to the University of Wisconsin-Madison (USA), and analyzed exactly as described. Tyrosine and other amino acids were extracted and measured as described previously (Wang et al., 2017). Amino acid standards (Sigma-Aldrich, St. Louis, MO, USA) of 4 to 1000 μM were prepared the same way to make standard curves.

### Phylogenetic Analysis

Amino acid sequences from genomes (full open reading frame) and transcriptomes (full or partial open reading frame) of Brockington et al. (2015) were used for phylogenetic analysis following methods described in Brockington et al. (2015) with minor modifications. In addition, we carried out analysis of dN/dS ratio in ADHα to test for relaxed selection in anthocyanic lineages (Table 2).

TABLE 2
Sequences of Caryophyllales (ingroups) and non-
Caryophyllales (outgroups) used in this Example.

| | | | |
|---|---|---|---|
| Taxon | Source | Accession code | Citation |

Ingroups

| | | | |
|---|---|---|---|
| [symbol] _ [symbol] _ [symbol] | Smith Lab | MJM1677 | (Brockington et al., 2015) |
| [symbol] _ [symbol] _ [symbol] | [symbol] | GJNX | (Matasci et al., 2014) |
| [symbol] _ [symbol] _ [symbol] | [symbol] | BJKT | (Matasci et al., 2014) |
| [symbol] _ [symbol] _ [symbol] | [symbol] | HZTS | (Matasci et al., 2014) |
| [symbol] _ [symbol] _ [symbol] | [symbol] | EDIT | (Matasci et al., 2014) |
| [symbol] _ [symbol] _ [symbol] | [symbol] | OMYK | (Matasci et al., 2014) |
| [symbol] _ [symbol] _ [symbol] | [symbol] | BERS | (Matasci et al., 2014) |
| [symbol] _ [symbol] _ [symbol] | [symbol] | HDSY | (Matasci et al., 2014) |
| [symbol] _ [symbol] _ [symbol] | [symbol] | PDQH | (Matasci et al., 2014) |
| [symbol] _ [symbol] _ [symbol] | [symbol] | ZBPY | (Matasci et al., 2014) |
| [symbol] _ [symbol] _ [symbol] | [symbol] | OHKC | (Matasci et al., 2014) |
| [symbol] _ [symbol] _ [symbol] | [symbol] | BWRK | (Matasci et al., 2014) |
| [symbol] _ [symbol] _ [symbol] | [symbol] | EYRD | (Matasci et al., 2014) |
| [symbol] _ [symbol] _ [symbol] | [symbol] | XSSD | (Matasci et al., 2014) |
| [symbol] _ [symbol] _ [symbol] | [symbol] | WMLW | (Matasci et al., 2014) |
| [symbol] _ [symbol] _ [symbol] | [symbol] | ONLQ | (Matasci et al., 2014) |
| [symbol] _ [symbol] _ [symbol] | [symbol] | AAXJ | (Matasci et al., 2014) |
| [symbol] _ [symbol] _ [symbol] | [symbol] | CBJR | (Matasci et al., 2014) |
| [symbol] _ [symbol] _ [symbol] | [symbol] | WGET | (Matasci et al., 2014) |
| [symbol] _ [symbol] _ [symbol] | [symbol] | FVXD | (Matasci et al., 2014) |
| [symbol] _ [symbol] _ [symbol] | Genome | v1.1 | (Dohm et al., 2014) |
| [symbol] _ [symbol] _ [symbol] | [symbol] | CUTE | (Matasci et al., 2014) |
| [symbol] _ [symbol] _ [symbol] | SRA | SRX151423 | (Zhang et al., 2012) |
| [symbol] _ [symbol] _ [symbol] | [symbol] | SMMC | (Matasci et al., 2014) |
| [symbol] _ [symbol] _ [symbol] | Smith Lab | MJM1665 | (Brockington et al., 2015) |
| [symbol] _ [symbol] _ [symbol] | SRA | SRX302090 | (Fan et al., 2013) |
| [symbol] _ [symbol] _ [symbol] | [symbol] | CTYH | (Matasci et al., 2014) |
| [symbol] _ [symbol] _ [symbol] | [symbol] | CPKP | (Matasci et al., 2014) |
| [symbol] _ [symbol] _ [symbol] | [symbol] | JLOV | (Matasci et al., 2014) |
| [symbol] _ [symbol] _ [symbol] | Smith Lab | MJM1767 | (Brockington et al., 2015) |
| [symbol] _ [symbol] _ [symbol] | Genome | v1.0 | (Yagi et al, 2014) |
| [symbol] _ [symbol] _ [symbol] | Smith Lab | LCMsn | (Brockington et al., 2015) |
| [symbol] _ [symbol] _ [symbol] | [symbol] | RXEN | (Matasci et al., 2014) |
| [symbol] _ [symbol] _ [symbol] | [symbol] | SKNL | (Matasci et al., 2014) |
| [symbol] _ [symbol] _ [symbol] | [symbol] | OLES | (Matasci et al., 2014) |
| [symbol] _ [symbol] _ [symbol] | [symbol] | FZQN | (Matasci et al., 2014) |
| [symbol] _ [symbol] _ [symbol] SRA | SRA | SRX118777- | (Muyle et al., 2012) |
| | | SRX118782 | |
| [symbol] _ [symbol] _ [symbol] | SRA | SRX096120 | N/A¹ |
| [symbol] _ [symbol] _ [symbol] | [symbol] | TJES | (Matasci et al., 2014) |
| [symbol] _ [symbol] _ [symbol] | Smith Lab | MJM1652 | (Brockington et al., 2015) |
| [symbol] _ [symbol] _ [symbol] | SRA | SRX312294 | (Jensen et al., 2015) |
| [symbol] _ [symbol] _ [symbol] | [symbol] | WPYJ | (Matasci et al., 2014) |
| [symbol] _ [symbol] _ [symbol] | [symbol] | YNFJ | (Matasci et al., 2014) |
| [symbol] _ [symbol] _ [symbol] | [symbol] | RNBN | (Matasci et al., 2014) |
| [symbol] _ [symbol] _ [symbol] | [symbol] | SCAO | (Matasci et al., 2014) |
| [symbol] _ [symbol] _ [symbol] | [symbol] | NXTS | (Matasci et al., 2014) |
| [symbol] _ [symbol] _ [symbol] | [symbol] | WQUF | (Matasci et al., 2014) |
| [symbol] _ [symbol] _ [symbol] | Smith Lab | MJM1751 | (Brockington et al., 2015) |
| [symbol] _ [symbol] _ [symbol] | Smith Lab | MJM1741 | (Brockington et al., 2015) |
| [symbol] _ [symbol] _ [symbol] | Smith Lab | MJM1697 | (Brockington et al., 2015) |
| [symbol] _ [symbol] _ [symbol] | Smith Lab | SRX717838 | (Yang et al., 2015) |
| [symbol] _ [symbol] _ [symbol] | [symbol] | VJPU | (Matasci et al., 2014) |
| [symbol] _ [symbol] _ [symbol] | [symbol] | ZBTA | (Matasci et al., 2014) |
| [symbol] _ [symbol] _ [symbol] | [symbol] | JAFJ | (Matasci et al., 2014) |
| [symbol] _ [symbol] _ [symbol] | Smith Lab | SRX718572 | (Yang et al., 2015) |
| [symbol] _ [symbol] _ [symbol] | Smith Lab | MJM1714 | (Brockington et al., 2015) |
| [symbol] _ [symbol] _ [symbol] | Smith Lab | SRX718384 | (Yang et al., 2015) |
| [symbol] _ [symbol] _ [symbol] | [symbol] | JGAB | (Matasci et al., 2014) |
| [symbol] _ [symbol] _ [symbol] | Smith Lab | MJM1771 | (Brockington et al., 2013) |
| [symbol] _ [symbol] _ [symbol] | Smith Lab | SRX718389 | (Yang et al., 2015) |
| [symbol] _ [symbol] _ [symbol] | Smith Lab | SFB29 | (Brockington et al., 2015) |
| [symbol] _ [symbol] _ [symbol] | [symbol] | RUUB | (Matasci et al., 2014) |
| [symbol] _ [symbol] _ [symbol] | Smith Lab | MJM1649 | (Brockington et al., 2015) |
| [symbol] _ [symbol] _ [symbol] | [symbol] | SFKQ | (Matasci et al., 2014) |
| [symbol] _ [symbol] _ [symbol] | [symbol] | AZBL | (Matasci et al., 2014) |
| [symbol] _ [symbol] _ [symbol] | [symbol] | BKQU | (Matasci et al., 2014) |
| [symbol] _ [symbol] _ [symbol] | [symbol] | MRKX | (Matasci et al., 2014) |
| [symbol] _ [symbol] _ [symbol] | Smith Lab | SFB31 | (Brockington et al., 2015) |
| [symbol] _ [symbol] _ [symbol] | Smith Lab | SRX718277 | (Yang et al., 2015) |
| [symbol] _ [symbol] _ [symbol] | Smith Lab | SRX718486 | (Yang et al., 2015) |
| [symbol] _ [symbol] _ [symbol] | [symbol] | WOBD | (Matasci et al., 2014) |
| [symbol] _ [symbol] _ [symbol] | Smith Lab | MJM1811 | (Brockington et al., 2015) |
| [symbol] _ [symbol] _ [symbol] | SRA | SRX112838 | N/A¹ |
| [symbol] _ [symbol] _ [symbol] | [symbol] | FYSJ | (Matasci et al., 2014) |
| [symbol] _ [symbol] _ [symbol] | SRA | SRX079484 | (Hao et al., 2012) |
| [symbol] _ [symbol] _ [symbol] | SRA | SRX621187 | N/A¹ |
| [symbol] _ [symbol] _ [symbol] | SRA | SRX286365 | N/A¹ |
| [symbol] _ [symbol] _ [symbol] | SRA | ERX190940 | N/A¹ |
| [symbol] _ [symbol] _ [symbol] | SRA | ERX190941, | N/A¹ |
| | | ERX190942 | |
| [symbol] _ [symbol] _ [symbol] | [symbol] | LDEL | (Matasci et al., 2014) |
| [symbol] _ [symbol] _ [symbol] | [symbol] | LLQV | (Matasci et al., 2014) |
| [symbol] _ [symbol] _ [symbol] | [symbol] | CPLT | (Matasci et al., 2014) |
| [symbol] _ [symbol] _ [symbol] | [symbol] | UQCB | (Matasci et al., 2014) |
| [symbol] _ [symbol] _ [symbol] | [symbol] | EZGR | (Matasci et al., 2014) |
| [symbol] _ [symbol] _ [symbol] | [symbol] | IWIS | (Matasci et al., 2014) |
| [symbol] _ [symbol] _ [symbol] | [symbol] | GCYL | (Matasci et al., 2014) |
| [symbol] _ [symbol] _ [symbol] | [symbol] | GIWN | (Matasci et al., 2014) |
| [symbol] _ [symbol] _ [symbol] | [symbol] | CVDF | (Matasci et al., 2014) |
| [symbol] _ [symbol] _sp | [symbol] | LKKX | (Matasci et al., 2014) |
| [symbol] _ [symbol] _ [symbol] | SRA | SRX099851, | N/A¹ |
| | | SRX105466 | |
| [symbol] _ [symbol] _ [symbol] | SRA | All [symbol] runs in | (Wang et al., 2014) |
| | | PRJNA170420 | |

Outgroups

| | | | |
|---|---|---|---|
| [symbol] _ [symbol] | Genome | Accessed | (Goodstein et al., 2012) |
| | | May 28, 2014 | |
| [symbol] _ [symbol] | Genome | Accessed | (Goodstein et al., 2012) |
| | | Apr. 21, 2015 | |
| [symbol] _ [symbol] | Genome | Accessed | (Goodstein et al., 2012) |
| | | May 28, 2014 | |
| [symbol] _ [symbol] | Genome | Accessed | (Goodstein et al., 2012) |
| | | Apr. 21, 2015 | |

¹N/A, not available. [symbol]
indicates data missing or illegible when filed

### Subcellular Localization of GFP-Fused ADH Enzymes

The subcellular localization experiments of GFP-fused ADH enzymes were conducted as we described previously (Schenck et al., 2015).

### Accession Numbers

The Genbank accession numbers for the sequences mentioned in this article are: BvADHβ W357B red beet variety (KY207366), BvADHβ Boltardy red beet variety (MF346292), BvADHβ BigBuck sugar beet variety (KY207367), BvADHβ Touch Stone yellow beet variety (KY207368), BvADHβ Blankoma white beet variety (KY207369), BvADHβ Sea beet PI562585 variety (KY207370), BvADHα Big Buck sugar beet variety (KY207371), BvADHα W357B red beet variety (KY207372), BvADHα Boltardy red beet variety (MF346291), BvADHα Blankoma white beet variety (KY207373), BvADHα Touch Stone yellow beet variety (KY207374), BvADHα Sea beet PI562585 variety (KY207375), SoADHβ (KY207376), SoADHα (KY207378), NaADHβ (KY207377), MjADHα (KU881770), RhADHα (KY207379), PoADHα (KY207380), SmADHa (KY274179), PpADHα (KY274180), and HlADHα (KY274181).

### Results

B. vulgaris has Two ADH Enzymes.

To first investigate how B. vulgaris synthesizes Tyr, protein crude extracts of red beet leaf and root/stem tissues were analyzed for ADH and PDH activity, the production of Tyr or HPP from arogenate or prephenate, respectively. Tyr was produced from arogenate in the red beet extracts of both leaves and roots/stems (FIG. 3A) similar to soybean leaf extract, which was previously shown to have both ADH and PDH activity (Schenck et al., 2015). On the other hand, unlike the soybean leaf extract, HPP production was not detected in the leaf and root/stem extracts of red beet (FIG. 3B). These results showed that red beet has ADH but not PDH activity.

To identify the gene(s) responsible for the ADH activity in B. vulgaris, previously reported plant and microbial ADH and PDH genes (Bonvin et al., 2006; Hudson et al., 1984; Legrand et al., 2006; Rippert & Matringe, 2002a,b; Schenck et al., 2015, FIG. 2B) were used to BLAST against the genome of sugar beet, another cultivar of B. vulgaris (Dohm et al., 2014) (assembly v.1.2 molgen.mpg.de). Two B. vulgaris sequences homologous to these ADH and PDH genes were found on chromosome 8 of the B. vulgaris genome 25.3 kbp apart (FIG. 2A). They were more similar to plant ADHs and PDHs (59 to 61% similarity at amino acid levels) than bacterial ones (24 to 40% similarity, FIG. 2B). Within plants, the two ADH candidate genes from B. vulgaris both belong to the canonical ADH clade containing Arabidopsis ADHs (Rippert & Matringe, 2002a,b), rather than the non-canonical clade containing legume PDHs (Schenck et al., 2015; 2017), and appear to be derived from a recent duplication within the order Caryophyllales.

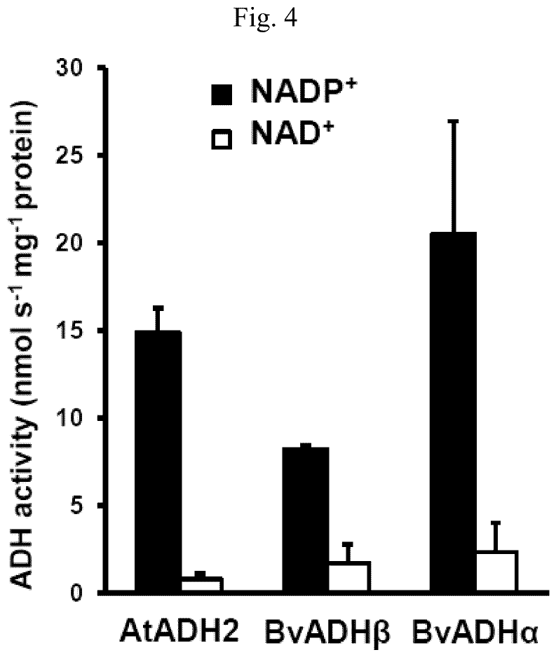

For biochemical characterization, these two putative BvADHs were expressed in E. coli as recombinant enzymes, which were further purified using affinity chromatography and subjected to ADH and PDH assays. Both of the beet recombinant enzymes showed ADH activity (i.e. the production of Tyr from arogenate, FIG. 1B) and strongly preferred NADP⁺ over NAD⁺ (FIG. 4) similar to other plant ADH enzymes and activities (Gaines et al., 1986; Rippert & Matringe, 2002a,b). On the other hand, neither of the beet enzymes exhibited detectable PDH activity (FIG. 3C), which is consistent with the lack of PDH activity in beet tissues (FIG. 3B) and also confirmed the absence of E. coli PDH contamination (Hudson et al., 1984). Therefore, these two genes were designated as B. vulgaris arogenate dehydrogenases (BvADHα and BvADHβ).

Both BvADHs are Plastid Localized but Only BvADHα Expression is Correlated with Betalain Pathway Genes.

Most plant enzymes involved in the aromatic amino acid pathways are localized within the plastids (Dal Cin et al., 2011; Maeda & Dudareva, 2012; Rippert et al., 2009), and both BvADH proteins also have a predicted N-terminal plastid transit peptide (FIGS. 5A-5D). To experimentally determine the subcellular localization of BvADHs, a green fluorescent protein (GFP) was fused to the C-terminal of BvADHs, expressed in Arabidopsis protoplasts, and analyzed for their localization using confocal microscopy. The fluorescence signal of GFP fused with BvADHα or BvADHβ overlapped with chlorophyll autofluorescence, which was different from the free GFP control and similar to GFP fused with plastidic Arabidopsis ADH (Rippert et al., 2009) (AtADH2, FIG. 1C). These results suggest that both BvADHs are targeted to the plastids and that Tyr is mainly produced by the plastidic arogenate pathway in B. vulgaris.

To examine expression patterns of BvADHs, especially in comparison to the betalain pathway genes, expression levels of BvADHα and BvADHβ were analyzed and compared with those of DODAα, CYP76AD1α, and BvMYB1 in cotyledon and hypocotyl tissues of sugar and red beets (FIG. 1D). Consistent with previous studies (Hatlestad et al., 2012; 2015), DODAα and CYP76AD1α, as well as BvMYB1 transcription factor, were much more highly expressed in red than sugar beet. Interestingly, BvADHα expression showed similar trends and was significantly higher in red than sugar beet in both cotyledon and hypocotyl tissues. On the other hand, BvADHβ expression levels were very similar between genotypes in both tissue types (FIG. 1D). These results showed that expression of BvADHα, but not BvADHβ, is correlated with those of betalain pathway genes in B. vulgaris.

### BvADHα but not BvADHβ Exhibits Relaxed Sensitivity to Tyr

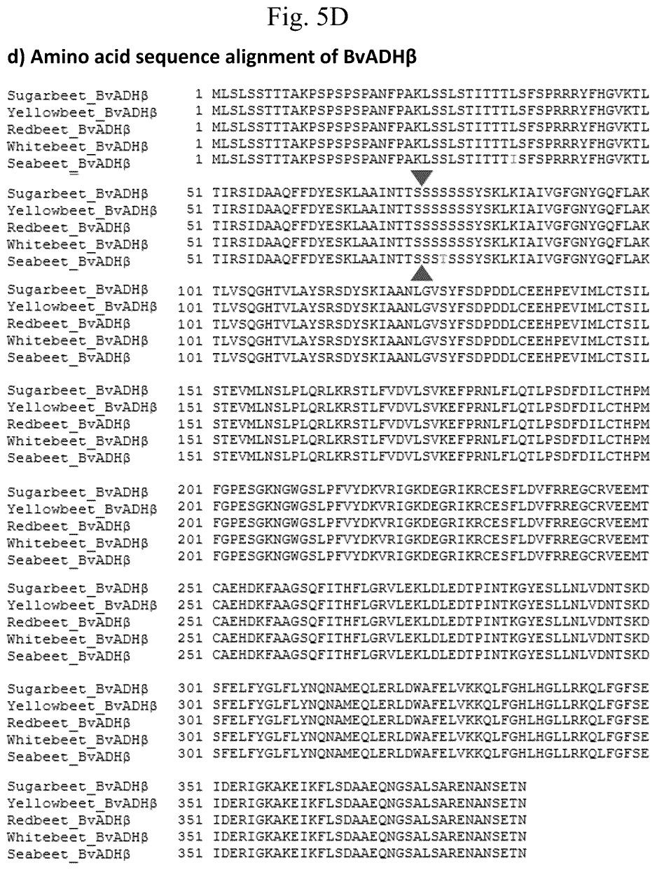

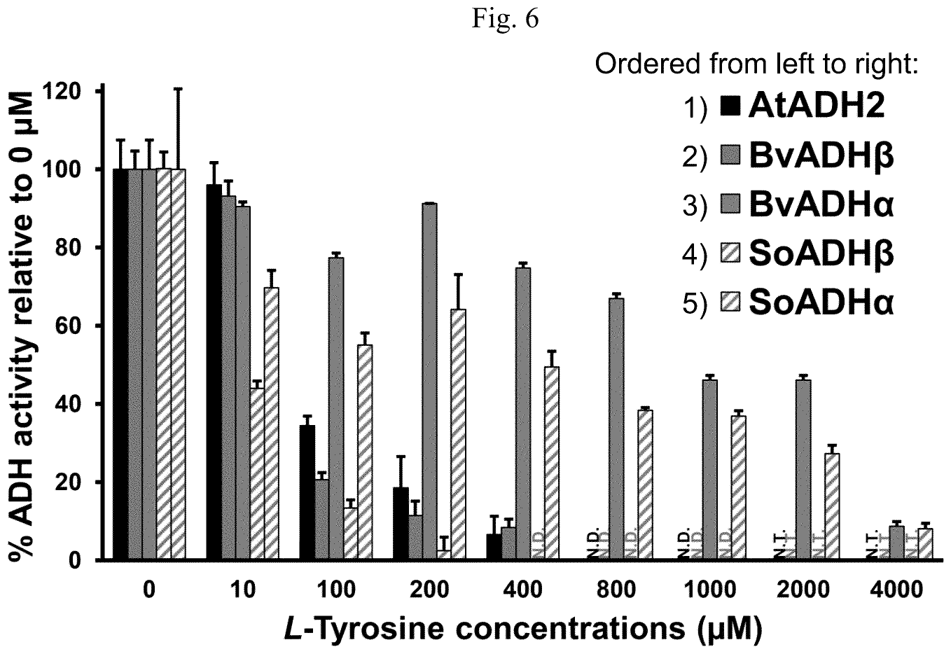

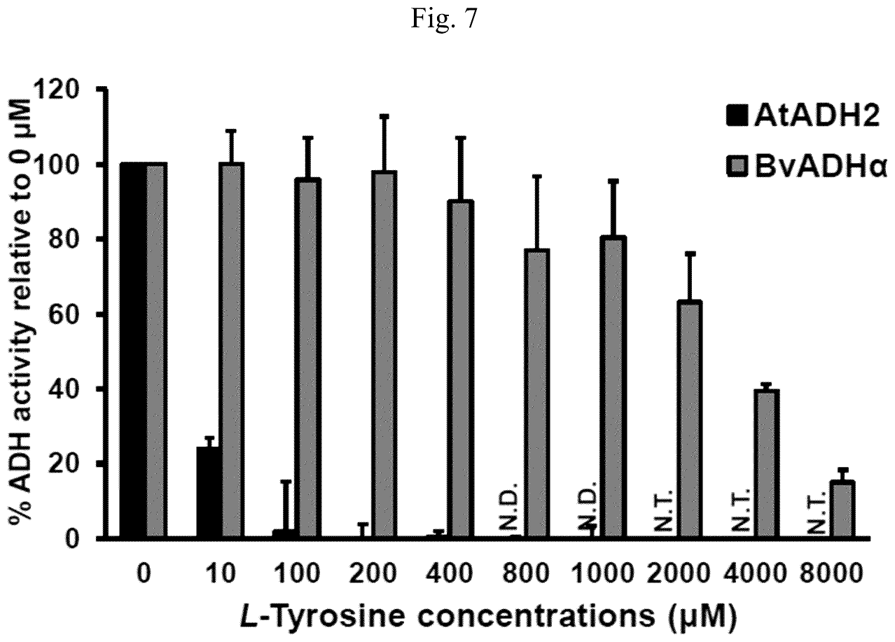

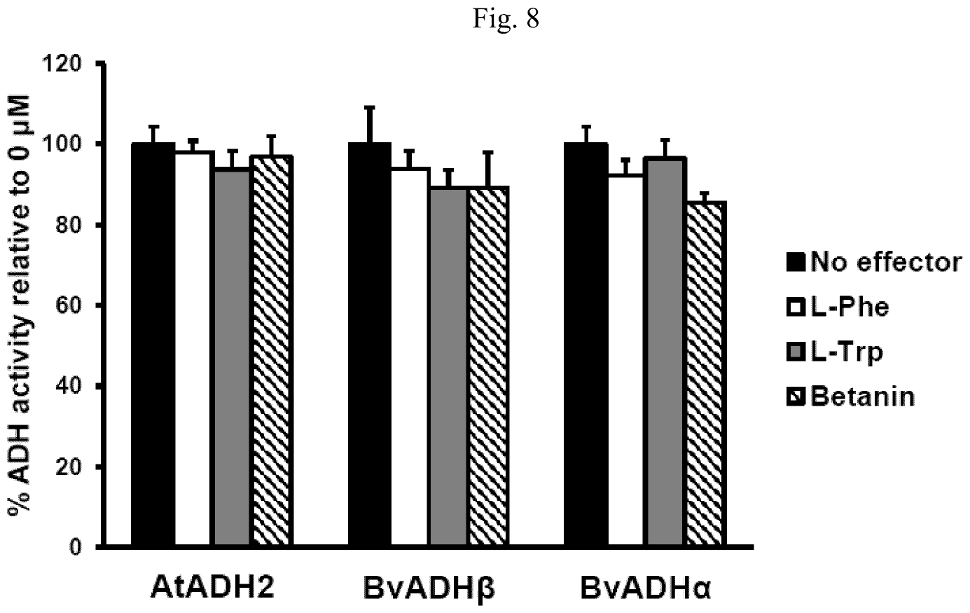

Both ADH and PDH enzymes are usually inhibited by Tyr in most organisms (Bentley, 1990; Connelly & Conn, 1986; Gaines et al., 1982; Rippert & Matringe, 2002a,b; Sun, 2009). To determine if the BvADHs are also feedback regulated by Tyr, ADH activity of the recombinant BvADH enzymes were analyzed in the presence and absence of Tyr as an effector molecule. The ADH activity of glutathione S-transferase (GST)-tagged BvADHβ was inhibited by 80% and 100% in the presence of 100 μM and 1 mM Tyr, respectively (FIG. 6), similar to the Tyr-sensitive Arabidopsis AtADH2 (Rippert & Matringe, 2002a,b). In contrast, ADH activity of BvADHα was reduced only by half at 1 mM Tyr (FIG. 6). Similar results were obtained for histidine (His)-tagged ADH enzymes, where BvADHα showed much less sensitivity to Tyr than AtADH2 (FIG. 7), though the expression of His-tagged BvADHβ was not successful. Other aromatic amino acids (Phe and tryptophan) as well as betanin, the major betacyanin accumulated in red beet, did not significantly reduce the ADH activity of BvADHα, BvADHβ, or AtADH2 at 1 mM (FIG. 8). These results revealed that BvADHα, but not BvADHβ, has relaxed sensitivity to Tyr inhibition.

### Heterologous Expression of BvADHα but not BvADHβ Increase Tyr Accumulation in Plants.

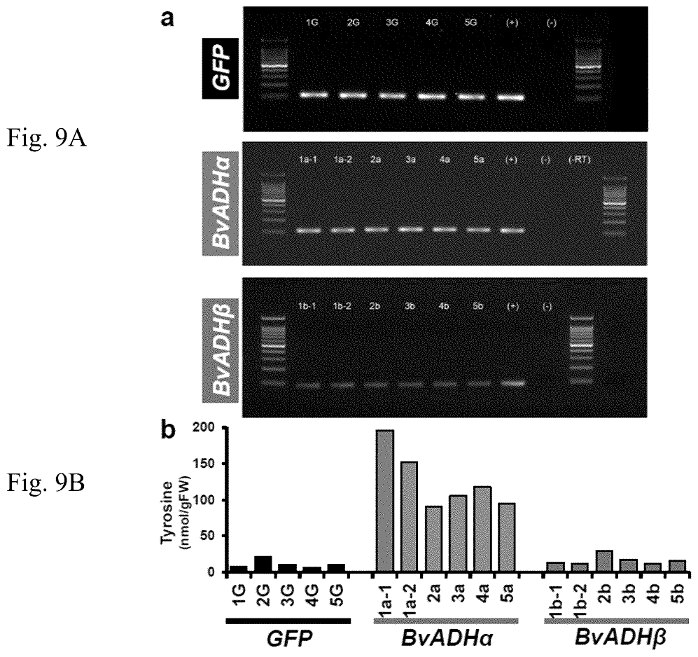

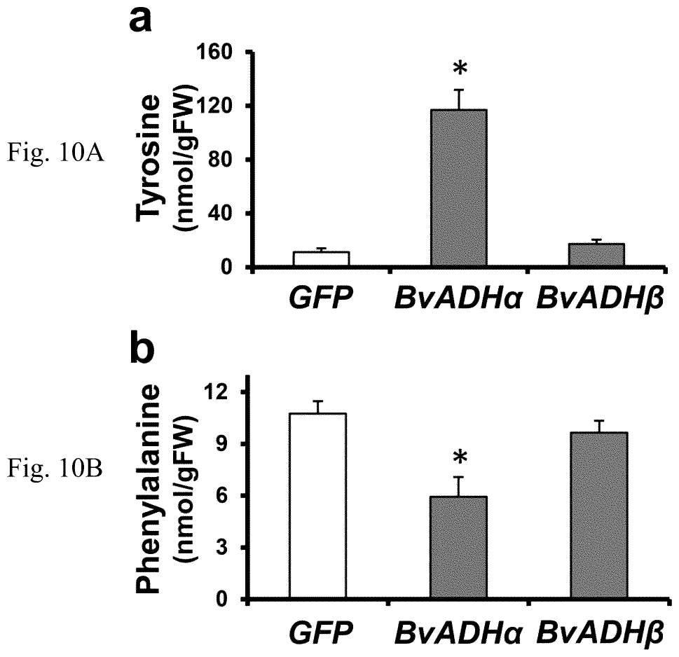

To test if BvADHα having relaxed sensitivity to Tyr can enhance the production of Tyr in planta, BvADHα and BvADHβ were transiently expressed in N. benthamiana through Agrobacteria infiltration (FIG. 9A, Sparkes et al., 2006) and their impacts on Tyr production were analyzed. A control vector expressing GFP was also infiltrated as a negative control (FIG. 9A). BvADHα expression resulted in >10-fold increase in Tyr levels relative to the GFP control, while the increase of Tyr due to BvADHβ expression was not significantly different (FIGS. 10A & 9B, Table 3). Interestingly, phenylalanine (Phe) levels were decreased significantly under BvADHα, but not BvADHβ expression (FIG. 10B). Other amino acid levels were largely unaffected by BvADHα or BvADHβ expression (Table 3). These results demonstrate that BvADHα expression leads to elevated accumulation of Tyr in planta.

TABLE 3
Amino Acid levels of Nicotiana benthamiana leaves expressing
GFP, BvADHα, BvADHβ. Agrobacteria carrying the 35S::GFP,
35S::BvADHα, or 35S::BvADHβ construct were infiltrated
to Nicotiana benthamiana leaves and the levels of amino acids were
analyzed after three days post-infiltration. Data are mean ± s.e.m.
(nmol/gFW, n = 5 biological replications). Asterisks denote values
significantly different from the control 35S::GFP sample (Student t-test,
p < 0.01). Tryptophan, lysine, cysteine, and histidine levels were
below quantification threshold.

| Amino Acids | 35S::GFP | 35S::BvADHα | 35S::BvADHβ |
|---|---|---|---|
| alanine | 99.8 ± 15.5 | 93.0 ± 14.8 | 88.1 ± 20.0 |
| glycine | 15.5 ± 1 | 17.5 ± 2.1 | 13.6 ± 0.2 |
| valine | 23.9 ± 9.7 | 23.8 ± 8.3 | 22.1 ± 8.4 |
| leucine | 21.3 ± 10.4 | 21.8 ± 9.2 | 18.8 ± 8.3 |
| isoleucine | 13.8 ± 7 | 13.3 ± 5.7 | 13.3 ± 6.7 |
| proline | 154.8 ± 67.4 | 126.7 ± 56.3 | 137.3 ± 75.4 |
| methionine | 2.8 ± 0.4 | 3.1 ± 0.4 | 2.6 ± 0.2 |
| serine | 57.4 ± 8 | 58.6 ± 11.7 | 43.9 ± 3.9 |
| threonine | 69.4 ± 7.5 | 67.8 ± 8.6 | 58.1 ± 6.5 |
| phenylalanine | 10.8 ± 0.7 | 5.9 ± 1.2* | 9.7 ± 0.7 |
| aspartic acid | 173.5 ± 45.5 | 176.8 ± 40.6 | 132.7 ± 41.5 |
| glutamic acid | 941.6 ± 45.8 | 968.1 ± 91.6 | 746.4 ± 111.4 |
| omithine^{a} | 54.9 ± 1.6 | 56.2 ± 2.4 | 48.4 ± 2.9 |
| asparagine | 6.8 ± 1.2 | 6.9 ± 1.5 | 4.9 ± 1.0 |
| glutamine | 345.2 ± 116.1 | 348.7 ± 138.4 | 291.3 ± 107.7 |
| tyrosine | 11.2 ± 2.8 | 116.8 ± 15.1* | 17.2 ± 3.2 |

^{a}Arginine was quantified as its non-enzymatic degradation product omithine.

### BvADHα Orthologs Emerged During the Evolution of Betalain Pigmentation in Caryophyllales.

Domestication has modified metabolic traits in various crops (Hanson et al., 1996; Rapp et al., 2010; Rong et al., 2014). Thus, we hypothesized that the BvADHα enzyme with relaxed Tyr regulation was selected during domestication and intensification of color in table beets, that have been used at least since the Roman times (Biancardi et al., 2012; Dohm et al., 2014). To test this hypothesis, the nucleotide and protein sequences of BvADHα (and BvADHβ) were compared among different domesticated beets, red beet (W357B), sugar beet (Big Buck), yellow beet (Touch Stone), and white beet (Blankoma), as well as their wild relative, sea beet (Biancardi et al., 2012) (Beta vulgaris subsp. maritima). Several single nucleotide polymorphisms (SNPs) were detected among different lines in both BvADHα and BvADHβ (FIGS. 5A, 5B). However, only a few of them affected the amino acid sequences and were within and near the N-terminal signal peptide of BvADHα and BvADHβ, respectively (FIGS. 5C, 5D). Thus, the mature enzyme regions of BvADHα were unaltered during domestication.

To further test if the ADHα enzymes with reduced Tyr sensitivity are restricted to the species B. vulgaris, the corresponding genes for BvADHα and BvADHβ were cloned from a closely related species within the same Amaranthaceae family, spinach (Spinacia oleracea), whose draft genome is available (molgen.mpg.de). Spinach ADHα and ADHO orthologs (SoADHα and SoADHβ) had 77 and 83% identity at amino acid levels to the corresponding BvADHs in the mature enzymatic regions. The recombinant enzymes of spinach ADHs showed similar Tyr sensitivity to beet ADHs: SoADHα, but not SoADHβ, exhibited reduced Tyr sensitivity (FIG. 6). These results suggest that the reduced Tyr sensitivity of BvADHα at least at the enzyme level was not the result of selection during domestication of beet cultivars, but was already present in the common ancestor of the beet and spinach ADHα enzymes.

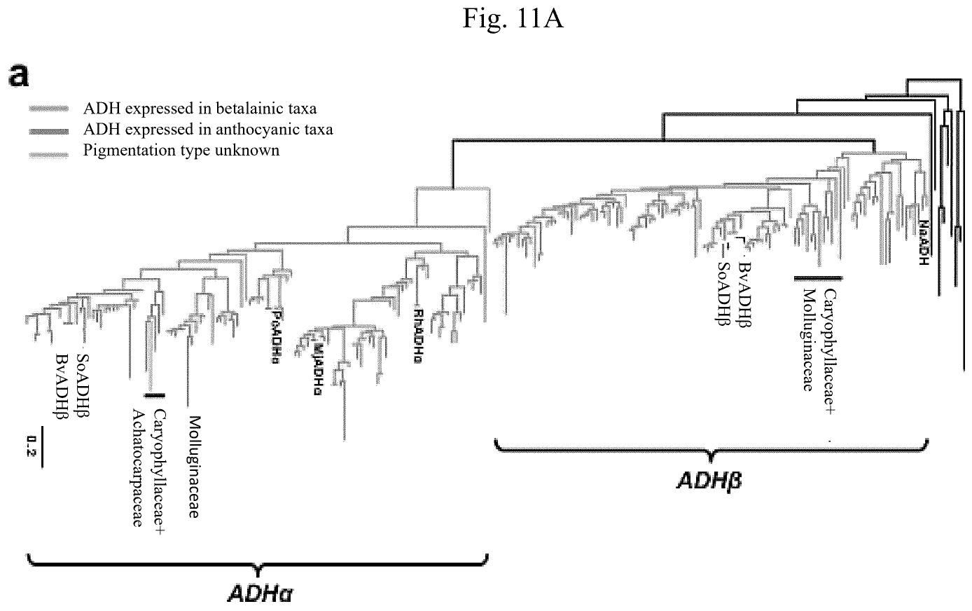

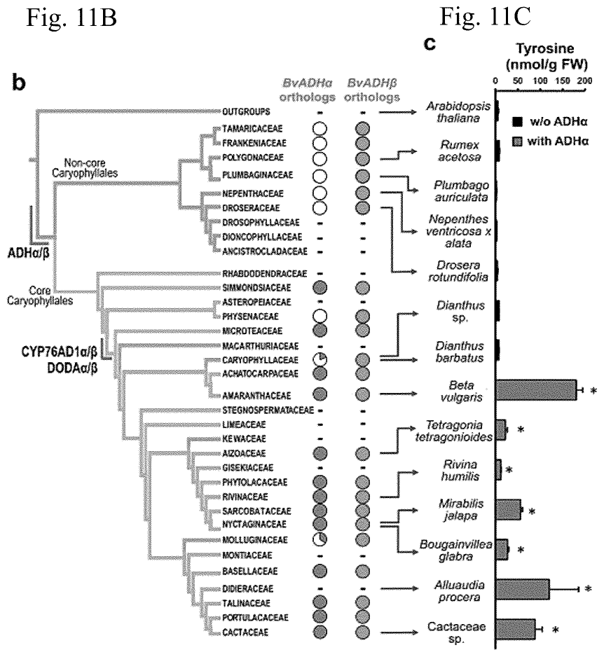

To determine the origin and molecular evolution of BvADHα, we mined genome and transcriptomic data across the Caryophyllales for ADH orthologs and performed a phylogenetic analysis (FIG. 11A). The results indicate that a gene duplication event on the branch leading to stem Caryophyllales produced ADHα and ADHβ lineages. While ADHβ orthologs were expressed across the entire Caryophyllales, expression of ADHα closely parallels betalain production in Caryophyllales. ADHα expression is undetectable from the anthocyanic clade that diverged prior to the earliest inferred origin of betalain synthesis (hereafter referred to as non-core Caryophyllales; Brockington et al., 2009). Two families in the Caryophyllales, Molluginaceae and Caryophyllaceae have reverted from betalain to anthocyanin pigmentation (Brockington et al., 2011, 2015). Presence of the ADHα orthologs in the transcriptomes of Molluginaceae and Caryophyllaceae was much less common than the presence of BvADHβ (FIGS. 11A, 11B). Thus the presence of ADHα, but not ADHβ, closely mirrors the distribution of betalain pigmentation across Caryophyllales, similar to the pattern in two other genes of the betalain pathway, CYP76AD1α and DODAα (Brockington et al., 2015).

Betalain-Producing Species have Deregulated BvADHα Enzyme and Elevated Tyr Levels.

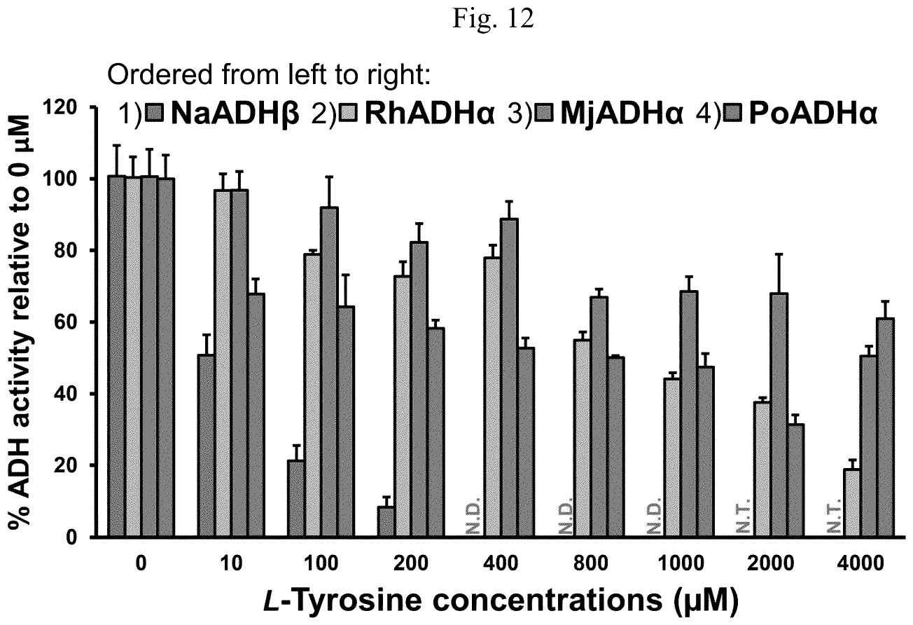

To further test experimentally if ADHα orthologs across Caryophyllales share the unique property of reduced Tyr inhibition, ADH genes from representative members of Caryophyllales (Brockington et al., 2011) were cloned and the Tyr sensitivity of encoded enzymes was evaluated. An ADHβ enzyme from the anthocyanin-producing non-core Caryophyllales, Nepenthes ventricosa x alata (NaADHβ, Nepenthaceae, FIG. 11B), was strongly inhibited by Tyr (FIG. 12) similar to beet and spinach ADHO (FIG. 6). On the other hand, ADHα orthologs from betalain-producing families, Rivina humilis (RhADHa, Rivinaceae), Mirabilis jalapa (MjADHa, Nyctaginaceae), and Portulaca oleracea (PoADHa, Portulacaceae), all shared relaxed Tyr inhibition and retained 42% to 68% of ADH activity even at 1 mM Tyr (FIG. 12).

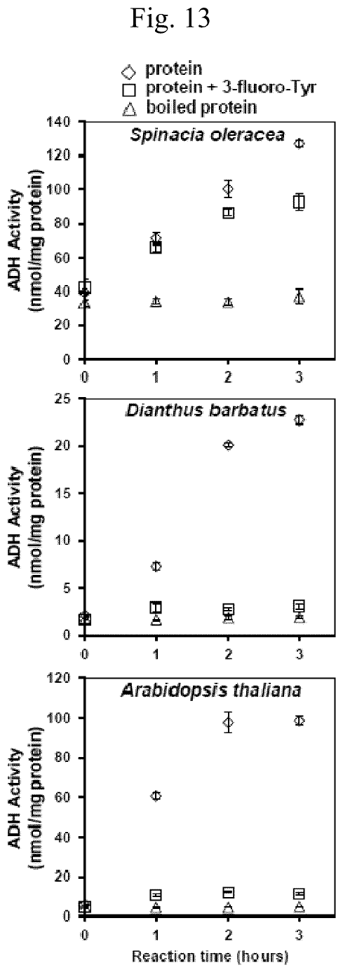

To test if Tyr-insensitivity of the recombinant ADHα enzyme is also detectable in vivo, Tyr sensitivity of leaf ADH activity was analyzed from species containing ADHα (i.e. spinach) and ones lacking ADHα [i.e. Arabidopsis thaliana; Dianthus barbatus, Caryophyllaceae]. Spinach rather than beet was used due to its cleaner background during HPLC-based enzyme assay. As shown in Table 4 and FIG. 13, ADH activity of Arabidopsis and Dianthus barbatus tissues was strongly inhibited (92-95%) by 0.5 mM of Tyr effector, whereas that of spinach was much more resistant to Tyr inhibition (only ˜21% inhibited), consistent with the presence of SoADHα with relaxed sensitivity to Tyr (FIG. 6).

TABLE 4
Tyr sensitivity of ADH activity from plant tissue extracts. Total
protein extracts of spinach, Dianthus barbatus, and Arabidopsis
leaf tissues were used to analyze ADH activity in the
presence and absence of 0.5 mM Tyr analog (3-fluoro-Tyr),
which were used to calculate percent inhibition. ADH activity
was measured with 1 mM arogenate substrate
and 1 mM NADP⁺ cofactor during
2 hr incubation (see FIG. 13). Data are means ± s.e.m. (n = 4).

| | ADH activity (nmol/mg protein) | | |
|---|---|---|---|
| | 0 mM | 0.5 mM | inhibition |
| species | 3-fluoro-Tyr | 3-fluoro-Tyr | (%) |
| Spinach oleracea | 66.4 ± 5.0 | 52.7 ± 1.9 | 20.7% |
| Dianthus barbatus | 18.1 ± 0.3 | 0.9 ± 0.2 | 95.0% |
| Arabidopsis thaliana | 93.5 ± 5.2 | 7.8 ± 0.5 | 91.6% |

To further test if the presence of deregulated ADHα leads to increased Tyr accumulation in betalain-producing species, Tyr levels were quantified in young leaves of a variety of Caryophyllales species with or without ADHα and also in Arabidopsis thaliana as a comparison. Anthocyanin-producing species from non-core Caryophyllales (e.g. Nepenthes ventricosa x alata) and Caryophyllaceae (e.g. Dianthus barbatus) had Tyr levels (2.1 to 8.8 nmol/gFW) comparable to that of Arabidopsis (5.3 nmol/gFW). On the other hand, while large variations were observed, betalain-producing ADHα-containing species all had significantly higher Tyr levels (from 12 to 180 nmol/gFW) than Arabidopsis (FIG. 11C). These results demonstrate that betalain-producing species have ADHα with relaxed sensitivity to Tyr inhibition and accumulate elevated levels of Tyr.

ADHα Orthologs Underwent Relaxed Selection and Gene Loss in Lineages that have Reverted from Betalain to Anthocyanin Pigmentation

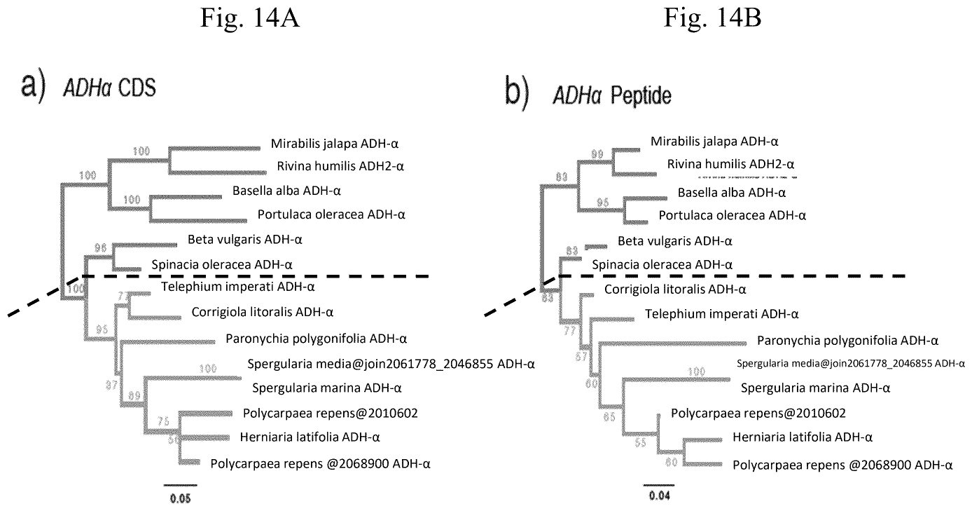

Interestingly, when ADHα orthologs were recovered from Caryophyllaceae or Molluginaceae transcriptomic data, they were often recovered in partial sequences, indicating general low abundance. Within the Caryophyllaceae, ADHα orthologs was only detected in the subfamily Paronychioideae (Greenberg & Donoghue, 2011), which forms a grade paraphyletic to the rest of the family. To test for relaxed selection in anthocyanic lineages we further examined a subset of ADHα orthologs with sequences either verified by Sanger sequencing or by transcriptome read mapping and manual inspection of read coverage. Although no obvious acceleration of substitution was observed in Caryophyllaceae from nucleotide coding sequences (CDS, FIG. 14A), there was apparent acceleration in their amino acid sequences (FIG. 14B). Furthermore, the dN/dS ratio in Caryophyllaceae ADHα (0.166) was elevated compared to the rate among betalain-producing ADHα (0.0743) under the Partitioned MG94xREV Model, assuming homogenous synonymous and nonsynonymous rates across sites. In addition, we found evidence of relaxed selection (as opposed to intensification of positive selection) that contributes to the increase in nonsynonymous rate in Caryophyllaceae under the RELAX framework (p=5.6E-8, Table 5) (Wertheim et al., 2014). Moreover, the genome assembly of the anthocyanic carnation (Dianthus caryophyllus, Caryophyllaceae subfamily Caryophylloideae that nested within subfaily Paronychioideae, Greenberg & Donoghue, 2011; Yagi et al., 2014) lacked ADHα ortholog and only contained ADHβ ortholog, suggesting complete gene loss of ADHα in the subfamily Caryophylloideae (Greenberg & Donoghue, 2011). Species within the anthocyanic Caryophyllaceae, therefore, exhibit the transition from relaxed selection to gene loss of ADHα orthologs, which associates with the loss of betalain pigmentation in Caryophyllaceae.

TABLE 5
RELAX analysis support the acceleration in amino acid substitution in Caryophyllales
is due to relaxed purifying selection, instead of intensified positive selection

| | | | | | | ω1 (purifying | | ω2 (nearly | | ω3 (positive | |
|---|---|---|---|---|---|---|---|---|---|---|---|
| Model | log L | # par. | AlCc | Ltree | Branch set | selection) | | neutral) | | selection) | |
| Partitioned | −5484.8 | 38 | 11046.5 | 2.23 | Reference | 0.0743 (100%) | | | | | |
| MG94xREV | | | | | Test | 0.186 (100%) | | | | | |
| Null | −5374.3 | 41 | 10831.7 | 11.9 | Reference | 0.00 | (83%) | 0.550 | (15%) | 30.9 | (1.4%) |
| | | | | | Test | 0.00 | (83%) | 0.550 | (15%) | 30.9 | (1.4%) |
| Alternative | −5359.6 | 42 | 10804.2 | 84.5 | Reference | 0.00598 | (91%) | 0.650 | (7.9%) | 540 | (1.5%) |
| | | | | | Test | 0.0646 | (91%) | 0.794 | (7.9%) | 29.0 | (1.5%) |

K = 0.54. Test for selection relaxation (K < 1) was significant (p = 5.6e−8, LR = 29.48)

### Discussion

This study found that B. vulgaris has ADH but no PDH enzymes or activity (FIG. 1B, FIGS. 3, 4). This is similar to most plants (Connelly & Conn, 1986; Gaines et al., 1982; Rippert & Matringe, 2002a,b) but different from legumes that have both ADH and PDH (Rubin & Jensen, 1979; Schenck et al., 2015; 2017; Siehl, 1999). Thus, B. vulgaris synthesizes Tyr via the ADH pathway that occurs within the plastids (Rippert et al., 2009) (FIG. 1C). We also found that B. vulgaris possesses two paralogous genes encoding the ADH enzymes, namely ADHα and ADHβ. Interestingly, ADHα but not ADHβ exhibited relaxed sensitivity to Tyr inhibition (FIG. 6). Although recent studies reported that the legume PDH enzymes are also Tyr insensitive (Schenck et al., 2015; 2017), BvADHα and legume PDHs have two major differences. First, legume PDHs are localized in the cytosol (Schenck et al., 2015), whereas BvADHα (and BvADHβ) was targeted to the plastids (FIG. 1C). Second, legume PDHs completely lost Tyr sensitivity (Schenck et al., 2015) but BvADHα was still inhibited by Tyr at higher concentrations (FIG. 6, FIG. 7). The maintenance of inhibition at higher concentration is likely necessary because Phe biosynthesis is also localized within the plastids, and thus BvADHα is directly competing for the arogenate substrate with Phe biosynthesis (FIG. 1A). Complete loss of ADH regulation by Tyr would, therefore, deplete Phe and essential Phe-derived compounds (e.g., proteins, lignin).

Other insensitive ADH/PDH enzymes have been previously found in microorganisms (Legrand et al., 2006) and the structural analyses of Tyr sensitive and insensitive enzymes identified histidine 217 as a possible residue responsible for its Tyr sensitivity (Legrand et al., 2006; Sun et al., 2009). Also, phylogeny-guided structure-function analysis revealed that converting a single active site aspartate 222 residue into a non-acidic residue played a key role in the evolution of the legume PDH enzymes and simultaneously introduced prephenate substrate specificity and Tyr insensitivity (Schenck et al., 2017). However, the corresponding histidine and aspartate residues are still present in BvADHα (FIG. 15), suggesting that different mechanisms, and as yet unidentified residues are involved in the relaxed Tyr sensitivity of BvADHα.

Previous analyses of molecular evolution of DODAα and CYP76AD1α, two enzymes which convert Tyr into betalains (Christinet et al., 2004; Gandia-Herrero & Garcia-Carmona, 2012; Hatlestad et al., 2012), revealed that both of these genes arose through gene duplication, just prior to the origin of betalain pigmentation in Caryophyllales (Brockington et al., 2015). Similarly, this study found that ADHα orthologs arose by gene duplication, prior to the emergence of DODAα and CYP76AD1α (FIGS. 11A and 11B), intimately associated with the origin of betalain pigmentation. One of the duplicated copies, ADHa, underwent neofunctionalization and became much less sensitive to Tyr inhibition, which is the key regulatory mechanism of Tyr biosynthesis (Maeda & Dudareva, 2012; Rippert & Matringe, 2002a,b). ADHα enzymes with relaxed Tyr sensitivity are maintained in all betalain-producing species of Caryophyllales, at least the ones that we analyzed (FIGS. 6 and 12). Furthermore, the expression pattern of BvADHα is distinct from that of BvADHβ and similar to those of the betalain biosynthetic genes (DODAα and CYP76AD1α) and MYB1 transcription factor (FIG. 1D), suggesting that the alteration of ADHα enzyme property was accompanied by changes in its expression profile. Although similar examples of biochemical and transcriptional changes during the evolution of plant specialized metabolic enzymes/genes have been reported (Kajikawa et al., 2017; Moghe & Last, 2015; Panchy et al., 2016; Weng et al., 2012; Xu et al., 2017), here we revealed a unique example of coordinated evolution of primary amino acid pathway (i.e. Tyr biosynthesis) and its downstream specialized metabolism (i.e. betalain biosynthesis).

In the anthocyanic Caryophyllaceae, the transition of betalain pigmentation to anthocyanin pigmentation was associated with down-regulation, relaxed natural selection, and deletion of ADHα (FIGS. 11, and 14, Table 5). Similar down-regulation and deletion of genes were also observed during the loss of flower petals (Zhang et al., 2013) and arbuscular mycorrhizal symbiosis (Delaux et al., 2014) in various plant lineages. Together these lines of evidence suggest that maintenance of the ADHα is superfluous, following loss of betalain pigmentation. The ultimate cause of reversion of betalain to anthocyanin pigmentation in multiple lineages within the core Caryophyllales is currently unknown. It may be due to a number of factors, including: i) metabolic cost of nitrogen-containing alkaloid betalain pigments, ii) shift in pollinator populations that are attracted by unique spectra (e.g. blue) of some anthocyanins, iii) increased demand for other Phe-derived compounds (e.g. tannins, flavonoids), or iv) simple genetic drift enabled by the presence of still intact Phe, phenylpropanoid, core flavonoid pathways in betalain-producing plants (Brockington et al, 2011;, Shimada et al., 2005; Xu et al., 2016).

A mechanism underlying the mutually exclusive distribution of betalain and anthocyanin pigments has long fascinated evolutionary biologists (Brockington et al, 2011; Des Marais, 2015). Our analyses now provide one possible explanation. The relaxation of the Tyr-mediated feedback inhibition may direct more carbon flow towards Tyr, and away from Phe biosynthesis (FIG. 1A), as demonstrated by increased Tyr and decreased Phe levels upon transient expression of ADHα (FIG. 10). This may create a surplus of Tyr at the expense of Phe-derived products such as anthocyanins. Furthermore, betalain-producing, ADHα-containing core Caryophyllales species accumulated more Tyr than plants not possessing ADHα (FIG. 11C). The involvement of other factors such as transcriptional regulation of betalain, anthocyanin, and Tyr/Phe pathway genes remain to be examined (Hatlestad et al., 2015; Ambawat et al, 2013), however our data provide a fascinating insight into the contribution of Tyr biosynthesis regulation to the evolution of a novel betalain pigment biosynthesis.

Prior heterologous reconstructions of specialized metabolic pathways resulted in significant accumulations of Tyr-derived plant natural products, such as a cyanogenic glycoside, dhurrin, in Arabidopsis (˜4% per dry weight, Tattersall et al., 2001; Kristensen et al., 2005) and betalains in tobacco (330 mg kg⁻¹ approaching red beet extract of 760 mg kg⁻¹, Polturak et al., 2016). In other cases, however, DODA and CYP76AD1 expression in Arabidopsis still required feeding of Tyr for betalain production (Harris et al., 2012; Sunnadeniya et al., 2016). Therefore, “pulling” a precursor (e.g. Tyr) may not be always enough to efficiently produce its downstream product, and “pushing” the precursor supply may be also important. Indeed, in red beets, increased Tyr levels have a strong positive correlation with enhanced accumulation of betalains (Wang et al., 2017), suggesting that elevated production of Tyr plays important role in overall production of betalains. Over 100-fold increase in Tyr accumulation observed in N. benthamiana leaves expressing ADHα (FIG. 10) further demonstrates an exciting opportunity to introduce Caryophyllales ADHα enzymes into other plants and microbes, deregulate Tyr biosynthesis, and boost the availability of Tyr and the production of Tyr-derived products (e.g., vitamin E, isoquinoline alkaloids including morphine).

### Additional Materials and Methods

ADH Activity from Plant Tissue Extracts

Spinach oleracea seeds (HighMowing, Wolcott, VT) and pink Dianthus barbatus (BloomIQ, Lansing, MI) seedlings were purchased from a nursery and were grown together with Arabidopsis thaliana (ecotype Columbia) in 22° C., 60% humidity, and 12/12 h light cycle growth chamber. Leaves of spinach and Arabidopsis seedlings were harvested at 3-week-old, and Dianthus barbatus leaves were harvested at 6-week-old. The crude extracts of Arabidopsis or Dianthus barbatus were prepared from ˜1 g leaf tissues according to Aryal et al. (2014). For spinach, ˜10 g leaf tissues were used to isolate the plastids according to Aryal et al. (2014) in order to avoid the undesired cytosolic polyphenol oxidase activity. Crude or plastid fractions were desalted by Sephadex G50 column to obtain protein extracts, and protein concentration of all biological replicates were adjusted to 0.06, 0.85, and 0.6 mg/mL for spinach, Dianthus barbatus, and Arabidopsis extracts, respectively. Time course ADH activity assays at 0, 1, 2, and 3 hr were performed in the presence and absence of 500 μM Tyr analog, 3-fluoro-Tyr, in 10 μL reaction containing 50 mM sodium phosphate (pH 8.0), 1 mM arogenate, 1 mM NADP⁺, 10 μg/mL tetracycline (to inhibit prokaryotic-type protein synthesis of plastids or bacterial contamination), and 0.3, 4.25, and 3 g of spinach, Dianthus, and Arabidopsis protein, respectively. The reaction was stopped by adding 20 μL methanol containing 10 μM norvaline as an internal standard. Respective boiled protein extracts were used as negative controls. ADH activity was quantified by the formation of tyrosine according to (Schenck et al., 2015), except that tyrosine was detected as o-phthalaldehyde derivative with excitation/emission wavelength of 360/455 nm by fluorescence detector, and o-phthalaldehyde derivative of the norvaline internal standard was quantified at 336 nm by DAD detector.

Analysis of Tyr Contents from Caryophyllales Tissues

Metabolite extracts of thirteen Caryophyllales species were prepared from ˜70 mg of youngest leaves, except for flowers of a Cactaceae species to avoid succulent tissues. All plants were grown and harvested at Botany Greenhouse of the University of Wisconsin-Madison. Young leaf tissues of ˜4 weeks-old Arabidopsis Columbia ecotype were used as a control. Harvested tissues were extracted by adding 400 μL extraction buffer containing methanol:chloroform (2:1, v/v) and 100 μM 4-chlorobenzoic acid (an internal standard). After adding 300 μL water and 125 μL chloroform, the mixture was vigorously mixed by a vortex mixer for 5 min and centrifuged at 20,000 g for 5 min for phase separation. The upper polar phase of 400 μL was transferred to a new centrifuge tube and dried down in a benchtop speed vacuum (Labconco, Kansas City, MO, USA). The dried polar phase was resuspended in 200 μL methanol. After centrifugation at 20,000 g for 5 min, 20 μL was injected into the Agilent 1260 HPLC equipped with Atlantis T3 C-18 column (3 μm, 2.1×150 mm, Waters, Milford, MA), and separated by the following gradient of acetonitrile (B) in 0.1% formic acid (A): 1% B for the first 5 min, followed by a linear increase to 76% B at 10 min, an isocratic elution at 76% B until 16 min, followed by re-equilibration at 1% B. Tyr was monitored with the fluorescence detector at 274 and 303 nm for excitation and emission, respectively. The internal standard was monitored by photodiode array detector at 270 nm. Statistical analyses were conducted by the Statistica Analysis Software (SAS) based on the “mixed” effect model (Pinheiro, 2000) to compare between the two groups having and not-having ADHα and using the “fixed” effect model (Milliken, 2009) to compare individual samples against Arabidopsis control.

### Reverse Transcription PCR (RT-PCR) Analysis

RT-PCR was carried out on five biological replicates for each infiltrated vector (FIG. 9B). Two technical replicates were additionally analyzed for one sample each for BvADHα and BvADHβ infiltrations. RNA was extracted and DNAse treated using the RNeasy Plant Mini Kit and the RNAse-free DNAse set (Qiagen, Hilden, Germany). cDNA was prepared using BioScript Reverse Transcriptase (Bioline Reagents, London, UK) and an oligo(dT)₁₈ primer according to the manufacturer's recommendations. A control with no reverse transcription was included to test the presence of genomic DNA. RT-PCR was performed on a 1:10 cDNA dilution with the KAPA 2G Fast DNA Polymerase kit (KAPA Biosystems, Wilmington, MA, USA) and an Eppendorf Mastercycler Nexus (Eppendorf, Hamburg, Germany). Amplification conditions were as follow: initial step of 1 min at 95° C. followed by 30 cycles of 10 s at 95° C., 10 s at 60° C. and 2 s at 72° C., and a final step of 5 min at 72° C. Amplicons were visualised on 2% agarose gel electrophoresis using ethidium bromide (0.1 μg/ml) and run at 120V for 20 min. The expected size for the reactions is 140, 90 and 111 bp for BvADHα, BvADHβ, and tGFP, respectively. Primers used are described in Table 1.

Quantitative Real-Time PCR (qRT-PCR) Analysis

For quantification of endogenous expression of BvACTIN (internal control), BvADHα, BvADH #, BvDODA, BvMYB1 and BvCYP76AD1, red beet (W357B) and sugar beet (Big Buck) plants were grown in 22° C., 60% humidity, and 12/12 hr light cycle in a growth chamber. The seedlings were harvested at 7-days after germination and the tissue was divided into cotyledon and hypocotyl. RNA was extracted (Onate-Sinchez and Vicente-Carbajosa, 2008) and DNAse treated (Ambion, Austin TX, USA) following by cDNA preparation using MLV Reverse Transcriptase (Promega, Madison, WI, USA). qRT-PCR was performed using the GoTaq qPCR Master Mix (Promega, Madison, WI, USA), and the Stratagene Mx3000P qPCR System (Agilent Technologies, Stratagene, La Jolla, CA, USA). Amplification conditions were as follow: an initial step of 1 min at 95° C. followed by 45 cycles of 15 s at 95° C., 30 s at 60° C. and 30 s at 72° C. The gene expression of BvADH was normalized using BvACTIN as an internal control and analyzed by using the relative expression of the genes. The results are shown in % expression relative to the highest sample (FIG. 1D). Primers used in all qPCR analysis are listed in Table 1.

### Phylogenetic Analysis

Amino acids from genomes (full open reading frame) and transcriptomes (full or partial open reading frame) of Brockington et al. (2015) were used in this analysis with minor modifications in species included (Table 2). The final taxon sampling in this study consisted of 95 species, with 91 ingroup species (89 transcriptomes and 2 genomes) representing 26 of the 39 families in Caryophyllales (Hernández-Ledesma et al., 2015) and four outgroup genomes from eudicots and monocots (Table 2). Amino acid sequences of the 11 functionally characterized ADH genes were used as baits to search against each of the 95 species. To maximize the sensitivity of homology searches in order to identify short and incomplete sequences from de novo assembled transcriptomes, we used SWIPE v2.0.11 (Rognes, 2011) with a high E-value cutoff of 10 and low minimal bitscore cutoff of 30. Hits from all 11 query sequences against each species were ranked from high to low by bitscore, and the top 10 hits from each species were pooled and used for the initial phylogenetic analysis.

The pooled top hits from each of the 95 species, together with the 11 baits were used as the starting sequence file (948 sequences). An initial phylogenetic analysis was conducted using MAFFT v7.215 with “--genafpair--maxiterate 1000” (Katoh & Standley, 2013). Columns with more than 90% missing data in the resulting alignment were trimmed using Phyutility v2.2.6 with “-clean 0.1” (Smith & Dunn, 2008) and a phylogeny was estimated using RAxML v8.1.5 with the model “PROTCATWAG” (Stamatakis, 2014). After visually examining the alignment and tree, tips with branch lengths that were outliers were removed (any terminal branches that had on average more than two substitutions for each amino acid site; or more than ten times longer than its sister group and on average had more than one substitution per site; Yang and Smith, 2014). Monophyletic or paraphyletic tips that belonged to the same species from transcriptome data most often resulted from isoforms produced during de novo assembly. These were masked, leaving only the tip with the highest number of aligned characters (Yang and Smith, 2014). Internal branches with molecular branch lengths longer than 1 were likely due to distantly related paralogs or assembly artifacts and were pruned. A large number of distantly related genes, isoforms, and assembly errors were removed during the tip trimming and long branch removing process, with 251 sequences left. A new fasta file was written from remaining tips, and this alignment, tree building, and tree trimming procedure was repeated once, with 229 sequences left. Following the homology search and filtering, we extracted the Caryophyllales ADH gene lineage rooted by outgroup genomes (Yang and Smith, 2014). While visually examining alignment and tree we found the sequence Cham@c36044_g1_i2_242_1480_minus that belonged to Chenopodium giganteum, but were placed in between ADHα and ADHβ, outside of Chenopodiaceae. Further examination of the alignment showed that the half of the sequence was closely related to ADHα, and the other half closely related to ADHβ. Although this can be real, it is most likely an assembly error and was removed from the analysis. Indeed, Chenopodium giganteum had additional, correctly assembled ADHα and ADHO copies nested in respective Chenopodiaceae clades. Therefore this putative chimeric sequence was removed.

Remaining sequences belonged to the Caryophyllales ADH lineage were aligned with MAFFT with “--genafpair --maxiterate 1000” and trimmed by Phyutility with “-clean 0.3”. An alternative alignment was constructed with PRANK v140603 using default settings (Loytynoja & Goldman, 2008; 2010), poorly aligned sequences were manually removed, and trimmed by Phyutility with “-clean 0.1”. We used two alternative alignment methods because MAFFT tends to force regions to align even when they are highly divergent whereas PRANK tends to introduce lots of gaps in highly divergent regions. On the other hand, PRANK is an iterative alignment, tree building, and refinement pipeline that we run five iterations before obtaining the final alignment. For both trimmed alignments, a phylogenetic tree was constructed using RAxML with “-m PROTCATAUTO” and 200 rapid bootstrap replicates to evaluate support. Given that the resulting tree topologies and support values using both alignments were very similar we are presenting the results from MAFFT. The code used in the phylogenetic analysis is available from bitbucket.org/yangya/adh_2016.

### Testing for Relaxed Section in Caryophyllaceae

To test for shift in selection pressure in ADHα associated with loss of betalain, we carried out selection analysis on a reduced data set that included representative sequences across ADHα that were either verified by Sanger sequencing or by mapping reads back to the de novo assembled contigs and carefully examining read coverages visually.

Within the family Caryophyllaceae, ADHα expression was detected in the transcriptome of only the subfamily Paronychioideae. Those ADHα transcripts from Corrigiola litoralis and Telephium imperati were both confirmed by PCR and Sanger sequencing. Two Spergularia media fragments from transcriptome assembly were both belonged to ADHα and are non-overlapping in the alignment. These two fragments could be from two loci or from a single locus. To distinguish between these two scenarios, we first extended the two fragments separately using Assembly by Reduced Complexity (Hunter et al., 2015, ARC v.1.1.3) with maximum 10 cycles, Bowtie 2 v2.2.8 (Langmead & Salzberg, 2012) for read mapping and Newbler v2.9 (454 Life Sciences, downloaded Mar. 17, 2015) for assembly. After extending the original assembly and aligning it with other ADHα sequences, the two extended fragments were still 22 base pairs apart. To evaluate whether these two fragments were supported by raw reads we concatenated the two fragments by fixing the direction and adding 22 Ns to the middle, and mapped raw reads to the concatenated reference using Bowtie 2 with the setting “--phred64--very-fast-local”. The 22 bp gap was highly supported by read pairs and the joined read were kept for subsequent dN/dS analysis. We carried out the same procedure for Polycarpaea repens but were unable to join the reads nor confirm they are paralogs due to low read coverage and a longer gap between the two fragments. Therefore, the two fragments were kept in the alignments for phylogenetic analysis but were removed for dN/dS analysis.

To obtain ADHα sequences from additional species of Caryophyllaceae, primers were designed to the conserved portion of the Spergularia media contig, and were used to amplify ADHα sequences from the closely related Spergularia marina. Inverse PCR was used to obtain ADHα sequences from Spergularia marina, Paronychia polygonfolia and Herniaria latifolia. For inverse PCR, genomic DNA was digested with restriction enzymes EcoRI and MfeI, and fragments were circularised with T4 ligase (Biolabs, New England). Nested primers were used to amplify the fragment containing the ADHα ortholog. Amplified products were sanger sequenced to acquire the 5′ and 3′ terminals of the locus. In summary, a total of six well-supported ADHα sequences were then taken forward for the dN/dS selection analyses.

Our final alignment for selection analysis included eight ADHα sequences in Caryophyllaceae and six additional sequences from representative betalain-producing species across rest of the ADHα lineage. We first trimmed the alignment to remove signal peptide and poorly aligned ends, leaving the region from BvADHα amino acid no. 79 to 354 that covered the enzyme active domain. We then carried out phylogenetic analyses for both alignments in RAxML, with the model “GTRCAT” for the codon alignment and “PROTCATAUTO” for the amino acids alignment, and 200 rapid bootstrap replicates to evaluate node support (FIG. 14A, 14B). To quantify the rate shift, we carried out RELAX analysis (Wertheim et al., 2014) as implemented in the online portal Datamonkey (Kosakovsky Pond & Frost, 2005, accessed Mar. 19, 2016), using the trimmed CDS matrix with Polycarpaea repens removed. RELAX has the advantage of distinguishing between increased positive selection vs. reduced purifying selection, both of which would result in accelerated average dN/dS values. We designated all crown branches in Caryophyllaceae as the testing branches and the rest branches as the background. We fitted the partitioned MG94xREV model that assumes all sites having unified dN and dS value, allowing the rate to vary between the test and background branches. We also fitted the RELAX model that takes site heterogeneity into account. The RELAX null model assumes all background and test branches share the same rate in each rate category, whereas the RELAX alternative model allows substitution rate to vary between the test and background branches in each rate category, and sites can move among rate categories.

### Example 2: Overexpression of BvADHα but not BvADHβ Leads to High Accumulation of Tyrosine in Arabidopsis thaliana

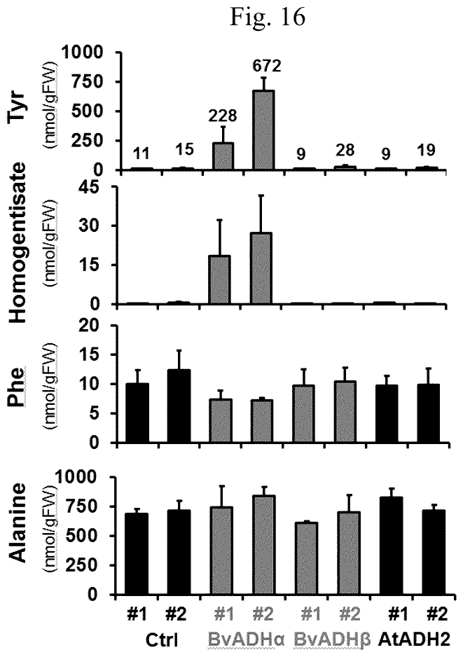

Beta vulgaris accumulates high amounts of endogenous tyrosine as well as its derived metabolites betalains due to the presence of the tyrosine-insensitive BvADHα enzyme. To further test if the lack of BvADHα feedback regulation is a critical factor for high tyrosine accumulation in plant tissues, BvADHα, BvADHβ, and Arabidopsis ADH2 (AtADH2) were individually overexpressed by the 35S promoter of the cauliflower mosaic virus (CaMV) in A. thaliana Col-0 background. The empty vector containing no gene was also introduced as a negative control. Gas chromatography-mass spectrometry (GC-MS) based metabolite analysis showed that overexpression of BvADHα but not BvADHβ or AtADH2 leads to much higher accumulation of tyrosine than the empty vector control (nearly 50-fold increase, FIG. 16). In addition, BvADHα expression resulted in a slightly reduction of an aromatic amino acid phenylalanine and drastic increase in homogentisate, the downstream product of tyrosine and precursor of tocopherols (vitamin E). No differences were observed for most amino acids, including alanine. These results provide proof-of-concept demonstration that the production of tyrosine can be substantially enhanced by the expression of a tyrosine-insensitive ADH enzyme (i.e. BvADHα) in plant tissues. In addition, the observed increase of homogentisate as a consequence of high levels of tyrosine suggests that Tyr availability is a limit-step for the production of Tyr-derived secondary metabolites in plants such as tocopherols or betalains.

### Material and Methods

Cloning of BvADHα, BvADHβ and AtADH2 cDNAs into Overexpression Binary Vector

Total RNA isolated from Beta vulgaris and Arabidopsis thaliana leaf tissues were used to synthesize cDNA using random primers and the High Capacity cDNA Reverse Transcription Kit (Applied Biosystems). Specific oligonucleotides to amplify each of the desired cDNAs were designed using In-Fusion® Primer design tool (Clontech). PCR fragments were obtained using Phusion High-Fidelity DNA polymerase and cloned into the binary vector DF_264 vector, downstream of the 35S CaMV promoter, using the In-Fusion® HD cloning kit. Plasmid was linearized with the restriction enzymes XbaI and BamHI (FastDigest, Thermo Scientific) and the enzymes sites were preserved after cloning. XbaI site is upstream of ATG start codon and BamHI is downstream of TAA stop codon. All reactions were performed accordingly with the instructions of the manufacter. In-Fusion cloning reactions were transformed into E. coli Stellar™ Competent cells (Clontech) and positive colonies were selected on LB agar plates containing 50 μg/mL Spectinomycin. Antibiotic resistance colonies were confirmed for the presence of the cDNA insert by colony PCR and submitted to plasmid isolation. cDNA inserts were checked for possible point mutations by SANGER sequencing the obtained plasmids using primers annealing at the 35S CaMV promoter and NOS terminator. Confirmed vectors were transformed into Agrobacterium tumefaciens GV3101 by freeze-thaw method.

### Arabidopsis Transformation and Transgenic Selection.

Flowering A. thaliana Col-0, 5-6 weeks old, were used to plant transformation by floral dip (Bent A (2006) Arabidopsis thaliana floral dip transformation method. Methods Mol Biol. 343: 87-103). Briefly, flower buds were submerged into Agrobacterium GV3101 solution. The excess of solution was removed using absorbent paper. Plants were transfer to a close container to preserve humidity and kept in a dark environment for 16 hours after transformation. After this period of time, plants were acclimated back to the growth chamber. The transformation process was repeated after 5 days of the first transformation and plants were kept in the growth chamber until harvesting. T₀ seeds were chlorine sterilized and germinated on ½ Force Murashige and Skoog (MS) agar plates supplemented with 1% Sucrose and 100 μg/mL of Gentamycin. 10 positive T₁ seedlings for each construct were transferred to soil and seeds were harvested for each individual plant. Transgenic lines were then checked for the number of insertions based on the segregation ratio of antibiotic resistant T₂ seedlings. Single-insertion homozygous T₂ lines were then germinated on soil and 4-weeks old plants were analyzed for Tyr and other organic acids contents by gas chromatography-mass spectrometry analysis (GC-MS).

### GC-MS Analysis

Four-week old Arabidopsis plants overexpressing BvADHα, BvADHβ, AtADH2 or empty vector were submitted to GC-MS analysis. Briefly, approximately 30 mg of fresh leaf tissue was excised from at least 3 plants of each transgenic line to compound one biological replicate. Tissue sample was transferred to a 1.5 mL microcentrifuge tube and 400 μL of solvent extraction solution [Methanol:Chloroform (2:1) with 100 μM norvaline]. Three 3 mm glass beads were added to each tube and samples were submitted to GenoGrindr (1500 strokes/min) for 5 min. After a brief spin 300 μL of water, followed by 125 μL of Chloroform were added to each sample. Samples were vortex on high for 30 seconds and centrifuged at 21000×g for 5 minutes to achieve phase separation. The aqueous phase was carefully transferred to a new 1.5 mL tube and transfer to speedvac system at room temperature until completely dry. After dry, the polar phase compounds were resuspended in 210 μL of methanol containing 100 μM 4-chlorobenzoic acid. Samples were sonicated for 10 min and insoluble remaining debris was removed by centrifugation at 21000×g for 5 min. at room temperature. 100 μL of supernatant was transferred into a glass vial and the methanol was dry out in the speed vac. After dry, the inserts were transferred to a glass vial and the pellets were ressuspended in 40 μL pyridine. Samples were submitted to sonication for 10 min and 40 μL of N-methyl-N-(tert-butyldimethylsilyl)trifluoroacetamide with 1% tertbutyldimetheylchlorosilane (MTBSTFA+1% t-BDMCS) was added to each sample. Samples were incubated at 80° C. for 1 hour and transferred to analysis on GC-MS. The GC-MS was stablished as Hold at 70° C. for 2 min, increased to 250° C. by 5° C. per min., then hold at 300° C. for 10 min. Amino acid standard (Sigma, #AAS18) was used to stablish the standard curve of each amino acid. Peak areas were normalized by the internal standard norvaline and by fresh tissue weight (g).

### Example 3—in Planta Expression of Tyr-Insensitive BvADHα Leads to Enhanced Accumulation of Tyr in Arabidopsis

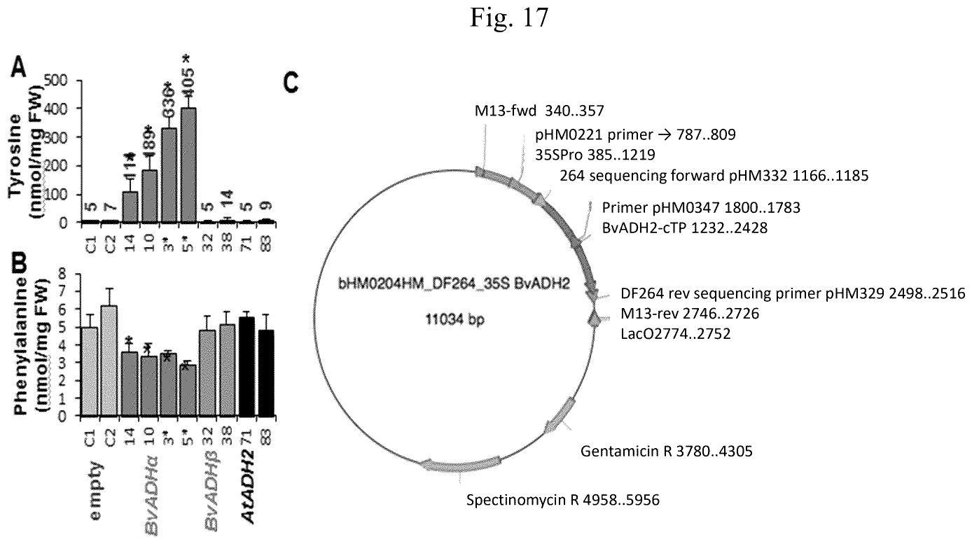

BvADHα was heterologously expressed in Arabidopsis, which only has Tyr-inhibited ADH enzymes (Rippert and Matringe, 2002a; Rippert and Matringe, 2002b; Schenck et al., 2015). Overexpression of BvADHα, but not Tyr-inhibited BvADHβ or AtADH2, resulted in elevated Tyr accumulation by up to 60-fold compared to empty vector controls in T3 single insertion homozygous lines (FIG. 17). Also, the BvADHα lines reduced levels of Phe. Thus, expression of de-regulated BvADHα can increase the carbon flow through the shikimate pathway and direct away from Phe biosynthesis to drastically enhance availability of Tyr.

### Example 4—Heterologous Expression of Tyr-Insensitive BvADHα Leads to Hyper-Accumulation of Tyr in Glycine max (Soybean)

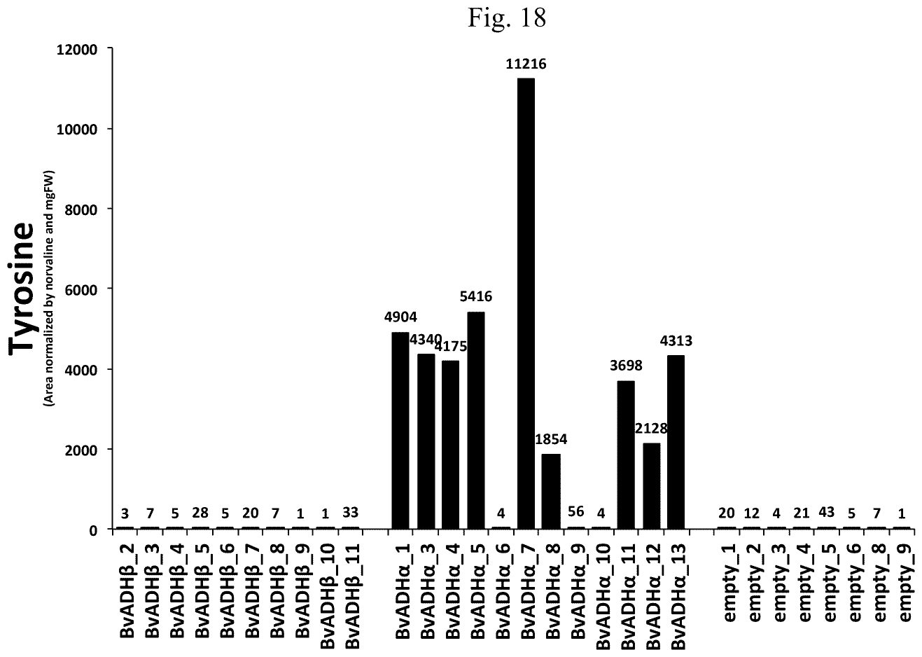

BvADHα or BvADHβ was also heterologously expressed in Glycine max (soybean), which has both Tyr-inhibited ADH and Tyr-insensitive PDH enzymes (Schenck et al., 2015). When Tyr levels were analyzed in the leaves of antibiotic resisitant T₁ transgenic lines, nine out of twelve BvADHα overexpression lines showed nearly 1,000 fold increase in Tyr relative to empty vector control (FIG. 18). All of BvADHβ transgenic lines showed basal levels of Tyr similar to empty vector controls. Three BvADHα lines with low Tyr were likely unsuccessful transformants.

### REFERENCES

Ambawat S, Sharma P, Yadav N R, Yadav R C. 2013. MYB transcription factor genes as regulators for plant responses: an overview. Phyisyiology and Molecular Biology of Plants 19: 307-321.
Bate-Smith E C. 1962. The phenolic constituents of plants and their taxonomic significance. Botanical Journal of the Linnean Society 58: 95-173.
Barshandy H, Jalkanen S, Teeri T H. 2015. Within leaf variation is the largest source of variation in agroinfiltration of Nicotiana benthamiana. Plant Methods 11: 47.
Beaudoin G A W, Facchini P J. 2014. Benzylisoquinoline alkaloid biosynthesis in opium poppy. Planta 240: 19-32.
Bentley R. 1990. The ahikimate pathway—A metabolic tree with many branche. Critical Reviews in Biochemistry and Molecular Biology 25: 307-84.
Biancardi E, Panella L W, and Lewellen R. 2012. Beta maritima: The origin of beets. New York: Springer.
Bonvin J, Aponte R A, Marcantonio M, Singh S, Christendat D, Turnbull J L. 2006. Biochemical characterization of prephenate dehydrogenase from the hyperthermophilic bacterium Aquifex aeolicus. Protein Science 15: 1417-32.
Brockington S F, Walker R H, Glover B J, Soltis P S, Soltis D E. 2011. Complex pigment evolution in the Caryophyllales. New Phytologist 190: 854-864.
Brockington S F, Yang Y, Gandia-Herrero F, Covshoff S, Hibberd J M, Sage R F, Wong G K S, Moore M J, Smith S A. 2015. Lineage-specific gene radiations underlie the evolution of novel betalain pigmentation in Caryophyllales. New Phytologist 207: 1170-1180.
Byng G, Whitaker R, Elick C, Jensen ROYA. 1981. Enzymology of L-tyrosine biosynthesis in corn (Zea mays). Phytochemistry 20: 1289-1292.
Chen F, Tholl D, Bohlmann J, Pichersky E. 2011. The family of terpene synthases in plants: A mid-size family of genes for specialized metabolism that is highly diversified throughout the kingdom. Plant Journal 66: 212-229.
Christinet L, Burdet F, Zaiko M, Hinz U, Zrÿd J P. 2004. Characterization and functional identification of a novel plant 4,5-extradiol dioxygenase involved in betalain pigment biosynthesis in Portulaca grandiflora. Plant Physiology 134: 265-274.
Clement J S, Mabry T J. 1996. Pigment evolution in the caryophyllales: A systematic overview. Botanica Acta 109: 360-367.
Connelly J A and, Conn E E. 1986. Tyrosine biosynthesis in Sorghum bicolor: isolation and regulatory properties of arogenate dehydrogenase. Zeitschrift für Naturforschung C 41: 69-78.
Dal Cin V, Tieman D M, Tohge T, McQuinn R, de Vos RCH, Osorio S, Schmelz E, Taylor M G, Smits-Kroon M T, Schuurink R C, et al. 2011. Identification of genes in the phenylalanine metabolic pathway by ectopic expression of a MYB transcription factor in tomato fruit. The Plant Cell 23: 2738-2753.
Delaux P M, Varala K, Edger P P, Coruzzi G M, Pires J C, Ané J M. 2014. Comparative phylogenomics uncovers the impact of symbiotic associations on host genome evolution. PLoS Genetics 10: e1004487.
Brockington S F, Alexandre R, Ramdial J, Moore M J, Crawley S, Dhingra A, Hilu K, Soltis P S. 2009. Phylogeny of the Caryophyllales sensu lato: Revisiting hypotheses on pollination biology and perianth differentiation in the core Caryophyllales. International Journal of Plant Sciences 170: 627-643.
Dohm J C, Minoche A E, Holtgrawe D, Capella-Gutierrez S, Zakrzewski F, Tafer H, Rupp O, Sörensen T R, Stracke R, Reinhardt R, et al. 2014. The genome of the recently domesticated crop plant sugar beet (Beta vulgaris). Nature 505: 546-549.
Dornfeld C, Weisberg A J, C R K, Dudareva N, Jelesko J G, Maeda H A. 2014. Phylobiochemical characterization of class-lb aspartate/prephenate aminotransferases reveals evolution of the plant arogenate phenylalanine pathway. The Plant Cell 26: 3101-3114.
Engler C, Youles M, Gruetzner R, Ehnert T M, Werner S, Jones J D G, Patron N J, and Marillonnet S. 2014. A golden gate modular cloning toolbox for plants. ACS Synthetic Biology 3: 839-843.
Gaines, C. G., Byng G S, Whitaker R J and, Jensen R A. 1982. L-Tyrosine regulation and biosynthesis via arogenate dehydrogenase in suspension-cultured cells of Nicotiana silvestris Speg. et Comes. Planta 156: 233-240.
Gandia-Herrero F, Garcia-Carmona F. 2012. Characterization of recombinant Beta vulgaris 4,5-DOPA-extradiol-dioxygenase active in the biosynthesis of betalains. Planta 236: 91-100.
Gleadow R M, Møller B L. 2014. Cyanogenic glycosides: synthesis, physiology, and phenotypic plasticity. Annual Review of Plant Biology 65: 155-185.
Greenberg A K and Donoghue M J. 2011. Molecular systematics and character evolution in Caryophyllaceae. TAXON60: 1637-1652
Goldman I L. 1996. A list of germplasm releases from the University of Wisconsin table beet breeding program. HortScience 31: 880-881.
Hanson M et al. 1996. Evolution of anthocyanin biosynthesis in maize kernels: the role of regulatory and enzymatic loci. Genetics 143: 1395-1407.
Hatlestad G J, Sunnadeniya R M, Akhavan N, Gonzalez A, Goldman I L, McGrath J M, Lloyd A M. 2012. The beet R locus encodes a new cytochrome P450 required for red betalain production. Nature Genetics 44: 816-820.
Hudson, G S, Wong, V., and Davidson B. 1984. Chorismate mutase/prephenate dehydrogenase from Escherichia coli K12: purification, characterization, and identification of a reactive cysteine. Biochemistry 23: 6240-6249.
Ibarra-Laclette E, Zamudio-Hernandez F, Pérez-Torres C A, Albert V A, Ramirez-Chávez E, Molina-Torres J, Fernandez-Cortes A, Calderón-Vázquez C, Olivares-Romero J L,
Herrera-Estrella A, et al. 2015. De novo sequencing and analysis of Lophophora williamsii transcriptome, and searching for putative genes involved in mescaline biosynthesis. BMC Genomics 16: 657.
Khan M I. 2015. Plant betalains: safety, antioxidant activity, clinical efficacy, and bioavailability. Comprehensive Reviews in Food Science and Food Safety 15: 316-330.
Kajikawa M, Sierro N, Kawaguchi H, BakaherN, Ivanov N V, Hashimotp T, Shoji T. 2017. Genomic insights into the evolution of the nicotine biosynthesis pathway in tobacco. Plant Physiology 4: 999-1011.
Kristensen C, Morant M, Olsen C E, Ekstrom C T, Galbraith D W, Moller B L, Bak S. 2005. Metabolic engineering of dhurrin in transgenic Arabidopsis plants with marginal inadvertent effects on the metabolome and transcriptome. Proceedings of the National Academy of Sciences of the United States of America 102: 1779-1784.
Lee, E. J., An, D., Nguyen, C. T. T. Lee, E. J., An, D. Nguyen, C. T., Patill, B. S., Kim, J., & Yoo K. 2014. Betalain and betaine composition of greenhouse or field produced beetroot (Beta vulgaris L.) and inhibition of HepG2 cell proliferation. Journal of Agriculture and Food Chemistry 62: 1324-1331.
Legrand P, Dumas R, Seux M, Rippert P, Ravelli R, Ferrer J L, Matringe M. 2006. Biochemical characterization and crystal structure of Synechocystis arogenate dehydrogenase provide insights into catalytic reaction. Structure 14: 767-776.
Mabry T J. 1964. The betacyanins, a new class of red violet pigments, and their phylogenetic significance. In: Leone C A, ed. Taxonomic biochemistry, physiology, and serology. New York, N Y, USA: Ronald Press: 239-254.
Maeda H, Dudareva N. 2012. The shikimate pathway and aromatic amino acid biosynthesis in plants. Annual Review of Plant Biology 63: 73-105.
Mene-Saffrane L, Jones A D, DellaPenna D. 2010. Plastochromanol-8 and tocopherols are essential lipid-soluble antioxidants during seed desiccation and quiescence in Arabidopsis. Proceedings of the National Academy of Sciences of the United States of America 107: 17815-17820.
Millgate A G, Pogson B J, Wilson I W, Kutchan™, Zenk M H, Gerlach W L, Fist A J, Larkin P J. 2004. Analgesia: Morphine-pathway block in top1 poppies. Nature 431: 413-414.
Mizutani M, Ohta D. 2010. Diversification of P450 genes during land plant evolution. Annual Review of Plant Biology 61: 291-315.
Moghe G D, Last R L. 2015. Something old, something new: Conserved enzymes and the evolution of novelty in plant specialized metabolism. Plant Physiology 169: 1512-23.
Neelwarne B, & Halagur S B. 2012. Red beet: an overview. In B. Neelwarne (Ed.), Red Beet Biotechnology: Food and Pharmaceutical Applications. New York, New York USA, Springer: 1-43.
Panchy N, Lehti-Shiu M, Shiu S-H. 2016. Evolution of gene duplication in plants. Plant Physiology 171: 2294-2316.
Pencharz P B, Hsu J W-C, Ball R O. 2007. Aromatic amino acid requirements in healthy human subjects. The Journal of Nutrition 137: 1576S-1578S.
Pichersky E, Lewinsohn E. 2011. Convergent evolution in plant specialized metabolism. Annual Review of Plant Biology 62: 549-566.
Polturak G, Breitel D, Grossman N, Sarrion-Perdigones A, Weithorn E, Pliner M, Orzaez D, Granell A, Rogachev I, Aharoni A. 2016. Elucidation of the first committed step in betalain biosynthesis enables the heterologous engineering of betalain pigments in plants. New Phytologist 210: 269-283.
Rapp R A, Haigler C H, Flagel L, Hovav R H, Udall J A, Wendel J F. 2010. Gene expression in developing fibres of upland cotton (Gossypium hirsutum L.) was massively altered by domestication. BMC Biology 8: 1-15.
Rippert P, Matringe M. 2002a. Purification and kinetic analysis of the two recombinant arogenate dehydrogenase isoforms of Arabidopsis thaliana. European Journal of Biochemistry 269: 4753-4761.
Rippert P, Matringe M. 2002b. Molecular and biochemical characterization of an Arabidopsis thaliana arogenate dehydrogenase with two. Plant Molecular Biology 48: 361-368.
Rippert P, Puyaubert J, Grisollet D, Derrier L, Matringe M. 2009. Tyrosine and phenylalanine are synthesized within the plastids in Arabidopsis. Plant Physiology 149: 1251-1260.
Rong J, Lammers Y, Strasburg J L, Schidlo N S, Ariyurek Y, de Jong T J, Klinkhamer P G L, Smulders M J M, Vrieling K. 2014. New insights into domestication of carrot from root transcriptome analyses. BMC Genomics 15: 895.
Rubin J L, Jensen R a. 1979. Enzymology of L-tyrosine biosynthesis in mung bean (Vigna radiata [L.] Wilczek). Plant Physiology 64: 727-734.
Schenck C A, Holland C K, Schneider M R, Men Y, Lee S G, Jez J M & Maeda H A. 2017. Molecular Basis of the evolution of alternative tyrosine biosynthetic routes in plants. Nature Chemocal Biology 13: 1029-1035.
Schenck C A, Chen S, Siehl D L, Maeda H A. 2015. Non-plastidic, tyrosine-insensitive prephenate dehydrogenases from legumes. Nature Chemical Biology 11: 52-57.
Shimada S, Inoue Y T, Sakuta M. 2005. Anthocyanidin synthase in non-anthocyanin-producing Caryophyllales species. The Plant Journal 44: 950-959.
Siehl D L. 1999. The biosynthesis of tryptophan, tyrosine, and phenylalanine from chorismate in Plant Amino Acids: Biochemistry and Biotechnology. Singh B, ed. New York: CRC Press, New York, 171-204.
Sparkes I A, Runions J, Kearns A, and Hawes C. 2006. Rapid, transient expression of fluorescent fusion proteins in tobacco plants and generation of stably transformed plants. Nature Protocols 1: 2019-2025.
Sun W, Shahinas D, Bonvin J, Hou W, Kimber M S, Turnbull J, Christendat D. 2009. The crystal structure of Aquifex aeolicus prephenate dehydrogenase reveals the mode of tyrosine inhibition. Journal of Biological Chemistry 284: 13223-13232.
Sunnadeniya R, Bean A, Brown M, Akhavan N, Hatlestad G, Gonzalez A, Symonds V V, Lloyd A. 2016. Tyrosine hydroxylation in betalain pigment biosynthesis is performed by cytochrome P450 enzymes in beets (Beta vulgaris). PLoS ONE 11: 1-16.
Tanaka Y, Sasaki N, Ohmiya A. 2008. Biosynthesis of plant pigments: anthocyanins, betalains and carotenoids. Plant Journal 54: 733-749.
Tattersall D B, Bak S, Jones P R, Olsen C E, Nielsen J K, Hansen M L, Høj P B, Møller B L. 2001. Resistance to an herbivore through engineered cyanogenic glucoside synthesis. Science 293: 1826-1828.
Tzin, V. & Galili G. 2010. The biosynthetic pathways for shikimate and aromatic amino acids in Arabidopsis thaliana. The Arabidopsis Book/American Society of Plant Biologists 8: e0132.
Wang M, Lopez-Nieves S, Goldman I L, and Maeda H A. 2017. Limited tyrosine utilization explains lower betalain contents in yellow than red table beet genotypes. Journal of Agricultural and Food Chemistry 65: 4305-4313.
Wang X, Xiao H, Chen G, Zhao X, Huang C, Chen C, Wang F. 2011. Isolation of high-quality RNA from Reaumuria soongorica, a desert plant rich in secondary metabolites. Molecular Biotechnology 48: 165-172.
Weber E, Engler C, Gruetzner R, Werner S, and Marillonnet S. 2011. A modular cloning system for standardized assembly of multigene constructs. PLoS One 18: 6:e16765.
Weng J K. 2014. The evolutionary paths towards complexity: A metabolic perspective. New Phytologist 201: 1141-1149.
Weng J K, Philippe R N, Noel J P. 2012. The rise of chemodiversity in plants. Science 336; 1667-1670.
Wertheim J O, Murrell B, Smith M D, Kosakovsky Pond S L, Scheffler K. 2014. RELAX: detecting relaxed selection in a phylogenetic framework. Molecular Biology and Evolution 32: 1-13.
Xu S, Brockmöller T, Navarro-Quezada A, Kuhl H, Gase K, Ling Z, Zhou W, Kreitzer C, Stanke M, Tang H, Lyons E, Pandey P, Pandey S P, Timmermann B, Gaquerel E, Baldwin I T. 2017. Wild tobacco genomes reveal the evolution of nicotine biosynthesis. Proceedings of the National Academy of Sciences of the United States of America 114: 6133-6138.
Xu S, Huang Q, Lin C, Lin L, Zhou Q, Lin F and He E. 2016. Transcriptome comparison reveals candidate genes responsible for the betalain-/anthocyanidin-production in bougainvilleas. Functional Plant Biology 43: 278-286.
Yagi M, Kosugi S, Hirakawa H, Ohmiya A, Tanase K, Harada T, Kishimoto K, Nakayama M, Ichimura K, Onozaki T, et al. 2014. Sequence analysis of the genome of carnation (Dianthus caryophyllus L.). DNA Research 21: 231-241.
Zhang R, Guo C, Zhang W, Wang P, Li L, Duan X, Du Q, Zhao L, Shan H, Hodges S a, et al. 2013. Disruption of the petal identity gene APETALA3-3 is highly correlated with loss of petals within the buttercup family (Ranunculaceae). Proceedings of the National Academy of Sciences of the United States of America 110: 5074-9.
Aryal U K, Xiong Y, McBride Z, Kihara D, Xie J, Hall M C, Szymanski D B. 2014. A proteomic strategy for global analysis of plant protein complexes. The Plant Cell 26: 3867-3882.
Brockington S F, Yang Y, Gandia-Herrero F, Covshoff S, Hibberd J M, Sage R F, Wong G K S, Moore M J, Smith S A. 2015. Lineage-specific gene radiations underlie the evolution of novel betalain pigmentation in Caryophyllales. New Phytologist 207: 1170-1180.
Hernandez-Ledesma P, Berendsohn W G, Borsch T, Mering S V, Akhani H, Arias S, Castañeda-Noa I, Eggli U, Eriksson R, Flores-Olvera H, Fuentes-Bazán S, Kadereit G, Kak C, Korotkova N, Nyffeler R, Ocampo G, Ochoterena H, Oxelman B, Rabeler R K, Sanchez A, Schlumpberger B O & Uotila P. 2015. A taxonomic backbone for the global synthesis of species diversity in the angiosperm order Caryophyllales. Willdenowia 45: 281-383.
Hunter S S, Lyon R T, Sarver B A J, Hardwick K, Forney L J, Settles M L. 2015. Assembly by Reduced Complexity (ARC): a hybrid approach for targeted assembly of homologous sequences. bioRxiv. doi:10.1101/014662.
Katoh K, Standley D M. 2013. MAFFT multiple sequence alignment software version 7: Improvements in performance and usability. Molecular Biology and Evolution 30: 772-780.
Kosakovsky Pond S L, Frost S D W. 2005. Datamonkey: Rapid detection of selective pressure on individual sites of codon alignments. Bioinformatics 21: 2531-2533.
Langmead B, Salzberg S L. 2012. Fast gapped-read alignment with Bowtie 2. Nature Methods 9: 357-359.
Löytynoja A, Goldman N. 2008. Phylogeny-aware gap placement prevents errors in sequence alignment and evolutionary analysis. Science (New York, N. Y.) 320: 1632-1635.
Löytynoja A, Goldman N. 2010. webPRANK: a phylogeny-aware multiple sequence aligner with interactive alignment browser. BMC Bioinformatics 11: 579.
Milliken, G A. 2009. Analysis of messy data. Boca Raton:CRC Press.
Oñate-Sánchez L, and Vicente-Carbajosa J. 2008. DNA-free RNA isolation protocols for Arabidopsis thaliana, including seeds and siliques. BMC Research Note 1: 93.
Pinheiro, J C. 2000. Mixed-effects models in S and S-PLUS. New York:Springer.
Rognes T. 2011. Faster Smith-Waterman database searches with inter-sequence SIMD parallelisation. BMC bioinformatics 12: 221.
Schenck C A, Chen S, Siehl D L, Maeda H A. 2015. Non-plastidic, tyrosine-insensitive prephenate dehydrogenases from legumes. Nature Chemical Biology 11: 52-57.
Smith S A, Dunn C W. 2008. Phyutility: A phyloinformatics tool for trees, alignments and molecular data. Bioinformatics 24: 715-716.
Stamatakis A. 2014. RAxML version 8: A tool for phylogenetic analysis and post-analysis of large phylogenies. Bioinformatics 30: 1312-1313.
Wertheim J O, Murrell B, Smith M D, Kosakovsky Pond S L, Scheffler K. 2014. RELAX: Detecting relaxed selection in a phylogenetic framework. Molecular biology and evolution 32: 1-13.
Yang, Y. and S. A. Smith. 2014. Orthology inference in non-model organisms using transcriptomes and low-coverage genomes: improving accuracy and matrix occupancy for phylogenomics. Molecular Biology and Evolution 31: 3081-3092.

We claim:

1. A cDNA polynucleotide encoding a polypeptide having at least 60% sequence identity to a polypeptide selected from the group consisting of any one of SEQ ID NOS: 1-20, 43, 45, or 47 and functional fragments of any one of SEQ ID NOS: 1-20, 43, 45, or 47.

2. The cDNA polynucleotide of claim 1, wherein the cDNA polynucleotide comprises any one of SEQ ID NOS: 21-40, 44, 46, or 48.

3. The cDNA polynucleotide of claim 1, wherein the cDNA polynucleotide is codon-optimized for expression in a cell.

4. The cDNA polynucleotide of claim 3, wherein the cell is a plant cell, bacterial cell, or fungal cell.

5. The cDNA polynucleotide of any one of claims 1-4, wherein the polypeptide is not sensitive to tyrosine inhibition.

6. The cDNA of any one of claims 1-5, wherein the polypeptide maintains at least 50% of its ADH activity in the presence of 10 μM tyrosine.

7. A construct comprising a heterologous promoter operably linked to any one of the cDNA polynucleotides of claims 1-6 or a polynucleotide encoding a polypeptide having at least 80% sequence identity to a polypeptide selected from the group consisting of any one of SEQ ID NOS: 1-20, 43, 45, or 47 and functional fragments of any one of SEQ ID NOS: 1-20, 43, 45, or 47.

8. The construct of claim 7, wherein the heterologous promoter is a plant promoter.

9. The construct of any one of claims 7-8, wherein the heterologous promoter is an inducible promoter or a tissue-specific promoter.

10. A vector comprising any one of the cDNA polynucleotides of claims 1-6 or any one of the constructs of claims 7-9.

11. The vector of claim 10, wherein the vector comprises a plasmid.

12. A cell comprising any one of the cDNA polynucleotides of claims 1-6, any one of the constructs of claims 7-9, or any one of the vectors of claims 10-11.

13. The cell of claim 12, wherein the cell is a plant cell.

14. The cell of claim 13, wherein the plant cell is selected from a soybean plant cell, a mung bean plant cell, an opium poppy plant cell, a quinoa plant cell, an alfalfa plant cell, a rice plant cell, a wheat plant cell, a corn plant cell, a sorghum plant cell, a barley plant cell, a millet plant cell, an oat plant cell, a rye plant cell, a rapeseed plant cell, a beet plant cell, and a miscanthus plant cell.

15. The cell of claim 12, wherein the cell is a bacterial or fungal cell.

16. A seed comprising the any one of the cDNA polynucleotides of claims 1-6, any one of the constructs of claims 7-9, any one of the vectors of claims 10-11, or the cell of claim 12.

17. A plant grown from the seed of claim 16.

18. A plant comprising any one of the cDNA polynucleotides of claims 1-6, any one of the constructs of claims 7-9, any one of the vectors of claims 10-11, or any one of the cells of claims 12-14.

19. The plant of claim 18, wherein the plant is selected from a beet plant, a soybean plant, a mung bean plant, an opium poppy plant, a quinoa plant, an alfalfa plant, a rice plant, a wheat plant, a corn plant, a sorghum plant, a barley plant, a millet plant, an oat plant, a rye plant, a rapeseed plant, and a miscanthus plant.

20. A part, progeny, or asexual propagate of any one of the plants of claims 17-19.

21. A method for increasing production of at least one product of the tyrosine or HPP pathways in a cell comprising introducing any one of the cDNA polynucleotides of claims 1-6, any one of the constructs of claims 7-9, or any one of the vectors of claims 10-11 into the cell.

22. The method of claim 21, wherein the cell is a plant cell.

23. The method of claim 22, wherein the plant cell is selected from a soybean plant cell, a mung bean plant cell, an opium poppy plant cell, a quinoa plant cell, an alfalfa plant cell, a rice plant cell, a wheat plant cell, a corn plant cell, a sorghum plant cell, a barley plant cell, a millet plant cell, an oat plant cell, a rye plant cell, a rapeseed plant cell, a beet plant cell, and a miscanthus plant cell.

24. The method of claim 21, wherein the cell is a bacterial or fungal cell.

25. The method of any one of claims 21-24, wherein the product is selected from vitamin E, plastoquinone, a cyanogenic glycoside, a benzylisoquinoline alkaloid, rosmarinic acid, betalains, suberin, mescaline, morphine, salidroside, a phenylpropanoid compound, dhurrin, a tocochromanol, ubiquinone, lignin, a catecholamine, melanin, an isoquinoline alkaloid, hydroxycinnamic acid amide (HCAA), an amaryllidaceae alkaloid, hordenine, hydroxycinnamate, hydroxylstyrene, or tyrosine.

26. The method of any one of claims 21-25, further comprising purifying the product from the cell.
